(19) United States
(12) Patent Application Publication
(10) Pub. No.: US 2021/0198259 A1
(43) Pub. Date: Jul. 1, 2021

(54) HETEROCYCLIC DERIVATIVES AND USE THEREOF

(71) Applicant: C&C RESEARCH LABORATORIES, Gyeonggi-do (KR)

(72) Inventors: Kwang Seok KO, Gyeonggi-do (KR); Soon Ok LEE, Gyeonggi-do (KR); Young Jin KWON, Gyeonggi-do (KR); Jee Hun YUN, Gyeonggi-do (KR); Nam Chul CHO, Gyeonggi-do (KR); Ji Eun LEE, Gyeonggi-do (KR); Jung Sook KIM, Gyeonggi-do (KR)

(21) Appl. No.: 17/057,753

(22) PCT Filed: May 31, 2019
(86) PCT No.: PCT/KR2019/006553

§ 371 Date: Nov. 23, 2020

(30) Foreign Application Priority Data
May 31, 2018 (KR) 10-2018-0062254

(51) Int. Cl. C07D 471/14; A61P 17/04; C07D 519/00; C07D 487/04; C07D 513/14

(52) U.S. Cl. CPC C07D 471/14; A61P 17/04; C07D 513/14; C07D 487/04; C07D 519/00

(57) ABSTRACT

The present invention relates to novel heterocyclic compounds useful in preparing drugs for the treatment of diseases associated with various functions of the histamine 4 receptor. Specifically, these drugs are useful in the prevention or treatment of inflammatory disorder, allergy, pain, nasal polyps, rhinitis, chronic sinusitis, nasal congestion, nasal itch, asthma, chronic obstructive pulmonary disease, rheumatoid arthritis, atopic dermatitis, psoriasis, eczema, pruritus, itch skin, urticaria, idiopathic chronic urticaria, scleroderma, conjunctivitis, keratoconjunctivitis, ocular inflammation, dry eye, age-related macular degeneration, cardiac dysfunction, arrhythmia, atherosclerosis, multiple sclerosis, inflammatory bowel disease (colitis, Crohn's disease, ulcerative colitis), inflammatory pain, neuropathic pain, osteoarthritic pain, autoimmune thyroid disease, immune-mediated (also known as type I) diabetes, lupus, post-operative adhesions, vestibular disorders and cancer.

### TECHNICAL FIELD

The present invention relates to novel heterocyclic compounds useful in preparing drugs for the treatment of diseases associated with various functions of the histamine 4 receptor. Specifically, these drugs are useful in the prevention or treatment of inflammatory disorder, allergy, pain, nasal polyps, rhinitis, chronic sinusitis, nasal congestion, nasal itch, asthma, chronic obstructive pulmonary disease, rheumatoid arthritis, atopic dermatitis, psoriasis, eczema, pruritus, itch skin, urticaria, idiopathic chronic urticaria, scleroderma, conjunctivitis, keratoconjunctivitis, ocular inflammation, dry eye, age-related macular degeneration, cardiac dysfunction, arrhythmia, atherosclerosis, multiple sclerosis, inflammatory bowel disease (colitis, Crohn's disease, ulcerative colitis), inflammatory pain, neuropathic pain, osteoarthritic pain, autoimmune thyroid disease, immune-mediated (also known as type I) diabetes, lupus, post-operative adhesions, vestibular disorders and cancer.

### BACKGROUND ART

Histamine, which is a biogenic amine, plays a central role in the immune and inflammatory response and is also a neurotransmitter. For example, histamine controls various functions of antigen-presenting cells (dendritic cells and macrophages), T cells, B cells, epithelial and endothelial cells, and proliferation of T cells or cytokine secretion in dendritic cells and mast cells (W. Baumer et al., J. Dtsch. Dermatol. Ges. 2010, 8, 495-504). There are 4 histamine receptors (histamine 1 receptor, histamine 2 receptor, histamine 3 receptor and histamine 4 receptor) (M. E. Parsons et al., Br. J. Pharmacol. 2006, 147, S127-S135). An acute allergic reaction is controlled by the histamine 1 receptor which is distributed ubiquitously in the body (A. S. F. Ash et al., Br. J. Pharmacol. Chemother. 1966, 27, 427-439), and a gastric acid secretion is controlled by the histamine 2 receptor which also are distributed ubiquitously in the body like histamine 1 receptor (J. W. Black et al., Nature 1972, 236, 385-390). It is well known that the neurotransmitter secretion in the central nervous system is controlled by the histamine 3 receptor which is expressed in neurons (J. M. Arrang et al., Nature 1983, 302, 832-837). The histamine 4 receptor further explains physiological functions of many signaling processes which are not explained only by the histamine 1 receptor, histamine 2 receptor and histamine 3 receptor. The histamine 4 receptor was reported for the first time in 1994 and its cloning was performed only since 2000. The histamine 4 receptor, which is a G-protein coupled receptor, consists of 390 amino acids and is activated by binding with Gi/o protein to increase calcium concentration or suppress cyclic adenosine monophosphate (cAMP) (M. Shahid et al., The Open Immunology Journal, 2009, 2, 9-41). The histamine 4 receptor is mainly expressed in bone marrow or eosinophils, basophils, T cells, mast cells, monocytes and dendritic cells, and is also observed in the spleen, thymus, lung, heart and intestines (R. L. Thurmond et al., Nat. Rev. Drug Discov. 2008, 7, 41-53; T. Nakamura et al., Biochem. Biophys. Res. Commun. 2000, 279, 615-620). The histamine 4 receptor not only plays a central role in the immune response but also has effects on the activation and migration of various immunocytes, and the production of cytokines and chemokines (R. Gutzmer et al., J. Immunol. 2005, 174, 5224-5232; C. L. Hofstra et al., J. Pharmacol. Exp. Ther. 2003, 305, 1212-1221; D. Dijkstra et al., J. Allergy Clin. Immunol. 2007, 120, 300-307; M. O′Reilly et al., J. Recept. Signal Transduct. Res. 2002, 22, 431-448).

In various in vivo experiments, it is well known that the histamine 4 receptor plays an important role in inflammation and itch (P. J. Dunford et al., J. Allergy Clin. Immunol. 2007, 119, 176-183; R. L. Thurmond et al., J. Pharmacol. Exp. Ther. 2004, 309, 404-413). Specifically, as results of researches, it has been found in an allergic mouse asthma model that the histamine 4 antagonists alleviate lung inflammation by controlling Th2 (T helper type 2) reaction, and confirmed that histamine 4 antagonists effectively suppress histamine-induced itch. Such a dual effect against allergic inflammation and itch is a basis for the fact that the histamine 4 receptor may be a good target for treating allergic skin diseases such as atopic dermatitis (J. M. Cowden et al., J. Invest. Dermatol. 2010, 130, 1023-1033).

In such immunocytes, antagonism against the various functions of the histamine 4 receptor is a key focus of study of inflammatory diseases, pruritus, pain, allergic rhinitis, asthma, rheumatoid arthritis, atopic dermatitis, idiopathic chronic urticaria, inflammatory pain, neuropathic pain and osteoarthritic pain. Recently, the possibility as a therapeutic agent has been reported from the research results in which choroidal neo-vascularization in age-related macular degeneration was inhibited by a histamine 4 receptor antagonist (H. Kaneko et al., Br. J. Pharmacol. 2014, 171, 3754-3763).

In addition, a recent study related to the anticancer efficacy against the monotherapy or the combination therapy of the agonist for the histamine 4 receptor has been announced, and thereby its development as an anti-cancer drug is expected (W. K. Cal et al., Eur. J. Cancer 2014, 50, 1195-1206; N. A. Massari et al., Oncotarget 2017, 8, 26471-26491; A. M. B. Abiuso et al., Eur. J. Cancer 2018, 91, 125-135).

### DISCLOSURE OF INVENTION

### Technical Problem

Accordingly, the purpose of the present invention is the provision of a novel heterocyclic compounds regulating histamine 4 receptor.

Another purpose of the present invention is the provision of a pharmaceutical composition for the prevention or treatment of diseases associated with activation or inhibition of histamine 4 receptor.

### Solution to Problem

According to the present invention, there is provided a heterocyclic compound of the following Formula 1, or a pharmaceutically acceptable salt or isomer thereof:

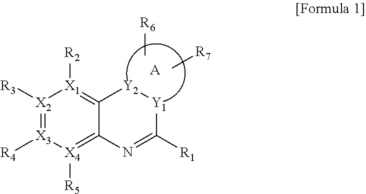

wherein
each of X₁, X₂, X₃ and X₄ is independently C or N;

R₁ is a saturated or unsaturated 3-12-membered mono- or poly-heterocyclyl containing 1-3 heteroatoms (preferably the heteroatoms selected from N, O and S), wherein R₁ is unsubstituted or substituted with 1-3 substituents selected from —C₁-C₆ alkyl and -amino-C₁-C₆ alkyl;

R₂, R₃, R₄ and R₅ may be the same or different; and each of them is independently selected from —H, —C₁-C₆ alkyl, —C₁-C₆haloalkyl, —C₁-C₆perhaloalkyl, -amino-C₁-C₆ alkyl, —C₃-C₈cycloalkyl, -halogen (—F, —Cl, —Br, —I), —CN, —C₁-C₆alkoxy, —C₁-C₆ haloalkoxy, —C₁-C₆perhaloalkoxy, —C₂-C₇ alkenyl, —C₂-C₈ alkynyl, -amino, -aceto, -amido, -sulfonamide, -sulfonyl, -aminosulfonyl-C₁-C₆alkyl, —C₁-C₆ alkylcarboxyl, -carboxyl (—COOH), —C₁-C₆acyl, —OH, -nitro (—NO₂), —C₆-C₁₀ aryl, -heterocyclyl, and —O—C₁-C₆alkyl-heterocyclyl, wherein the heterocyclyl is a saturated or unsaturated 3-6-membered heterocyclyl containing 1-3 heteroatoms (preferably heteroatoms selected from N, O and S);

provided that when X₁ is N, R₂ does not exist; when X₂ is N, R₃ does not exist; when X₃ is N, R₄ does not exist; and when X₄ is N, R₅ does not exist; and when all of X₁, X₂, X₃ and X₄ are C, R₃ is not hydrogen or fluorine (F);

each of Y₁ and Y₂ is independently C or N;

A ring is a saturated or unsaturated 5- or 6-membered heterocycle containing at least 2 heteroatoms (preferably the heteroatoms selected from N, O and S); and

each of R₆ and R₇ is independently oxo (═O) or ═NH, and one of R₆ and R₇ may not exist;

wherein each of the alkyl, cycloalkyl, heterocyclyl, alkoxy, alkenyl, alkynyl, acyl and aryl groups may be independently unsubstituted or substituted with one or more substituents (for example, 1-3 substituents) selected from the group consisting of —C₁-C₄ alkyl, -halogen (—F, —Cl, —Br, —I), —CN, —C₁-C₄ alkoxy, -amino, -amido, -carboxyl (—COOH), —C₁-C₆ acyl, —OH, -nitro (—NO₂), heterocyclyl and phenyl, wherein the heterocyclyl is a saturated or unsaturated 3-6-membered heterocyclyl containing 1-3 heteroatoms (preferably, the heteroatoms selected from N, O and S).

According to one embodiment of the present invention, in Formula 1,

each of X₁, X₂, X₃ and X₄ is independently C or N;

R₁ is a saturated or unsaturated 3-10-membered mono- or poly-heterocyclyl containing 1-3 heteroatoms selected from N, O and S, wherein the heterocyclyl is unsubstituted or substituted with 1 or 2 substituents selected from —C₁-C₆ alkyl and -amino-C₁-C₆ alkyl;

R₂, R₃, R₄ and R₅ may be the same or different; and each of them is independently selected from —H, —C₁-C₆ alkyl, —C₁-C₆haloalkyl, —C₁-C₆perhaloalkyl, -amino-C₁-C₆ alkyl, —C₃-C₈ cycloalkyl, -halogen, —CN, —C₁-C₆ alkoxy, —C₁-C₆haloalkoxy, —C₁-C₆ perhaloalkoxy, -amino, -aceto, -sulfonamino, -sulfonyl, -aminosulfonyl-C₁-C₆ alkyl, —C₁-C₆ alkylcarboxyl, -carboxyl, —OH, -nitro, —C₆-C₁₀ aryl, -heterocyclyl, and —O—C₁-C₆ alkyl-heterocyclyl, wherein the heterocyclyl is a saturated or unsaturated 3-6-membered heterocyclyl containing 1-3 heteroatoms selected from N, O and S;

each of Y₁ and Y₂ is independently C or N;

A ring is a saturated or unsaturated 5- or 6-membered heterocycle containing 2-4 heteroatoms selected from N, O and S; and

each of R₆ and R₇ is independently oxo or ═NH, and one of R₆ and R₇ may not exist.

According to another embodiment of the present invention, in Formula 1, X₁ and X₂ are C, and each of X₃ and X₄ is independently C or N.

According to still another embodiment of the present invention, in Formula 1, each of X₁ and X₂ is independently C or N, and X₃ and X₄ are C.

According to still another embodiment of the present invention, in Formula 1, R₁ is a saturated or unsaturated 3-10-membered mono- or poly-heterocyclyl containing 1-3 heteroatoms selected from N and O, wherein the heterocyclyl is unsubstituted or substituted with 1 or 2 substituents selected from —C₁-C₄ alkyl and -amino-C₁-C₄ alkyl.

According to still another embodiment of the present invention, in Formula 1, R₂, R₃, R₄ and R₅ may be the same or different; and each of them is independently selected from —H, —C₁-C₆ alkyl, —C₁-C₆haloalkyl, —C₁-C₆perhaloalkyl, -amino-C₁-C₆alkyl, -halogen, —CN, —C₁-C₆alkoxy, —C₁-C₆haloalkoxy, —C₁-C₆perhaloalkoxy, -amino, -aceto, -sulfonamino, -sulfonyl, -aminosulfonyl-C₁-C₆alkyl, —C₁-C₆ alkylcarboxyl, -carboxyl, —OH, -nitro, and -heterocyclyl, wherein the heterocyclyl is a saturated or unsaturated 5 or 6-membered heterocyclyl containing 1-3 heteroatoms selected from N, O and S.

According to still another embodiment of the present invention, in Formula 1, A ring is a saturated or unsaturated 5- or 6-membered heterocycle containing 2 or 3 heteroatoms selected from N and S.

According to still another embodiment of the present invention, in Formula 1, R₆ is oxo or ═NH, and R₇ does not exist.

According to still another embodiment of the present invention, in Formula 1, R₆ and R₇ are oxo.

Unless mentioned otherwise, alkyl substituent as described herein and alkyl residue in other substituents (for example, alkoxy) as described herein may be linear or branched. In addition, halogen includes fluorine (F), chlorine (Cl), bromine (Br) and iodine (I).

As representative examples of the compound of Formula 1 according to the present invention, the following compounds may be mentioned, but are not limited thereto:

8-bromo-4-(4-methylpiperazin-1-yl)imidazo[1,2-a]pyrido[2,3-e]pyrazin-2(1H)-one;

8-bromo-4-(3-(methylamino)azetidin-1-yl)imidazo[1,2-a]pyrido[2,3-e]pyrazin-2(1H)-one;

(R)-8-bromo-4-(3-(methylamino)pyrrolidin-1-yl)imidazo[1,2-a]pyrido[2,3-e]pyrazin-2(1H)-one;

(S)-8-bromo-4-(3-(methylamino)pyrrolidin-1-yl)imidazo[1,2-a]pyrido[2,3-e]pyrazin-2(1H)-one;

8-bromo-4-(hexahydropyrrolo[1,2-a]pyrazin-2(1H)-yl)imidazo[1,2-a]pyrido[2,3-e]pyrazin-2(1H)-one;

8-bromo-4-(4-methylpiperazin-1-yl)pyrido[2,3-e][1,2,4]triazolo[4,3-a]pyrazin-1(2H)-one;

8-bromo-4-(4-methylpiperazin-1-yl)pyrido[2,3-e][1,2,4]triazolo[1,5-a]pyrazin-2(1H)-one;

8-bromo-7-chloro-4-(4-methylpiperazin-1-yl)imidazo[1,2-a]pyrido[2,3-e]pyrazin-2(1H)-one;

8-bromo-7-fluoro-4-(4-methylpiperazin-1-yl)imidazo[1,2-a]pyrido[2,3-e]pyrazin-2(1H)-one;

4-(4-methylpiperazin-1-yl)-8-nitroimidazo[1,2-a]quinoxalin-2(1H)-one;

8-amino-4-(4-methylpiperazin-1-yl)imidazo[1,2-a]quinoxalin-2(1H)-one;

8-bromo-4-(4-methylpiperazin-1-yl)imidazo[1,2-a]quinoxalin-2(1H)-one;

8-bromo-4-(3-(methylamino)azetidin-1-yl)imidazo[1,2-a]quinoxalin-2(1H)-one;

9-bromo-5-(4-methylpiperazin-1-yl)-1H-[1,2,4]triazino[4,3-a]quinoxalin-2(3H)-one;

8,9-dibromo-4-(4-methylpiperazin-1-yl)imidazo[1,2-a]quinoxalin-2(1H)-one;

N-(4-(3-(methylamino)azetidin-1-yl)-2-oxo-1,2-dihydroimidazo[1,2-a]quinoxalin-8-yl)methanesulfonamide;

8-chloro-4-(4-methylpiperazin-1-yl)imidazo[1,2-a]pyrido[2,3-e]pyrazin-2(1H)-one;

8-amino-4-(3-(methylamino)azetidin-1-yl)imidazo[1,2-a]quinoxalin-2(1H)-one;

8-chloro-4-(3-(methylamino)azetidin-1-yl)imidazo[1,2-a]quinoxalin-2(1H)-one;

8-chloro-4-(3-(methylamino)azetidin-1-yl)imidazo[1,2-a]pyrido[2,3-e]pyrazin-2(1H)-one;

8-bromo-4-(3-(methylamino)azetidin-1-yl)imidazo[1,2-a]quinoxalin-2(1H)-one hydrochloride;

3-chloro-6-(4-methylpiperazin-1-yl)imidazo[1,2-a]pyrido[4,3-e]pyrazin-8(9H)-one;

8-chloro-4-(3-(methylamino)azetidin-1-yl)imidazo[1,2-a]pyrido[3,4-e]pyrazin-2(1H)-one hydrochloride;

8-chloro-4-(3-(methylamino)azetidin-1-yl)imidazo[1,2-a]pyrido[3,4-e]pyrazin-2(1H)-one;

8-bromo-4-(4-methylpiperazin-1-yl)imidazo[1,2-a]pyrido[3,4-e]pyrazin-2(1H)-one;

8-bromo-4-(3-(methylamino)azetidin-1-yl)imidazo[1,2-a]pyrido[3,4-e]pyrazin-2(1H)-one;

8-bromo-4-(4-methylpiperazin-1-yl)-1H-pyrido[2,3-e][1,2,4]thiadiazolo[4,3-a]pyrazine 2,2-dioxide;

8-chloro-4-(4-methylpiperazin-1-yl)imidazo[1,2-a]pyrido[3,4-e]pyrazin-2(1H)-one hydrochloride;

2-chloro-6-(4-methylpiperazin-1-yl)imidazo[1,2-a]pyrido[3,2-e]pyrazin-8(9H)-one;

2-bromo-6-(4-methylpiperazin-1-yl)imidazo[1,2-a]pyrido[3,2-e]pyrazin-8(9H)-one;

2-chloro-6-(3-(methylamino)azetidin-1-yl)imidazo[1,2-a]pyrido[3,2-e]pyrazin-8(9H)-one; and

2-bromo-6-(3-(methylamino)azetidin-1-yl)imidazo[1,2-a]pyrido[3,2-e]pyrazin-8(9H)-one.

The above-listed names of the compounds are described in accordance with the nomenclature method provided by ChemDraw Professional (Version 15.0.0.106) of PerkinElmer.

Because the compound of Formula 1 according to the present invention can have an asymmetric carbon center and asymmetric axis or plane, they can exist as E- or Z-isomer, R- or S-isomer, racemic mixtures or diastereoisomer mixtures and each diastereoisomer, all of which are within the scope of the present invention.

In case of the compound of Formula 1 according to the present invention being a racemate, the racemate may be separated into its respective isomers by using a conventional separation method, for example, a chiral column chromatography of normal-phase or reverse-phase, and employing the corresponding solvent, preferably a solvent mixture of hexane, ethyl acetate, dichloromethane and methanol in a normal-phase and a solvent mixture of water and acetonitrile in a reverse-phase.

The compound of Formula 1 according to the present invention may also form a pharmaceutically acceptable salt. Representative acids useful in preparing such a pharmaceutically acceptable salt (for example, acid addition salts) include, but not limited to, hydrochloric acid, sulfuric acid, nitric acid, phosphoric acid, hydrobromic acid, hydroiodic acid, formic acid, citric acid, acetic acid, trichloroacetic acid or trifluoroacetic acid, benzoic acid, fumaric acid, maleic acid, methane sulfonic acid, benzene sulfonic acid, p-toluene sulfonic acid, 2,2-dichloroacetic acid, acylated amino acids, adipic acid, alginic acid, ascorbic acid, L-aspartic acid, 4-acetamidobenzoic acid, (+)-camphoric acid, camphorsulfonic acid, (+)-(1S)-camphorsulfonic acid, capric acid, caproic acid, caprylic acid, cinnamic acid, cyclamic acid, dodecyl sulfuric acid, ethane-1,2-disulfonic acid, ethanesulfonic acid, 2-hydroxyethanesulfonic acid, galactaric acid, gentisic acid, glucoheptanoic acid, D-gluconic acid, D-glucuronic acid, L-glutamic acid, a-oxo-glutaric acid, glycolic acid, hippuric acid, (+)-L-lactic acid, (±)-DL-lactic acid, lactobionic acid, (−)-L-malic acid, malonic acid, (±)-DL-mandelic acid, methane sulfonic acid, naphthalene-2-sulfonic acid, naphthalene-1,5-disulfonic acid, 1-hydroxy-2-naphthoic acid, nicotinic acid, oleic acid, orotic acid, oxalic acid, palmitic acid, pamoic acid, L-pyroglutamic acid, salicylic acid, 4-amino-salicylic acid, sebacic acid, stearic acid, succinic acid, tannic acid, (+)-L-tartaric acid, thiocyanic acid, undecylenic acid and the like. In addition, other acid salts that are known and used in the art of amine derivatives may be included. They may be prepared by conventionally known processes.

The compound of Formula 1 as defined above according to the present invention may be prepared by, but not limited to, the methods described in the following Examples.

The compound of Formula 1 according to the present invention has an excellent activity for regulating human histamine 4 receptor (hH4R). Therefore, the present invention also provides a pharmaceutical composition comprising a therapeutically effective amount of a compound of Formula 1, or a pharmaceutically acceptable salt or isomer thereof as an active ingredient, and a pharmaceutically acceptable carrier.

The compound of Formula 1 according to the present invention is useful for preventing or treating inflammatory diseases, autoimmune diseases, allergic diseases, ocular diseases, skin diseases, respiratory diseases, pain diseases, cardiac diseases, and human histamine 4 receptor (hH4R)-related diseases.

Because the compound of Formula 1 according to the present invention shows a strong human histamine 4 receptor (hH4R) inhibitory activity, it is useful for preventing or treating inflammatory disorder, allergy, pain, nasal polyps, rhinitis, chronic sinusitis, nasal congestion, nasal itch, asthma, chronic obstructive pulmonary disease, rheumatoid arthritis, atopic dermatitis, psoriasis, eczema, pruritus, itchy skin, urticaria, idiopathic chronic urticaria, scleroderma, conjunctivitis, keratoconjunctivitis, ocular inflammation, dry eye, cardiac dysfunction, age-related macular degeneration, arrhythmia, atherosclerosis, multiple sclerosis, inflammatory bowel disease (colitis, Crohn's disease, ulcerative colitis), inflammatory pain, neuropathic pain, osteoarthritic pain, autoimmune thyroid disease, immune-mediated (also known as type I) diabetes, lupus, post-operative adhesions, vestibular disorders and cancer.

A pharmaceutical composition according to the present invention may be prepared by mixing a therapeutically effective amount of a compound of Formula 1, or a pharmaceutically acceptable salt or isomer thereof as an active ingredient, with a pharmaceutically acceptable carrier, binder, stabilizer and/or diluent. In addition, when the pharmaceutical composition according to the present invention is prepared in an injection liquid form, a pharmaceutically acceptable buffer, dissolution adjuvant and/or isotonic agent may be mixed with the compound of Formula 1, or a pharmaceutically acceptable salt or isomer thereof.

The pharmaceutical composition according to the prevent invention may be prepared in a delivery form of a pharmaceutical composition comprising one or more dosage units of pharmaceutical agent by using a preparation technique known or available to a skilled artisan, and a suitable pharmaceutical excipient. In a method of the present invention, the composition may be administered via suitable delivery route, for example, such as oral or parenteral, percutaneous, rectal, topical or ocular administration, or by inhalation. The pharmaceutical formulation may be in a form of tablet, capsule, sachet, sugar-coated pill, powder, granule, lozenge, powder for reconstitution, liquid preparation or suppository. For example, the composition may be formulated in a form for intravenous injection, spray, topical or oral administration.

In case of preparing a formulation in oral dosage form, any conventional pharmaceutical carriers may be used. For example, water, glycols, oils, alcohols and the like may be used as a carrier in case of oral liquid formulations such as suspensions, syrups, elixirs and solutions; and starches, sugars, kaolin, lubricants, binders, disintegrating agents and the like may be used as a carrier in case of solid formulations such as powders, pills, capsules and tablets. Because of the easiness of administration, tablets and capsules are the most convenient dose forms, and tablets and pills are preferably prepared as enteric coating formulations.

In case of parenteral formulations, sterilized water is used usually and other ingredient(s) such as a dissolution adjuvant may also be comprised. Injection formulations, for example, sterilized aqueous- or oil-based suspension for injection may be prepared according to known techniques by using appropriate dispersing agent, wetting agent or suspending agent. The solvents useful for this purpose include water, ringer solution and isotonic NaCl solution, and sterilized, immobilized oils are also used as a solvent or a suspending medium conventionally. Any non-irritant immobilized oils including mono- and di-glycerides may be used for this purpose, and fatty acids such as an oleic acid may be used for an injection formulation.

In case of percutaneous formulations, a penetration-enhancing agent and/or a suitable wetting agent may be used as a carrier, optionally in combination with suitable non-irritant additive(s) to the skin. As such additives, those helpful in enhancing the administration through the skin and/or preparing the desired composition may be selected. The percutaneous formulation may be administered in various ways, for example, such as a transdermal patch, a spot-on treatment or an ointment.

The administration time and dosage of the pharmaceutical composition according to the present invention may be suitably determined according to the patient's disease, condition, age, body weight and administration form. In case of adults, the pharmaceutical composition may be administered in an amount of 0.1-2,000 mg, preferably 1-200 mg per day, in a single dose or multiple doses, but not limited thereto.

### Advantageous Effects of Invention

The heterocyclic compound of Formula 1, or a pharmaceutically acceptable salt or isomer thereof according to the present invention exhibits an excellent effect on activating or inhibiting histamine 4 receptor, and thus a pharmaceutical composition comprising the same is useful in the prevention or treatment of diseases associated with regulating histamine 4 receptor. In addition, because the heterocyclic compound of Formula 1, or a pharmaceutically acceptable salt or isomer thereof according to the present invention has relatively long half-life, the heterocyclic compound of Formula 1, or a pharmaceutically acceptable salt or isomer thereof according to the present invention can regulate the activity of histamine 4 receptor for a relatively long time and can more effectively prevent or treat diseases associated with regulating histamine 4 receptor.

### MODE FOR THE INVENTION

Hereinafter, the present invention is explained in more detail with the following examples and experiments. However, the following examples and experiments are only intended to facilitate understanding of the present invention, and the protection scope of the present invention is not limited thereto.

The abbreviations used in the following examples are defined as follows.

| Abbreviation | Full Name |
|---|---|
| Brine | Brine is water, saturated or nearly saturated with |
| | salt (usually sodium chloride) |
| Celite | Trade name of diatomaceous earth |
| CDCl₃ | Deuterated chloroform |
| CH₃CN | Acetonitrile |
| CDI | Carbonyldiimidazole |
| CsF | Cesium fluoride |
| CuBr₂ | Copper(II) bromide |
| conc | Concentrated |
| DBU | 1,8-diazabicyclo[5.4.0]undec-7-ene |
| DCE | 1,2-dichloroethane |
| DCM | Dichloromethane |
| DIPEA | N,N-Diisopropylethylamine |
| DMF | N,N-Dimethylformamide |
| DMSO | Dimethylsulfoxide |
| DMSO-d₆ | Fully deuterated dimethylsulfoxide |
| EtOH | Ethyl alcohol |
| Et₂O | Diethyl ether |
| EtOAc | Ethyl acetate |
| Fe | Iron |
| H₂O | Water |
| HCl | Hydrochloric acid |
| n-Hex | n-Hexane |
| IPA | iso-Propyl alcohol |
| K₂CO₃ | Potassium carbonate |
| KOCN | Potassium cyanate |
| MeOH | Methyl alcohol |
| MgSO₄ | Magnesium sulfate |
| Na₂SO₄ | Sodium sulfate |
| NaH | Sodium hydride |
| NaHCO₃ | Sodium bicarbonate |
| NaOH | Sodium hydroxide |
| NH₃ | Ammonia |
| NH₄Cl | Ammonium chloride |
| POBr₃ | Phosphoryl bromide |
| POCl₃ | Phosphoryl chloride |
| SOCl₂ | Thionyl chloride |
| TBAC | Tetrabutylammonium chloride |
| TEA | Triethylamine |
| TFA | Trifluoroacetic acid |
| THF | Tetrahydrofuran |

### Example 1: Synthesis of 8-bromo-4-(4-methylpiperazin-1-yl)imidazo[1,2-a]pyrido[2,3-e]pyrazin-2(1H)-one

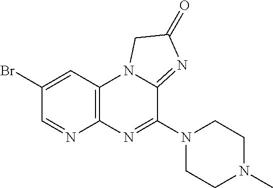 [SMILES: CN1CCN(C2=Nc3ncc(Br)cc3N3CC(=O)N=C23)CC1]

(a) Synthesis of 7-bromo-2-chloro-3-(4-methylpiperazin-1-yl)pyrido[2,3-b]pyrazine

7-Bromo-2,3-dichloropyrido[2,3-b]pyrazine (10.0 g, 35.9 mmol) and 1-methylpiperazine (3.78 mL, 34.1 mmol) were dissolved in DCM (359 mL), and TEA (15.0 mL, 108 mmol) was slowly added thereto at 0° C. The reaction mixture was stirred at room temperature for 15 hours, H₂O (250 mL) was added thereto, and extracted with DCM (250 mL). The organic layer was washed with brine, dried over anhydrous MgSO₄, filtered and distilled under reduced pressure. The residue was purified by column chromatography (n-Hex:EtOAc=2:1) on amine silica, and the fractions containing the product fractions were collected and evaporated to obtain the solid compound, 7-bromo-2-chloro-3-(4-methylpiperazin-1-yl)pyrido[2,3-b]pyrazine (8.50 g, 69%) in brown.

LC/MS ESI (+): 342 (M+1)

¹H NMR (400 MHz, CDCl₃) δ=8.94; (d, J=2.4 Hz, 1H), 8.33; (d, J=2.4 Hz, 1H), 3.81-3.79; (m, 4H), 2.65-2.62; (m, 4H), 2.38; (s, 3H)

(b) Synthesis of N-(7-bromo-3-(4-methylpiperazin-1-yl)pyrido[2,3-b]pyrazin-2-yl)-2-hydroxyacetamide

7-Bromo-2-chloro-3-(4-methylpiperazin-1-yl)pyrido[2,3-b]pyrazine (500 mg, 1.46 mmol) and glycolamide (131 mg, 1.75 mmol) were dissolved in DMF (14.6 mL), and anhydrous K₂CO₃ (303 mg, 2.19 mmol) was added thereto at room temperature. The reaction mixture was stirred at 70° C. for 1 hour and distilled under reduced pressure. The residue was purified by column chromatography (H₂O containing 0.1% formic acid:CH₃CN=70:30) on reverse-phase silica and column chromatography (DCM:MeOH=20:1) on amine silica, and the fractions containing the product were collected and evaporated to obtain the solid compound, N-(7-bromo-3-(4-methylpiperazin-1-yl)pyrido[2,3-b]pyrazin-2-yl)-2-hydroxyacetamide (351 mg, 63%) in yellow.

LC/MS ESI (+): 381 (M+1)

¹H NMR (400 MHz, DMSO-d₆) δ=8.69; (d, J=2.3 Hz, 1H), 8.19; (d, J=2.3 Hz, 1H), 7.59; (br s, 1H), 7.30; (br s, 1H), 4.94; (s, 2H), 3.88-3.86; (m, 4H), 2.50-2.47; (m, 4H), 2.23; (s, 3H)

(c) Synthesis of 8-bromo-4-(4-methylpiperazin-1-yl)imidazo[1,2-a]pyrido[2,3-e]pyrazin-2(1H)-one

N-(7-bromo-3-(4-methylpiperazin-1-yl)pyrido[2,3-b]pyrazin-2-yl)-2-hydroxyacetamide (351 mg, 0.921 mmol) was dissolved in DMF (18.4 mL), and methanesulfonyl chloride (2.15 mL, 27.6 mmol) and TEA (3.85 mL, 27.6 mmol) were added thereto at room temperature. The reaction mixture was stirred at 80° C. for 1 hour and distilled under reduced pressure. The residue was purified by column chromatography (H₂O containing 0.1% formic acid:CH₃CN=65:35) on reverse-phase silica and column chromatography (DCM:MeOH=20:1) on amine silica, and the fractions containing the product were collected and evaporated to obtain the solid compound, 8-bromo-4-(4-methylpiperazin-1-yl)imidazo[1,2-a]pyrido[2,3-e]pyrazin-2(1H)-one (207 mg, 62%) in brown.

LC/MS ESI (+): 363 (M+1)

¹H NMR (400 MHz, DMSO-d₆) δ=8.77; (d, J=2.4 Hz, 1H), 8.34; (d, J=2.4 Hz, 1H), 5.40; (s, 2H), 3.82-3.79; (m, 4H), 2.50-2.47; (m, 4H), 2.24; (s, 3H)

### Example 2: Synthesis of 8-bromo-4-(3-(methylamino)azetidin-1-yl)imidazo[1,2-a]pyrido[2,3-e]pyrazin-2(1H)-one

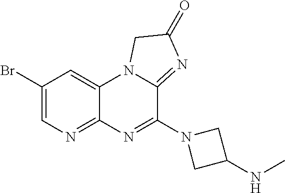 [SMILES: CNC1CN(C2=Nc3ncc(Br)cc3N3CC(=O)N=C23)C1]

(a) Synthesis of tert-butyl (1-(7-bromo-2-chloropyrido[2,3-b]pyrazin-3-yl)azetidin-3-yl)(methyl)carbamate

7-Bromo-2,3-dichloropyrido[2,3-b]pyrazine (10.0 g, 35.9 mmol) and tert-butyl azetidin-3-yl(methyl)carbamate (6.68 g, 35.9 mmol) were dissolved in toluene (179 mL), and TEA (9.99 mL, 71.7 mmol) was slowly added thereto at 0° C. The reaction mixture was stirred at 0° C. for 1 hour, H₂O (250 mL) was added thereto, and extracted with DCM (250 mL). The organic layer was washed with brine, dried over anhydrous MgSO₄, filtered and distilled under reduced pressure. The residue was purified by column chromatography (n-Hex:EtOAc=4:1) on silica, and the fractions containing the product were collected and evaporated to obtain the solid compound, tert-butyl (1-(7-bromo-2-chloropyrido[2,3-b]pyrazin-3-yl)azetidin-3-yl)(methyl)carbamate (7.00 g, 46%) in yellow.

LC/MS ESI (+): 428 (M+1)

¹H NMR (400 MHz, CDCl₃) δ=8.86; (d, J=2.4 Hz, 1H), 8.25; (d, J=2.4 Hz, 1H), 5.17-4.92; (m, 1H), 4.76; (br s, 2H), 4.57-4.53; (m, 2H), 2.98; (s, 3H), 1.48; (s, 9H)

(b) Synthesis of tert-butyl (1-(7-bromo-2-(2-hydroxyacetamido)pyrido[2,3-b]pyrazin-3-yl)azetidin-3-yl)(methyl)carbamate

Tert-butyl (1-(7-bromo-2-chloropyrido[2,3-b]pyrazin-3-yl)azetidin-3-yl)(methyl)carbamate (500 mg, 1.17 mmol) and glycolamide (105 mg, 1.40 mmol) were dissolved in DMF (5.83 mL), and anhydrous K₂CO₃ (242 mg, 1.75 mmol) was added thereto at room temperature. The reaction mixture was stirred at 70° C. for 1 hour and distilled under reduced pressure. The residue was purified by column chromatography (H₂O containing 0.1% formic acid:CH₃CN=60:40) on reverse-phase silica and column chromatography (DCM:MeOH=50:1) on amine silica, and the fractions containing the product were collected and evaporated to obtain the solid compound, tert-butyl (1-(7-bromo-2-(2-hydroxyacetamido)pyrido[2,3-b]pyrazin-3-yl)azetidin-3-yl)(methyl)carbamate (330 mg, 61%) in yellow.

LC/MS ESI (+): 467 (M+1)

¹H NMR (400 MHz, DMSO-d₆) δ=8.62; (d, J=2.3 Hz, 1H), 8.14; (d, J=2.3 Hz, 1H), 7.55; (s, 1H), 7.35; (s, 1H), 4.99-4.27; (m, 7H), 2.91; (s, 3H), 1.42; (s, 9H)

(c) Synthesis of tert-butyl (1-(8-bromo-2-oxo-1,2-dihydroimidazo[1,2-a]pyrido[2,3-e]pyrazin-4-yl)azetidin-3-yl)(methyl)carbamate

Tert-butyl (1-(7-bromo-2-(2-hydroxyacetamido)pyrido[2,3-b]pyrazin-3-yl)azetidin-3-yl)(methyl)carbamate (330 mg, 0.706 mmol) was dissolved in DMF (7.06 mL), and methanesulfonyl chloride (1.65 mL, 21.2 mmol) and TEA (2.95 mL, 21.2 mmol) were added thereto at room temperature. The reaction mixture was stirred at 80° C. for 1 hour and distilled under reduced pressure. The residue was purified by column chromatography (H₂O containing 0.1% formic acid:CH₃CN=65:35) on reverse-phase silica and column chromatography (DCM:MeOH=50:1) on amine silica, and the fractions containing the product were collected and evaporated to obtain the solid compound, tert-butyl (1-(8-bromo-2-oxo-1,2-dihydroimidazo[1,2-a]pyrido[2,3-e]pyrazin-4-yl)azetidin-3-yl)(methyl)carbamate (33.0 mg, 10%) in brown.

LC/MS ESI (+): 449 (M+1)

¹H NMR (400 MHz, CDCl₃) δ=8.73; (d, J=2.4 Hz, 1H), 8.15; (d, J=2.3 Hz, 1H), 5.14; (s, 3H), 4.65; (br s, 2H), 4.49; (br s, 2H), 2.97; (s, 3H), 1.48; (s, 9H)

(d) Synthesis of 8-bromo-4-(3-(methylamino)azetidin-1-yl)imidazo[1,2-a]pyrido[2,3-e]pyrazin-2(1H)-one

Tert-butyl (1-(8-bromo-2-oxo-1,2-dihydroimidazo[1,2-a]pyrido[2,3-e]pyrazin-4-yl)azetidin-3-yl)(methyl)carbamate (33.0 mg, 0.0730 mmol) was dissolved in DCM (0.294 mL), and TFA (0.169 mL) was added thereto. The reaction mixture was stirred at room temperature for 1 hour. DIPEA (0.300 mL) was added to the reaction mixture at 0° C., and the solvent was evaporated under reduced pressure. The residue was purified by column chromatography (DCM:MeOH =50:1) on amine silica, and the fractions containing the product were collected and evaporated to obtain the solid compound, 8-bromo-4-(3-(methylamino)azetidin-1-yl)imidazo[1,2-a]pyrido[2,3-e]pyrazin-2(1H)-one (18.0 mg, 70%) in yellow.

LC/MS ESI (+): 349 (M+1)

¹H NMR (400 MHz, DMSO-d₆) δ=8.60; (d, J=2.4 Hz, 1H), 8.16; (d, J=2.4 Hz, 1H), 5.28; (s, 2H), 4.70-3.77; (m, 4H), 3.57-3.51; (m, 1H), 2.20; (s, 3H)

### Example 3: Synthesis of (R)-8-bromo-4-(3-(methylamino)pyrrolidin-1-yl)imidazo[1,2-a]pyrido[2,3-e]pyrazin-2(1H)-one

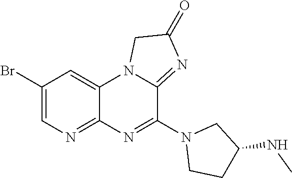 [SMILES: CN[C@@H]1CCN(C2=Nc3ncc(Br)cc3N3CC(=O)N=C23)C1]

(a) Synthesis of tert-butyl (R)-(1-(7-bromo-2-chloropyrido[2,3-b]pyrazin-3-yl)pyrrolidin-3-yl)(methyl)carbamate

7-Bromo-2,3-dichloropyrido[2,3-b]pyrazine (600 mg, 2.15 mmol) and tert-butyl (R)-methyl(pyrrolidin-3-yl)carbamate (392 mg, 1.96 mmol) were dissolved in DCM (19.6 mL), and TEA (0.818 mL, 5.87 mmol) was slowly added thereto at 0° C. The reaction mixture was stirred at 25° C. for 1 hour, H₂O (25.0 mL) was added thereto, and extracted with DCM (25.0 mL). The organic layer was washed with brine, dried over anhydrous MgSO₄, filtered and distilled under reduced pressure. The residue was purified by column chromatography (n-Hex:EtOAc=50:50) on amine silica, and the fractions containing the product were collected and evaporated to obtain the solid compound, tert-butyl (R)-(1-(7-bromo-2-chloropyrido[2,3-b]pyrazin-3-yl)pyrrolidin-3-yl)(methyl)carbamate (582 mg, 67%) in yellow.

LC/MS ESI (+): 442 (M+1)

¹H NMR (400 MHz, CDCl₃) δ=8.86; (d, J=2.4 Hz, 1H), 8.25; (d, J=2.4 Hz, 1H), 4.84; (br s, 1H), 4.15-4.06; (m, 2H), 4.00-3.86; (m, 2H), 2.86; (s, 3H), 2.21-2.10; (m, 2H), 1.49; (s, 9H)

(b) Synthesis of tert-butyl (R)-(1-(7-bromo-2-(2-hydroxyacetamido)pyrido[2,3-b]pyrazin-3-yl)pyrrolidin-3-yl)(methyl)carbamate

Tert-butyl (R)-(1-(7-bromo-2-chloropyrido[2,3-b]pyrazin-3-yl)pyrrolidin-3-yl)(methyl)carbamate (582 mg, 1.32 mmol) and glycolamide (118 mg, 1.58 mmol) were dissolved in DMF (6.57 mL), and anhydrous K₂ CO₃ (273 mg, 1.97 mmol) was added thereto at room temperature. The reaction mixture was stirred at 70° C. for 1 hour and distilled under reduced pressure. The residue was purified by column chromatography (H₂O containing 0.1% formic acid:CH₃CN=55:45) on reverse-phase silica and column chromatography (DCM:MeOH=50:1) on amine silica, and the fractions containing the product were collected and evaporated to obtain the solid compound, tert-butyl (R)-(1-(7-bromo-2-(2-hydroxyacetamido)pyrido[2,3-b]pyrazin-3-yl)pyrrolidin-3-yl)(methyl)carbamate (460 mg, 73%) in yellow.

LC/MS ESI (+): 481 (M+1)

¹H NMR (400 MHz, CDCl₃) δ=8.68; (d, J=2.4 Hz, 1H), 8.07; (d, J=2.4 Hz, 1H), 6.10; (br s, 1H), 5.59; (br s, 1H), 5.06-4.96; (m, 2H), 4.79; (br s, 1H), 4.18-4.13; (m, 2H), 3.91-3.79; (m, 2H), 2.85; (s, 3H), 2.22-2.04; (m, 2H), 1.48; (s, 9H)

(c) Synthesis of tert-butyl (R)-(1-(8-bromo-2-oxo-1,2-dihydroimidazo[1,2-a]pyrido[2,3-e]pyrazin-4-yl)pyrrolidin-3-yl)(methyl)carbamate

Tert-butyl (R)-(1-(7-bromo-2-(2-hydroxyacetamido)pyrido[2,3-b]pyrazin-3-yl)pyrrolidin-3-yl)(methyl)carbamate (460 mg, 0.956 mmol) was dissolved in DMF (9.56 mL), and methanesulfonyl chloride (0.371 mL, 4.78 mmol) and TEA (0.666 mL, 4.78 mmol) were added thereto at room temperature. The reaction mixture was stirred at 80° C. for 1 hour and distilled under reduced pressure. The residue was purified by column chromatography (H₂O containing 0.1% formic acid:CH₃CN=40:60) on reverse-phase silica and column chromatography (DCM:MeOH=90:10) on amine silica, and the fractions containing the product were collected and evaporated to obtain the solid compound, tert-butyl (R)-(1-(8-bromo-2-oxo-1,2-dihydroimidazo[1,2-a]pyrido[2,3-e]pyrazin-4-yl)pyrrolidin-3-yl)(methyl)carbamate (125 mg, 28%) in brown.

LC/MS ESI (+): 463 (M+1)

¹H NMR (400 MHz, CDCl₃) δ=8.72; (d, J=2.4 Hz, 1H), 8.14; (d, J=2.4 Hz, 1H), 5.15; (s, 2H), 4.85; (br s, 1H), 4.18-4.08; (m, 2H), 3.91-3.84; (m, 1H), 3.79; (dd, J=12.2, 8.1 Hz, 1H), 2.85; (s, 3H), 2.23-2.03; (m, 2H), 1.49; (s, 9H)

(d) Synthesis of (R)-8-bromo-4-(3-(methylamino)pyrrolidin-1-yl)imidazo[1,2-a]pyrido[2,3-e]pyrazin-2(1H)-one

Tert-butyl (R)-(1-(8-bromo-2-oxo-1,2-dihydroimidazo[1,2-a]pyrido[2,3-e]pyrazin-4-yl)pyrrolidin-3-yl)(methyl)carbamate (125 mg, 0.270 mmol) was dissolved in DCM (1.08 mL), and TFA (0.620 mL) was added thereto. The reaction mixture was stirred at room temperature for 1 hour. DIPEA (1.20 mL) was added to the reaction mixture at 0° C., and the solvent was evaporated under reduced pressure. The residue was purified by column chromatography (DCM:MeOH=100:1) on amine silica, and the fractions containing the product were collected and evaporated to obtain the solid compound, (R)-8-bromo-4-(3-(methylamino)pyrrolidin-1-yl)imidazo[1,2-a]pyrido[2,3-e]pyrazin-2(1H)-one (81.0 mg, 83%) in yellow.

LC/MS ESI (+): 363 (M+1)

¹H NMR (400 MHz, DMSO-d₆) δ=8.65; (d, J=2.4 Hz, 1H), 8.20; (d, J=2.4 Hz, 1H), 5.36; (s, 2H), 3.89; (br s, 2H), 3.65; (br s, 1H), 3.26-3.22; (m, 1H), 2.31; (s, 3H), 2.08-2.00; (m, 2H), 1.87-1.80; (m, 1H)

### Example 4: Synthesis of (S)-8-bromo-4-(3-(methylamino)pyrrolidin-1-yl)imidazo[1.2-a]pyrido[2.3-e]pyrazin-2(1H)-one

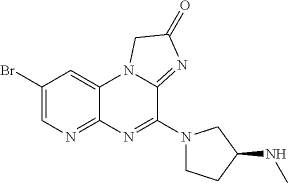 [SMILES: CN[C@H]1CCN(C2=Nc3ncc(Br)cc3N3CC(=O)N=C23)C1]

(a) Synthesis of tert-butyl (S)-(1-(7-bromo-2-chloropyrido[2,3-b]-pyrazin-3-yl)pyrrolidin-3-yl)(methyl)carbamate

7-Bromo-2,3-dichloropyrido[2,3-b]pyrazine (300 mg, 1.08 mmol) and tert-butyl (S)-methyl(pyrrolidin-3-yl)carbamate (196 mg, 0.978 mmol) were dissolved in DCM (9.78 mL), and TEA (0.409 mL, 2.93 mmol) was slowly added thereto at 0° C. The reaction mixture was stirred at 25° C. for 1 hour, H₂O (10.0 mL) was added thereto, and extracted with DCM (10.0 mL). The organic layer was washed with brine, dried over anhydrous MgSO₄, filtered and distilled under reduced pressure. The residue was purified by column chromatography (n-Hex:EtOAc=2:1) on amine silica, and the fractions containing the product were collected and evaporated to obtain the solid compound, tert-butyl (S)-(1-(7-bromo-2-chloropyrido[2,3-b]pyrazin-3-yl)pyrrolidin-3-yl)(methyl)carbamate (303 mg, 70%) in yellow.

LC/MS ESI (+): 442 (M+1)

¹H NMR (400 MHz, CDCl₃) δ=8.86; (d, J=2.4 Hz, 1H), 8.25; (d, J=2.4 Hz, 1H), 4.91-4.75; (m, 1H), 4.15-4.05; (m, 2H), 4.00-3.93; (m, 1H), 3.88; (dd, J=11.6, 8.3 Hz, 1H), 2.86; (s, 3H), 2.23-2.09; (m, 2H), 1.49; (s, 9H)

(b) Synthesis of tert-butyl (S)-(1-(7-bromo-2-(2-hydroxyacetamido)pyrido[2,3-b]pyrazin-3-yl)pyrrolidin-3-yl)(methyl)carbamate

Tert-butyl (S)-(1-(7-bromo-2-chloropyrido[2,3-b]pyrazin-3-yl)pyrrolidin-3-yl)(methyl)carbamate (303 mg, 0.684 mmol) and glycolamide (61.6 mg, 0.821 mmol) were dissolved in DMF (3.42 mL), and anhydrous K₂ CO₃ (142 mg, 1.03 mmol) was added thereto at room temperature. The reaction mixture was stirred at 70° C. for 1 hour and distilled under reduced pressure. The residue was purified by column chromatography (DCM:MeOH=60:1) on amine silica, and the fractions containing the product were collected and evaporated to obtain the solid compound, tert-butyl (S)-(1-(7-bromo-2-(2-hydroxyacetamido)pyrido[2,3-b]pyrazin-3-yl)pyrrolidin-3-yl)(methyl)carbamate (240 mg, 73%) in yellow.

LC/MS ESI (+): 481 (M+1)

¹H NMR (400 MHz, CDCl₃) δ=8.68; (d, J=2.3 Hz, 1H), 8.07; (d, J=2.3 Hz, 1H), 6.05; (br s, 1H), 5.55; (br s, 1H), 5.05-4.97; (m, 2H), 4.79; (br s, 1H), 4.19-4.14; (m, 2H), 3.92-3.80; (m, 2H), 2.85; (s, 3H), 2.22-2.07; (m, 2H), 1.48 (s, 9H)

(c) Synthesis of tert-butyl (S)-(1-(8-bromo-2-oxo-1,2-dihydroimidazo[1,2-a]pyrido[2,3-e]pyrazin-4-yl)pyrrolidin-3-yl)(methyl)carbamate

Tert-butyl (S)-(1-(7-bromo-2-(2-hydroxyacetamido)pyrido[2,3-b]pyrazin-3-yl)pyrrolidin-3-yl)(methyl)carbamate (240 mg, 0.499 mmol) was dissolved in DMF (4.99 mL), and methanesulfonyl chloride (0.387 mL, 4.99 mmol) and TEA (0.695 mL, 4.99 mmol) were added thereto at room temperature. The reaction mixture was stirred at 80° C. for 1 hour and distilled under reduced pressure. The residue was purified by column chromatography (H₂O containing 0.1% formic acid:CH₃CN=40:60) on reverse-phase silica and column chromatography (DCM:MeOH=19:1) on silica, and the fractions containing the product were collected and evaporated to obtain the solid compound, tert-butyl (S)-(1-(8-bromo-2-oxo-1,2-dihydroimidazo[1,2-a]pyrido[2,3-e]pyrazin-4-yl)pyrrolidin-3-yl)(methyl)carbamate (117 mg, 51%) in brown.

LC/MS ESI (+): 463 (M+1)

¹H NMR (400 MHz, CDCl₃) δ=8.72; (d, J=2.4 Hz, 1H), 8.14 (d, J=2.4 Hz, 1H), 5.15; (s, 2H), 4.84; (br s, 1H), 4.17-4.08; (m, 2H), 3.92-3.84; (m, 1H), 3.81-3.76; (m, 1H), 2.85; (s, 3H), 2.21-2.08; (m, 2H), 1.49; (s, 9H)

(d) Synthesis of (S)-8-bromo-4-(3-(methylamino)pyrrolidin-1-yl)imidazo[1,2-a]pyrido[2,3-e]pyrazin-2(1H)-one

Tert-butyl (S)-(1-(8-bromo-2-oxo-1,2-dihydroimidazo[1,2-a]pyrido[2,3-e]pyrazin-4-yl)pyrrolidin-3-yl)(methyl)carbamate (115 mg, 0.248 mmol) was dissolved in DCM (0.993 mL), and TFA (0.570 mL) was added thereto. The reaction mixture was stirred at room temperature for 1 hour. DIPEA (1.20 mL) was added to the reaction mixture at 0° C., and the solvent was evaporated under reduced pressure. The residue was purified by column chromatography (DCM:MeOH=100:1) on amine silica, and the fractions containing the product were collected and evaporated to obtain the solid compound, (S)-8-bromo-4-(3-(methylamino)pyrrolidin-1-yl)imidaz[1,2-a]pyrido[2,3-e]pyrazin-2(1H)-one (75.0 mg, 83%) in yellow.

LC/MS ESI (+): 363 (M+1)

¹H NMR (400 MHz, DMSO-d₆) δ=8.65; (d, J=2.4 Hz, 1H), 8.20; (d, J=2.4 Hz, 1H), 5.36; (s, 2H), 3.87; (br s, 2H), 3.64; (br s, 1H), 3.26-3.21; (m, 1H), 2.31; (s, 3H), 2.08-2.00; (m, 2H), 1.87-1.81; (m, 1H)

### Example 5: Synthesis of 8-bromo-4-(hexahydropyrrolo[1,2-a]pyrazin-2(1H)-yl)imidazo[1,2-a]pyrido[2,3-e]pyrazin-2(1H)-one

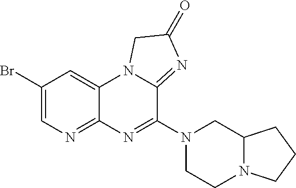 [SMILES: O=C1CN2C(=N1)C(N1CCN3CCCC3C1)=Nc1ncc(Br)cc12]

(a) Synthesis of 7-bromo-2-chloro-3-(hexahydropyrrolo[1,2-a]pyrazin-2(1H)-yl)pyrido[2,3-b]pyrazine

7-Bromo-2,3-dichloropyrido[2,3-b]pyrazine (300 mg, 1.08 mmol) and octahydropyrrolo[1,2-a]pyrazine (123 mg, 0.978 mmol) were dissolved in DCM (9.78 mL), and TEA (0.409 mL, 2.93 mmol) was slowly added thereto at 0° C. The reaction mixture was stirred at 25° C. for 1 hour, H₂O (10.0 mL) was added thereto, and extracted with DCM (10.0 mL). The organic layer was washed with brine, dried over anhydrous MgSO₄, filtered and distilled under reduced pressure. The residue was purified by column chromatography (n-Hex:EtOAc=2:1) on amine silica, and the fractions containing the product were collected and evaporated to obtain the solid compound, 7-bromo-2-chloro-3-(hexahydropyrrolo[1,2-a]pyrazin-2(1H)-yl)pyrido[2,3-b]pyrazine (165 mg, 46%) in yellow.

LC/MS ESI (+): 368 (M+1)

¹H NMR (400 MHz, CDCl₃) δ=8.93 (d, J=2.4 Hz, 1H), 8.33; (d, J=2.4 Hz, 1H), 4.51-4.40; (m, 2H), 3.31-3.24; (m, 1H), 3.19-3.13; (m, 2H), 2.96; (dd, J=12.5, 10.5 Hz, 1H), 2.49; (td, J=11.4, 3.1 Hz, 1H), 2.27-2.20; (m, 2H), 1.96-1.86; (m, 2H), 1.83-1.72; (m, 1H), 1.56-1.46; (m, 1H)

(b) Synthesis of N-(7-bromo-3-(hexahydropyrrolo[1,2-a]pyrazin-2(1H)-yl)pyrido[2,3-b]pyrazin-2-yl)-2-hydroxyacetamide

7-Bromo-2-chloro-3-(hexahydropyrrolo[1,2-a]pyrazin-2(1H)-yl)pyrido[2,3-b]pyrazine (163 mg, 0.442 mmol) and glycolamide (39.8 mg, 0.531 mmol) were dissolved in DMF (4.42 mL), and anhydrous K₂CO₃ (92.0 mg, 0.663 mmol) was added thereto at room temperature. The reaction mixture was stirred at 70° C. for 1 hour and distilled under reduced pressure. The residue was purified by column chromatography (H₂O containing 0.1% formic acid:CH₃CN=75:25) on reverse-phase silica and column chromatography (DCM:MeOH=30:1) on amine silica, and the fractions containing the product were collected and evaporated to obtain the solid compound, N-(7-bromo-3-(hexahydropyrrolo[1,2-a]pyrazin-2(1H)-yl)pyrido[2,3-b]pyrazin-2-yl)-2-hydroxyacetamide (75.0 mg, 42%) in yellow.

LC/MS ESI (+): 407 (M+1)

¹H NMR (400 MHz, CDCl₃) δ=8.75; (d, J=2.3 Hz, 1H), 8.15; (d, J=2.4 Hz, 1H), 6.09-5.99; (m, 1H), 5.58-5.47; (m, 1H), 5.05; (d, J=1.7 Hz, 2H), 4.80-4.75; (m, 1H), 4.71-4.65; (m, 1H), 3.32-3.24; (m, 1H), 3.19-3.12; (m, 2H), 2.93; (dd, J=12.6, 10.5 Hz, 1H), 2.41; (td, J=11.5, 3.1 Hz, 1H), 2.24-2.13; (m, 2H), 1.95-1.84; (m, 2H), 1.82-1.72; (m, 1H), 1.52-1.47; (m, 1H)

(c) Synthesis of 8-bromo-4-(hexahydropyrrolo[1,2-a]pyrazin-2(1H)-yl)imidazo[1,2-a]pyrido[2,3-e]pyrazin-2(1H)-one

N-(7-bromo-3-(hexahydropyrrolo[1,2-a]pyrazin-2(1H)-yl)pyrido[2,3-b]pyrazin-2-yl)-2-hydroxyacetamide (74.0 mg, 0.182 mmol) was dissolved in DMF (3.63 mL), and methanesulfonyl chloride (0.282 mL, 3.63 mmol) and TEA (0.507 mL, 3.63 mmol) were added thereto at room temperature. The reaction mixture was stirred at 80° C. for 15 hours and distilled under reduced pressure. The residue was purified by column chromatography (H₂O containing 0.1% formic acid:CH₃CN=70:30) on reverse-phase silica and column chromatography (DCM:MeOH=9:1) on silica, and the fractions containing the product were collected and evaporated to obtain the solid compound, 8-bromo-4-(hexahydropyrrolo[1,2-a]pyrazin-2(1H)-yl)imidazo[1,2-a]pyrido[2,3-e]pyrazin-2(1H)-one (14.0 mg, 20%) in yellow.

LC/MS ESI (+): 389 (M+1)

¹H NMR (400 MHz, DMSO-d₆) δ=8.77 (d, J=2.3 Hz, 1H), 8.34; (d, J=2.4 Hz, 1H), 5.40; (s, 2H), 4.65; (d, J=12.6 Hz, 1H), 4.55; (d, J=12.7; Hz, 1H), 3.18-3.01; (m, 3H), 2.83; (t, J=11.4 Hz, 1H), 2.30-2.24; (m, 1H), 2.13-2.05; (m, 2H), 1.87-1.81; (m, 1H), 1.78-1.67; (m, 2H), 1.45-1.35; (m, 1H)

### Example 6: Synthesis of 8-bromo-4-(4-methylpiperazin-1-yl)pyrido[2,3-e][1,2,4] triazolo[4,3-a]pyrazin-1(2H)-one

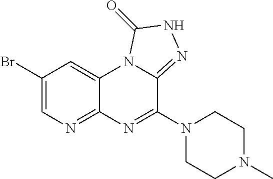 [SMILES: CN1CCN(c2nc3ncc(Br)cc3n3c(=O)[nH]nc23)CC1]

(a) Synthesis of 7-bromo-2-hydrazinyl-3-(4-methylpiperazin-1-yl)pyrido[2,3-b]pyrazine

7-Bromo-2-chloro-3-(4-methylpiperazin-1-yl)pyrido[2,3-b]pyrazine (250 mg, 0.730 mmol) obtained in step (a) of Example 1 and hydrazine monohydrate (0.142 mL, 2.92 mmol) were dissolved in EtOH (1.46 mL), stirred at room temperature for 30 minutes and distilled under reduced pressure. Et₂O was added to the residue, and the resulting mixture was filtered and dried under reduced pressure to obtain the solid compound, 7-bromo-2-hydrazinyl-3-(4-methylpiperazin-1-yl)pyrido[2,3-b]pyrazine (247 mg, 100%) in yellow.

LC/MS ESI (+): 338 (M+1)

¹H NMR (400 MHz, DMSO-d₆) δ=8.01-7.45; (m, 1H), 7.09; (br s, 4H), 3.96-3.74; (m, 4H), 2.43; (br s, 4H), 2.21; (s, 3H)

(b) Synthesis of 8-bromo-4-(4-methylpiperazin-1-yl)pyrido[2,3-e][1,2,4]triazolo[4,3-a]pyrazin-1(2H)-one

7-Bromo-2-hydrazinyl-3-(4-methylpiperazin-1-yl)pyrido[2,3-b]pyrazine (100 mg, 0.296 mmol) was dissolved in DMF (1.48 mL), and CDI (96.0 mg, 0.591 mmol) and TEA (0.0820 mL, 0.591 mmol) were added thereto. The reaction mixture was stirred at 65° C. for 1 hour and distilled under reduced pressure. The residue was purified by column chromatography (H₂O containing 0.1% formic acid=100) on reverse-phase silica and column chromatography (DCM:MeOH=80:20) on amine silica, and the fractions containing the product were collected and evaporated to obtain the solid compound, 8-bromo-4-(4-methylpiperazin-1-yl)pyrido[2,3-e][1,2,4]triazolo[4,3-a]pyrazin-1(2H)-one (5.80 mg, 5.4%) in white.

LC/MS ESI (+): 364 (M+1)

¹H NMR (400 MHz, DMSO-d₆) δ=13.16; (br s, 1H), 8.96; (d, J=2.3 Hz, 1H), 8.47; (d, J=2.2 Hz, 1H), 4.19; (br s, 4H), 2.49-2.47; (m, 4H), 2.24 (s, 3H)

### Example 7: Synthesis of 8-bromo-4-(4-methylpiperazin-1-yl)pyrido[2,3-e][1,2,4] triazolo[1,5-a]pyrazin-2(1H)-one

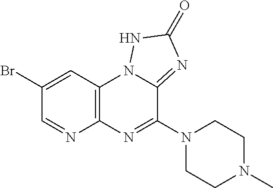 [SMILES: CN1CCN(c2nc3ncc(Br)cc3n3[nH]c(=O)nc23)CC1]

(a) Synthesis of 3-hydrazinyl-2-nitropyridine hydrofluoride

3-Fluoro-2-nitropyridine (5.56 g, 39.1 mmol) and hydrazine monohydrate (2.28 mL, 47.0 mmol) were dissolved in THF (78.0 mL) and stirred at room temperature for 13 hours. Hydrazine monohydrate (0.569 mL, 11.7 mmol) was added to the reaction mixture, and the resulting mixture was stirred at room temperature for 9 hours and distilled under reduced pressure. Et₂O was added to the residue, and this residue was filtered and dried under reduced pressure to obtain the solid compound, 3-hydrazinyl-2-nitropyridine hydrofluoride (6.55 g, 96%) in red.

LC/MS ESI (+): 155 (M+1)

¹H NMR (400 MHz, DMSO-d₆) δ=8.89; (br s, 1H), 8.12; (dd, J=8.7, 1.5 Hz, 1H), 7.78; (dd, J=3.9, 1.5 Hz, 1H), 7.61; (dd, J=8.7, 3.9 Hz, 1H), 4.68; (s, 2H)

(b) Synthesis of 2-(2-nitropyridine-3-yl)hydrazin-1-carboxamide

3-Hydrazinyl-2-nitropyridine (3.19 g, 20.7 mmol) and KOCN (2.52 g, 31.0 mmol) were dissolved in H₂O (20.7 mL), and 1N HCl (41.4 mL, 41.4 mmol) was added thereto. The reaction mixture was stirred at room temperature for 30 minutes, and H₂O was added thereto. The obtained solid was filtered and dried under reduced pressure to obtain the solid compound, 2-(2-nitropyridin-3-yl)hydrazin-1-carboxamide (3.79 g, 93%) in yellow.

LC/MS ESI (+): 198 (M+1)

¹H NMR (400 MHz, DMSO-d₆) δ=9.07; (s, 1H), 8.17; (s, 1H), 7.94; (dd, J=3.8, 1.7 Hz, 1H), 7.70-7.64; (m, 2H), 6.24; (br s, 2H)

(c) Synthesis of 1-(2-nitropyridin-3-yl)-1,2-dihydro-3H-1,2,4-triazol-3-one

2-(2-Nitropyridine-3-yl)hydrazin-1-carboxamide (3.79 g, 19.2 mmol) and p-toluene sulfonic acid (0.366 g, 1.92 mmol) were dissolved in triethoxymethane (80.0 mL, 481 mmol) and stirred at 120° C. for 1 hour. EtOH was added to the reaction mixture, and this mixture was filtered and dried under reduced pressure to obtain the solid compound, 1-(2-nitropyridin-3-yl)-1,2-dihydro-3H-1,2,4-triazol-3-one (3.05 g, 77%) in yellow.

LC/MS ESI (+): 208 (M+1)

¹H NMR (400 MHz, DMSO-d₆) δ=11.78; (br s, 1H), 8.86; (s, 1H), 8.63; (dd, J=4.6, 1.3 Hz, 1H), 8.52; (dd, J=8.1, 1.4 Hz, 1H), 8.02; (dd, J=8.2, 4.6 Hz, 1H)

(d) Synthesis of 2-(4-methoxybenzyl)-1-(2-nitropyridin-3-yl)-1,2-dihydro-3H -1,2,4-triazol-3-one

1-(2-Nitropyridin-3-yl)-1,2-dihydro-3H-1,2,4-triazol-3-one (3.05 g, 14.7 mmol) was dissolved in DMF (147 mL), and 60% NaH (0.589 g, 14.7 mmol) was slowly added thereto at 0° C. and stirred for 30 minutes. 4-Methoxybenzyl chloride (2.31 g, 14.7 mmol) was dissolved in DMF (147 mL), and slowly added to the reaction mixture at 0° C. and stirred at room temperature for 19 hours. Water was added to the reaction mixture, and this mixture was extracted with EtOAc (500 mL). The organic layer was washed with brine, dried over anhydrous Na₂SO₄, filtered and distilled under reduced pressure. The residue was purified by column chromatography (n-Hex:EtOAc=1:3) on silica, and the fractions containing the product were collected and evaporated to obtain the solid compound, 2-(4-methoxybenzyl)-1-(2-nitropyridin-3-yl)-1,2-dihydro-3 H-1,2,4-triazol-3-one (3.48 g, 72%) in orange.

LC/MS ESI (+): 328 (M+1)

¹H NMR (400 MHz, DMSO-d₆) δ=9.01; (s, 1H), 8.67; (dd, J=4.6, 1.3 Hz, 1H), 8.57; (dd, J=8.2, 1.5 Hz, 1H), 8.05; (dd, J=8.2, 4.6 Hz, 1H), 7.43-7.39; (m, 2H), 6.97-6.94; (m, 2H), 5.19; (s, 2H), 3.33; (s, 3H)

(e) Synthesis of 1-(2-aminopyridin-3-yl)-2-(4-methoxybenzyl)-1,2-dihydro-3H -1,2,4-triazol-3-one

2-(4-Methoxybenzyl)-1-(2-nitropyridin-3-yl)-1,2-dihydro-3H-1,2,4-triazol-3-one (3.48 g, 10.6 mmol) was dissolved in EtOH (53.2 mL) and H₂O (4.02 mL), and Fe (5.94 g, 106 mmol) and conc HCl (0.404 mL, 13.3 mmol) were added thereto at room temperature. The reaction mixture was refluxed for 1 hour and cooled to room temperature. MeOH was added to the reaction mixture, and this mixture was filtered with celite and distilled under reduced pressure. The residue was purified by column chromatography (EtOAc=100) on amine silica, and the fractions containing the product were collected and evaporated to obtain the solid compound, 1-(2-aminopyridin-3-yl)-2-(4-methoxybenzyl)-1,2-dihydro-3H-1,2,4-triazol-3-one (2.33 g, 74%) in yellow.

LC/MS ESI (+): 298 (M+1)

¹H NMR (400 MHz, DMSO-d₆) δ=8.62; (s, 1H), 8.06; (dd, J=4.9, 1.7 Hz, 1H), 7.64; (dd, J=7.7, 1.6 Hz, 1H), 7.44-7.41; (m, 2H), 6.98-6.95; (m, 2H), 6.69; (dd, J=7.6, 4.9 Hz, 1H), 6.28; (s, 2H), 5.25; (s, 2H), 3.77; (s, 3H)

(f) Synthesis of 1-(2-amino-5-bromopyridin-3-yl)-2-(4-methoxybenzyl)-1,2-dihydro-3H-1,2,4-triazol-3-one

1-(2-Aminopyridin-3-yl)-2-(4-methoxybenzyl)-1,2-dihydro-3H-1,2,4-triazol-3-one (2.33 g, 7.84 mmol) was dissolved in CH₃CN (78.0 mL), and N-bromosuccinimide (1.40 g, 7.84 mmol) was added thereto. The reaction mixture was stirred at 0° C. for 30 minutes and distilled under reduced pressure. The residue was purified by column chromatography (n-Hex:EtOAc=1:1) on silica, and the fractions containing the product were collected and evaporated to obtain the solid compound, 1-(2-amino-5-bromopyridin-3-yl)-2-(4-methoxybenzyl)-1,2-dihydro-3H-1,2,4-triazol-3-one (2.27 g, 77%) in ivory.

LC/MS ESI (+): 376 (M+1)

¹H NMR (400 MHz, DMSO-d₆) δ=8.68; (s, 1H), 8.15; (d, J=2.3 Hz, 1H), 7.95 (d, J=2.2 Hz, 1H), 7.44-7.40; (m, 2H), 6.98-6.95; (m, 2H), 6.56; (s, 2H), 5.26; (s, 2H), 3.33; (s, 3H)

(g) Synthesis of 8-bromopyrido[2,3-e][1,2,4]triazolo[1,5-a]pyrazin-2,4(1H,5H)-dione

1-(2-Amino-5-bromopyridin-3-yl)-2-(4-methoxybenzyl)-1,2-dihydro-3H-1,2,4-triazo 1-3-one (1.10 g, 2.92 mmol) was dissolved in 1,2-dichlorobenzene (29.2 mL), and CDI (1.90 g, 11.7 mmol) was added thereto. The reaction mixture was stirred at 170° C. for 1 hour and cooled to room temperature. H₂O was added to the reaction mixture, and the solid compound obtained by filtration was purified by column chromatography (H₂O containing 0.1% formic acid:CH₃CN=85:15) on reverse-phase silica, and the fractions containing the product were collected and evaporated to obtain the solid compound, 8-bromopyrido[2,3-e][1,2,4]triazolo[1,5-a]pyrazin-2,4(1H,5H)-dione (181 mg, 22%) in dark brown.

LC/MS ESI (+): 282 (M+1)

¹H NMR (400 MHz, DMSO-d₆) δ=12.92; (br s, 1H), 12.54; (br s, 1H), 8.56; (d, J=2.2 Hz, 1H), 8.35; (d, J=2.1 Hz, 1H)

(h) Synthesis of 8-bromo-4-(4-methylpiperazin-1-yl)pyrido[2,3-e][1,2,4]triazolo[1,5-a]pyrazin-2(1H)-one

8-Bromopyrido[2,3-e][1,2,4]triazolo[1,5-a]pyrazin-2,4(1H,5H)-dione (50.0 mg, 0.177 mmol) was dissolved in POCl₃ (0.496 mL, 5.32 mmol) and stirred at 100° C. for 3.5 hours. The reaction mixture was cooled to 0° C., and 1-methylpiperazine (2.96 mL, 26.6 mmol) was slowly added thereto. The reaction mixture was stirred at room temperature for 1 hour and distilled under reduced pressure. The residue was purified by column chromatography (H₂O containing 0.1% formic acid:CH₃CN=80:20) on reverse-phase silica, and the fractions containing the product were collected and evaporated to obtain 8-bromo-4-(4-methylpiperazin-1-yl)pyrido[2,3-e][1,2,4]triazolo[1,5-a]pyrazin-2(1H)-one (7.60 mg, 12%) in brown.

LC/MS ESI (+): 364 (M+1)

¹H NMR (400 MHz, DMSO-d₆) δ=8.59; (br s, 1H), 8.32; (br s, 1H), 4.22; (br s, 4H), 2.51-2.47; (m, 4H), 2.18; (br s, 3H)

### Example 8: Synthesis of 8-bromo-7-chloro-4-(4-methylpiperazin-1-yl)imidazo[1,2-a]pyrido[2,3-e]pyrazin-2(1H)-one

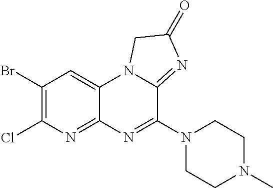 [SMILES: CN1CCN(C2=Nc3nc(Cl)c(Br)cc3N3CC(=O)N=C23)CC1]

(a) Synthesis of 5-bromo-6-chloro-3-nitropyridin-2-amine

6-Chloro-3-nitropyridin-2-amine (1.00 g, 5.76 mmol) was dissolved in DMF (19.2 mL), and N-bromosuccinimide (1.13 g, 6.34 mmol) was added thereto at room temperature. The reaction mixture was stirred at room temperature for 3 hours, and H₂O (19.2 mL) was added thereto. The obtained solid was filtered and dried under reduced pressure dried to obtain the solid compound, 5-bromo-6-chloro-3-nitropyridin-2-amine (1.27 g, 87%) in yellow.

LC/MS ESI (+): 252 (M+1)

¹H NMR (400 MHz, DMSO-d₆) δ=8.64; (s, 1H), 8.51-8.18; (m, 2H)

(b) Synthesis of 5-bromo-6-chloropyridin-2,3-diamine

5-Bromo-6-chloro-3-nitropyridin-2-amine (1.27 g, 5.03 mmol) was dissolved in EtOH (4.02 mL) and H₂O (1.01 mL), and Fe (2.81 g, 50.3 mmol) and conc HCl (0.0760 mL, 2.52 mmol) were added thereto at room temperature. The reaction mixture was stirred at 100° C. for 1 hour and cooled to room temperature. The reaction mixture was filtered with celite and distilled under reduced pressure. The residue was purified by column chromatography (DCM:MeOH=20:1) on silica, and the fractions containing the product were collected and evaporated to obtain the solid compound, 5-bromo-6-chloropyridin-2,3-diamine (1.03 g, 92%) in grey.

LC/MS ESI (+): 222 (M+1)

¹H NMR (400 MHz, DMSO-d₆) δ=6.93; (s, 1H), 6.06; (s, 2H), 5.09; (s, 2H)

(c) Synthesis of 7-bromo-6-chloro-1,4-dihydropyrido[2,3-b]pyrazin-2,3-dione

5-Bromo-6-chloropyridin-2,3-diamine (1.03 g, 4.63 mmol) was dissolved in diethyl oxalate (9.26 mL, 4.63 mmol) and stirred at 130° C. for 20 hours. The reaction mixture was cooled to room temperature. The obtained solid was filtered, washed with Et₂O and dried under reduced pressure dried to obtain the solid compound, 7-bromo-6-chloro-1,4-dihydropyrido[2,3-b]pyrazin-2,3-dione (1.19 g, 93%) in brown.

LC/MS ESI (+): 276 (M+1)

¹H NMR (400 MHz, DMSO-d₆) δ=12.65; (s, 1H), 12.09; (s, 1H), 7.67; (s, 1H)

(d) Synthesis of 7-bromo-2,3,6-trichloropyrido[2,3-b]pyrazine

To 7-bromo-6-chloro-1,4-dihydropyrido[2,3-b]pyrazin-2,3-dione (506 mg, 1.83 mmol) POCl₃ (6.10 mL, 1.83 mmol) was added, and this mixture was stirred at 130° C. for 15 hours. The reaction mixture was cooled to 0° C., and ice water (12.0 mL) was added thereto. The obtained solid was filtered, washed with H₂O and dried under reduced pressure dried to obtain the solid compound, 7-bromo-2,3,6-trichloropyrido[2,3-b]pyrazine (417 mg, 73%) in brown.

LC/MS ESI (+): 312 (M+1)

¹H NMR (400 MHz, DMSO-d₆) δ=9.20; (s, 1H)

(e) Synthesis of 7-bromo-2,6-dichloro-3-(4-methylpiperazin-1-yl)pyrido[2,3-b]pyrazine

7-Bromo-2,3,6-trichloropyrido[2,3-b]pyrazine (417 mg, 1.33 mmol) was dissolved in

DMF (4.44 mL), and 1-methylpiperazine (0.295 mL, 2.66 mmol) was slowly added thereto at 0° C. The reaction mixture was stirred at 25° C. for 1 hour, H₂O (5.00 mL) was added thereto and extracted with EtOAc (10.0 mL). The organic layer was washed with H₂O and brine, dried over anhydrous MgSO₄, filtered and distilled under reduced pressure. The residue was purified by column chromatography (DCM:MeOH=20:1) on silica, and the fractions containing the product were collected and evaporated to obtain the solid compound, 7-bromo-2,6-dichloro-3-(4-methylpiperazin-1-yl)pyrido[2,3-b]pyrazine (195 mg, 39%) in brown.

LC/MS ESI (+): 376 (M+1)

¹H NMR (400 MHz, CDCl₃) δ=8.41; (s, 1H), 3.84-3.82; (m, 4H), 2.64-2.62; (m, 4H), 2.37; (s, 3H)

(f) Synthesis of N-(7-bromo-6-chloro-3-(4-methylpiperazin-1-yl)pyrido[2,3-b]pyrazin-2-yl)-2-hydroxyacetamide

7-Bromo-2,6-dichloro-3-(4-methylpiperazin-1-yl)pyrido[2,3-b]pyrazine (195 mg, 0.517 mmol) and glycolamide (46.6 mg, 0.621 mmol) were dissolved in DMF (5.17 mL), and anhydrous K₂CO₃ (107 mg, 0.776 mmol) was added thereto at room temperature. The reaction mixture was stirred at 70° C. for 1 hour and distilled under reduced pressure. The residue was purified by column chromatography (H₂O containing 0.1% formic acid:CH₃CN=70:30) on reverse-phase silica and column chromatography (DCM:MeOH=20:1) on silica, and the fractions containing the product were collected and evaporated to obtain the solid compound, N-(7-bromo-6-chloro-3-(4-methylpiperazin-1-yl)pyrido[2,3-b]pyrazin-2-yl)-2-hydroxyacetamide (115 mg, 54%) in brown.

LC/MS ESI (+): 415 (M+1)

¹H NMR (400 MHz, DMSO-d₆) δ=8.35; (s, 1H), 7.61; (s, 1H), 7.31; (s, 1H), 4.93; (s, 2H), 3.93-3.91; (m, 4H), 2.48-2.46; (m, 4H), 2.23; (s, 3H)

(g) Synthesis of 8-bromo-7-chloro-4-(4-methylpiperazin-1-yl)imidazo[1,2-a]pyrido[2,3-e]pyrazin-2(1H)-one

N-(7-bromo-6-chloro-3-(4-methylpiperazin-1-yl)pyrido[2,3-b]pyrazin-2-yl)-2-hydroxyacetamide (115 mg, 0.277 mmol) was dissolved in DMF (5.53 mL), and methane-sulfonyl chloride (0.645 mL, 8.30 mmol) and TEA (1.16 mL, 8.30 mmol) were added thereto at room temperature. The reaction mixture was stirred at 80° C. for 2 hours and distilled under reduced pressure. The residue was purified by column chromatography (H₂O containing 0.1% formic acid:CH₃CN=60:40) on reverse-phase silica and column chromatography (DCM:MeOH=9:1) on silica, and the fractions containing the product were collected and evaporated to obtain the solid compound, 8-bromo-7-chloro-4-(4-methylpiperazin-1-yl)imidazo[1,2-a]pyrido[2,3-e]pyrazin-2(1H)-one (51.0 mg, 46%) in yellow.

LC/MS ESI (+): 397 (M+1)

¹H NMR (400 MHz, DMSO-d₆) δ=8.50; (s, 1H), 5.39; (s, 2H), 3.86-3.84; (m, 4H), 2.48-2.47; (m, 4H), 2.23; (s, 3H)

### Example 9: Synthesis of 8-bromo-7-fluoro-4-(4-methylpiperazin-1-yl)imidazo[1.2-a]pyrido[2,3-e]pyrazin-2(1H)-one

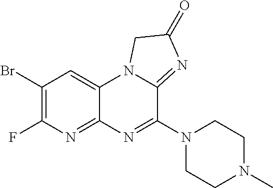 [SMILES: CN1CCN(C2=Nc3nc(F)c(Br)cc3N3CC(=O)N=C23)CC1]

8-Bromo-7-chloro-4-(4-methylpiperazin-1-yl)imidazo[1,2-a]pyrido[2,3-e]pyrazin-2(1H)-one (80.0 mg, 0.201 mmol) obtained in Example 8 was dissolved in DMSO (2.01 mL), and CsF (92.0 mg, 0.604 mmol) was added thereto at room temperature. The reaction mixture was stirred at 90° C. for 1 hour and purified by column chromatography (H₂O containing 0.1% TFA:CH₃CN=65:35) on reverse-phase silica, column chromatography (DCM:MeOH=9:1) on silica and column chromatography (DCM:MeOH=100:1) on amine silica. The fractions containing the product were collected and evaporated to obtain the solid compound, 8-bromo-7-fluoro-4-(4-methylpiperazin-1-yl)imidazo[1,2-a]pyrido[2,3-e]pyrazin-2(1H)-one (5.50 mg, 7.2%) in yellow.

LC/MS ESI (+): 381 (M+1)

¹H NMR (400 MHz, DMSO-d₆) δ=8.57; (d, J=8.8 Hz, 1H), 5.38; (s, 2H), 3.85-3.83; (m, 4H), 2.49-2.47; (m, 4H), 2.23; (s, 3H)

### Example 10: Synthesis of 4-(4-methylpiperazin-1-yl)-8-nitroimidazo[1,2-a]quinoxalin-2(1H)-one

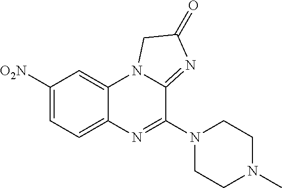 [SMILES: CN1CCN(C2=Nc3ccc([N+](=O)[O-])cc3N3CC(=O)N=C23)CC1]

(a) Synthesis of 2-hydroxy-N-(3-(4-methylpiperazin-1-yl)-7-nitroquinoxalin-2-yl)acetamide

3-Chloro-2-(4-methylpiperazin-1-yl)-6-nitroquinoxaline (490 mg, 1.59 mmol) and glycolamide (143 mg, 1.91 mmol) were dissolved in DMF (15.9 mL), and anhydrous K₂CO₃ (330 mg, 2.39 mmol) was added thereto at room temperature. The reaction mixture was stirred at 70° C. for 1 hour and distilled under reduced pressure. The residue was purified by column chromatography (H₂O containing 0.1% formic acid:CH₃CN=70:30) on reverse-phase silica and column chromatography (DCM:MeOH=20:1) on silica, and the fractions containing the product were collected and evaporated to obtain the solid compound, 2-hydroxy-N-(3-(4-methylpiperazin-1-yl)-7-nitroquinoxalin-2-yl)acetamide (345 mg, 63%) in brown.

LC/MS ESI (+): 347 (M+1)

¹H NMR (400 MHz, DMSO-d₆) δ=8.32; (d, J=2.6 Hz, 1H), 8.22; (dd, J=9.0, 2.6

Hz, 1H), 7.72; (d, J=9.0 Hz, 1H), 7.63; (br s, 1H), 7.30; (br s, 1H), 4.96; (s, 2H), 3.95-3.93; (m, 4H), 2.50-2.48; (m, 4H), 2.24; (s, 3H)

(b) Synthesis of 4-(4-methylpiperazin-1-yl)-8-nitroimidazo[1,2-a]quinoxalin-2(1H)-one

2-Hydroxy-N-(3-(4-methylpiperazin-1-yl)-7-nitroquinoxalin-2-yl)acetamide (345 mg, 0.996 mmol) was dissolved in DMF (9.96 mL), and methanesulfonyl chloride (2.32 mL, 29.9 mmol) and TEA (4.17 mL, 29.9 mmol) were added thereto at room temperature. The reaction mixture was stirred at 80° C. for 2 hours and distilled under reduced pressure. The residue was purified by column chromatography (H₂O containing 0.1% formic acid:CH₃CN=60:40) on reverse-phase silica and column chromatography (DCM:MeOH=20:1) on silica, and the fractions containing the product were collected and evaporated to obtain the solid compound, 4-(4-methylpiperazin-1-yl)-8-nitroimidazo[1,2-a]quinoxalin-2(1H)-one (150 mg, 46%) in yellow.

LC/MS ESI (+): 329 (M+1)

¹H NMR (400 MHz, DMSO-d₆) δ=8.36; (d, J=2.3 Hz, 1H), 8.20; (dd, J=9.0, 2.4 Hz, 1H), 7.69; (d, J=9.0 Hz, 1H), 5.36; (s, 2H), 3.80-3.78; (m, 4H), 2.43-2.41; (m, 4H), 2.16; (s, 3H)

### Example 11: Synthesis of 8-amino-4-(4-methylpiperazin-1-yl)imidazo[1,2-a]quinoxalin-2(1H)-one

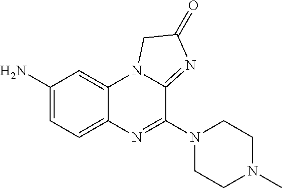 [SMILES: CN1CCN(C2=Nc3ccc(N)cc3N3CC(=O)N=C23)CC1]

4-(4-Methylpiperazin-1-yl)-8-nitroimidazo[1,2-a]quinoxalin-2(1H)-one (105 mg, 0.320 mmol) obtained in Example 10 was dissolved in EtOH (2.56 mL) and H₂O (0.640 mL), and Fe (179 mg, 3.20 mmol) and conc HCl (0.00486 mL, 0.160 mmol) were added thereto at room temperature. The reaction mixture was stirred at 100° C. for 1 hour and cooled to room temperature. The reaction mixture was filtered with celite and distilled under reduced pressure. The residue was purified by column chromatography (DCM:MeOH=10:1) on silica and column chromatography (DCM:MeOH=10:1) on amine silica, and the fractions containing the product were collected and evaporated to obtain the solid compound, 8-amino-4-(4-methylpiperazin-1-yl)imidazo[1,2-a]quinoxalin-2(1H)-one (85.0 mg, 89%) in yellow.

LC/MS ESI (+): 299 (M+1)

¹H NMR (400 MHz, DMSO-d₆) δ=7.44; (d, J=8.8 Hz, 1H), 6.93; (dd, J=8.7, 1.8 Hz, 1H), 6.77; (d, J=1.7 Hz, 1H), 5.54; (s, 2H), 5.32; (s, 2H), 3.41-3.39; (m, 4H), 2.49-2.47; (m, 4H), 2.23; (s, 3H)

### Example 12: Synthesis of 8-bromo-4-(4-methylpiperazin-1-yl)imidazo[1,2-a]quinoxalin-2(1H)-one

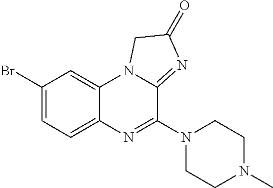 [SMILES: CN1CCN(C2=Nc3ccc(Br)cc3N3CC(=O)N=C23)CC1]

(a) Synthesis of 3-chloro-2-(4-methylpiperazin-1-yl)-6-nitroquinoxaline

2,3-Dichloro-6-nitroquinoxaline (2.40 g, 9.83 mmol) was dissolved in DCM (98.0 mL), and 1-methylpiperazine (2.74 mL, 24.6 mmol) was added thereto. The reaction mixture was stirred at room temperature for 2 hours. The reaction mixture was filtered to remove insoluble materials. Saturated NaHCO₃ aqueous solution was added to the organic layer, and this layer was extracted with DCM (250 mL). The organic layer was washed with H₂O and brine, dried over anhydrous Na₂SO₄, filtered and distilled under reduced pressure. The residue was purified by column chromatography (n-Hex:EtOAc=2:1) on amine silica, and the fractions containing the product were collected and evaporated to obtain the solid compound, 3-chloro-2-(4-methylpiperazin-1-yl)-6-nitroquinoxaline (2.06 g, 68%) in yellow.

LC/MS ESI (+): 308 (M+1)

¹H NMR (400 MHz, CDCl₃) δ=8.75; (s, 1H), 8.41; (d, J=9.0 Hz, 1H), 7.85; (d, J=9.2 Hz, 1H), 3.79; (br s, 4H), 2.67-2.63; (m, 4H), 2.39; (s, 3H)

(b) Synthesis of 2-hydroxy-N -(3-(4-methylpiperazin-1-yl)-7-nitroquinoxalin-2-yl)acetamide

3-Chloro-2-(4-methylpiperazin-1-yl)-6-nitroquinoxaline (2.06 g, 6.69 mmol) and glycolamide (0.603 g, 8.03 mmol) were dissolved in DMF (66.9 mL), and anhydrous K₂CO₃ (1.39 g, 10.0 mmol) was added thereto at room temperature. The reaction mixture was stirred at 70° C. for 2 hours. H₂O was added to the reaction mixture, and this mixture was extracted with EtOAc (160 mL). The organic layer was washed with brine, dried over anhydrous Na₂SO₄, filtered and distilled under reduced pressure. The residue was purified by column chromatography (DCM:MeOH=93:7) on silica, and the fractions containing the product were collected and evaporated to obtain the solid compound, 2-hydroxy-N-(3-(4-methylpiperazin-1-yl)-7-nitroquinoxalin-2-yl)acetamide (1.08 g, 47%) in brown.

LC/MS ESI (+): 347 (M+1)

¹H NMR (400 MHz, DMSO-d₆) δ=8.31; (d, J=2.6 Hz, 1H), 8.22; (dd, J=9.0, 2.5 Hz, 1H), 7.72; (d, J=9.0 Hz, 1H), 7.63; (br s, 1H), 7.30; (br s, 1H), 4.96; (s, 2H), 3.95-3.93; (m, 4H), 2.50-2.48; (m, 4H), 2.23; (s, 3H)

(c) Synthesis of 4-(4-methylpiperazin-1-yl)-8-nitroimidazo[1,2-a]quinoxalin-2(1H)-one

2-Hydroxy-N-(3-(4-methylpiperazin-1-yl)-7-nitroquinoxalin-2-yl)acetamide (1.08 g, 3.12 mmol) was dissolved in DMF (31.2 mL), and TEA (4.35 mL, 31.2 mmol) and methanesulfonyl chloride (2.43 mL, 31.2 mmol) were added thereto. The resulting mixture was stirred at 80° C. for 3 hours. Saturated NaHCO₃ aqueous solution was added to the reaction mixture, and this mixture was extracted with EtOAc (170 mL). The organic layer was washed with H₂O and brine, dried over anhydrous Na₂SO₄, and filtered and distilled under reduced pressure. The residue was purified by column chromatography (DCM:MeOH=90:10) on silica, and the fractions containing the product were collected and evaporated to obtain the solid compound, 4-(4-methylpiperazin-1-yl)-8-nitroimidazo[1,2-a]quinoxalin-2(1H)-one (539 mg, 53%) in brown.

LC/MS ESI (+): 329 (M+1)

¹H NMR (400 MHz, DMSO-d₆) δ=8.43; (d, J=2.4 Hz, 1H), 8.27; (dd, J=9.0, 2.6 Hz, 1H), 7.76; (d, J=9.0 Hz, 1H), 5.44; (s, 2H), 3.88-3.86; (m, 4H), 2.50-2.47; (m, 4H), 2.24; (s, 3H)

(d) Synthesis of 8-amino-4-(4-methylpiperazin-1-yl)imidaz[1,2-a]quinoxalin-2(1H)-one

4-(4-Methylpiperazin-1-yl)-8-nitroimidazo[1,2-a]quinoxalin-2(1H)-one (539 mg, 1.64 mmol) was dissolved in EtOH (13.1 mL) and H₂O (3.28 mL), and Fe (917 mg, 16.4 mmol) and conc HCl (0.0249 mL, 0.821 mmol) were added thereto. The reaction mixture was refluxed for 2 hours and cooled to room temperature. MeOH was added to the reaction mixture, and this mixture was filtered with celite and distilled under reduced pressure. Saturated NaHCO₃ aqueous solution was added to the residue, and this mixture was extracted with EtOAc (80.0 mL). The organic layer was washed with H₂O and brine, dried over anhydrous Na₂SO₄, filtered and distilled under reduced pressure. The residue was purified by column chromatography (n-Hex:EtOAc=1:2) on amine silica, and the fractions containing the product were collected and evaporated to obtain the solid compound, 8-amino-4-(4-methylpiperazin-1-yl)imidazo[1,2-a]quinoxalin-2(1H)-one (312 mg, 64%) in yellow.

LC/MS ESI (+): 299 (M+1)

¹H NMR (400 MHz, DMSO-d₆) δ=7.43; (d, J=8.7 Hz, 1H), 6.93; (dd, J=8.8, 2.4 Hz, 1H), 6.77; (d, J=2.3 Hz, 1H), 5.54; (s, 2H), 5.32; (s, 2H), 3.41-3.38; (m, 4H), 2.49-2.46; (m, 4H), 2.23; (s, 3H)

(e) Synthesis of 8-bromo-4-(4-methylpiperazin-1-yl)imidazo[1,2-a]quinoxalin-2(1H)-one

8-Amino-4-(4-methylpiperazin-1-yl)imidazo[1,2-a]quinoxalin-2(1H)-one (200 mg, 0.670 mmol) was dissolved in CH₃CN (4.47 mL), and tert-butyl nitrite (0.177 mL, 1.34 mmol) was added thereto. This mixture was stirred at room temperature for 20 minutes. CuBr₂ (150 mg, 0.670 mmol) was added to the reaction mixture, and this mixture was stirred at room temperature for 2 hours. Tert-butyl nitrite (0.177 mL, 1.34 mmol) was added to the reaction mixture, and this mixture was stirred at room temperature for 1.5 hours. The reaction mixture was purified by column chromatography (H₂O containing 0.1% formic acid:CH₃CN=60:40) on reverse-phase silica and column chromatography (n-Hex:EtOAc=1:1) on amine silica, and the fractions containing the product were collected and evaporated to obtain the solid compound, 8-bromo-4-(4-methylpiperazin-1-yl)imidazo[1,2-a]quinoxalin-2(1H)-one (31.0 mg, 13%) in white.

LC/MS ESI (+): 362 (M+1)

¹H NMR (400 MHz, DMSO-d₆) δ=7.88; (d, J=1.8 Hz, 1H), 7.68-7.61; (m, 2H), 5.39; (s, 2H), 3.68-3.65; (m, 4H), 2.50-2.47; (m, 4H), 2.24; (s, 3H)

### Example 13: Synthesis of 8-bromo-4-(3-(methylamino)azetidin-1-yl)imidazo[1,2-a]quinoxalin-2(1H)-one

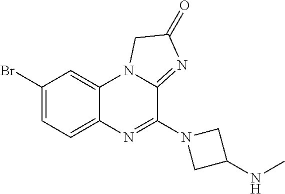 [SMILES: CNC1CN(C2=Nc3ccc(Br)cc3N3CC(=O)N=C23)C1]

(a) Synthesis of tert-butyl (1-(3-chloro-6-nitroquinoxalin-2-yl)azetidin-3-yl)(methyl)carbamate

2,3-Dichloro-6-nitroquinoxaline (2.40 g, 9.83 mmol) was dissolved in DCM (98.0 mL), and TEA (2.74 mL, 19.7 mmol) and tert-butyl azetidin-3-yl-(methyl)carbamate hydrochloride (4.38 g, 19.7 mmol) were added thereto. This mixture was stirred at room temperature for 2 hours. Saturated NaHCO₃ aqueous solution was added to the reaction mixture, and this mixture was extracted with DCM (120 mL). The organic layer was washed with brine, dried over anhydrous Na₂SO₄, filtered and distilled under reduced pressure. The residue was purified by column chromatography (n-Hex:EtOAc=4:1) on silica, and the fractions containing the product were collected and evaporated to obtain the solid compound, tert-butyl (1-(3-chloro-6-nitroquinoxalin-2-yl)azetidin-3-yl)(methyl)carbamate (1.66 g, 43%) in yellow.

LC/MS ESI (+): 394 (M+1)

¹H NMR (400 MHz, CDCl₃) δ=8.69; (s, 1H), 8.36; (d, J=9.0 Hz, 1H), 7.73; (d, J=9.0 Hz, 1H), 5.07; (br s, 1H), 4.74; (br s, 2H), 4.55; (br s, 2H), 3.00; (s, 3H), 1.49; (s, 9H)

(b) Synthesis of tert-butyl (1-(3-(2-hydroxyacetamido)-6-nitroquinoxalin-2-yl)azetidin-3-yl)(methyl)carbamate

Tert-butyl (1-(3-chloro-6-nitroquinoxalin-2-yl)azetidin-3-yl)(methyl)carbamate (1.66 g, 4.22 mmol) and glycolamide (0.380 g, 5.06 mmol) were dissolved in DMF (42.2 mL), and anhydrous K₂CO₃ (0.874 g, 6.32 mmol) was added thereto. The reaction mixture was stirred at 70° C. for 2 hours and cooled to room temperature. Water was added to the reaction mixture, and this mixture was extracted with EtOAc (110 mL). The organic layer was washed with brine, dried over anhydrous Na₂SO₄, filtered and distilled under reduced pressure. The residue was purified by column chromatography (EtOAc=100) on silica, and the fractions containing the product were collected and evaporated to obtain the solid compound, tert-butyl (1-(3-(2-hydroxyacetamido)-6-nitroquinoxalin-2-yl)azetidin-3-yl)(methyl)carbamate (988 mg, 54%) in brown.

LC/MS ESI (+): 433 (M+1)

¹H NMR (400 MHz, DMSO-d₆) δ=8.29; (d, J=2.6 Hz, 1H), 8.18; (dd, J=9.0, 2.6 Hz, 1H), 7.65; (d, J=9.0 Hz, 1H), 7.58; (s, 1H), 7.35; (s, 1H), 5.07-4.15; (m, 7H), 2.91; (s, 3H), 1.42; (s, 9H)

(c) Synthesis of tert-butyl methyl(1-(8-nitro-2-oxo-1,2-dihydroimidazo[1,2-a]quinoxalin-4-yl)azetidin-3-yl)carbamate

Tert-butyl (1-(3-(2-hydroxyacetamido)-6-nitroquinoxalin-2-yl)azetidin-3-yl)(methyl)carbamate (899 mg, 2.08 mmol) was dissolved in DMF (20.8 mL), and TEA (1.74 mL, 12.5 mmol) and methanesulfonyl chloride (0.583 mL, 7.48 mmol) were added thereto. This mixture was stirred at 50° C. for 22 hours. Saturated NaHCO₃ aqueous solution was added to the reaction mixture, and this mixture was extracted with EtOAc (120 mL). The organic layer was washed with H₂O and brine, dried over anhydrous Na₂SO₄, filtered and distilled under reduced pressure. The residue was purified by column chromatography (n-Hex:EtOAc=3:2) on silica, and the fractions containing the product were collected and evaporated to obtain the solid compound, tert-butyl methyl(1-(8-nitro-2-oxo-1,2-dihydroimidazo[1,2-a]quinoxalin-4-yl)azetidin-3-yl)carbamate (691 mg, 80%) in yellow.

LC/MS ESI (+): 415 (M+1)

¹H NMR (400 MHz, DMSO-d₆) δ=8.39; (d, J=2.7 Hz, 1H), 8.24; (dd, J=9.0, 2.7 Hz, 1H), 7.70; (d, J=9.0 Hz, 1H), 5.40; (s, 2H), 5.12-4.15; (m, 5H), 2.90; (s, 3H), 1.42; (s, 9H)

(d) Synthesis of tert-butyl (1-(8-amino-2-oxo-1,2-dihydroimidazo[1,2-a]quinoxalin-4-yl)azetidin-3-yl)(methyl)carbamate

Tert-butyl methyl(1-(8-nitro-2-oxo-1,2-dihydroimidazo[1,2-a]quinoxalin-4-yl)azetidin-3-yl)carbamate (629 mg, 1.52 mmol) was dissolved in EtOH (12.1 mL) and H₂O (3.04 mL), and Fe (848 mg, 15.2 mmol) and conc HCl (0.0231 mL, 0.759 mmol) were added thereto. The reaction mixture was refluxed for 1 hour 40 minutes and cooled to room temperature. After addition of MeOH, the reaction mixture was filtered with celite and distilled under reduced pressure. The residue was purified by column chromatography (n-Hex:EtOAc=1:1) on amine silica, and the fractions containing the product were collected and evaporated to obtain the solid compound, tert-butyl (1-(8-amino-2-oxo-1,2-dihydroimidazo[1,2-a]quinoxalin-4-yl)azetidin-3-yl)(methyl)carbamate (436 mg, 75%) in yellow.

LC/MS ESI (+): 385 (M+1)

¹H NMR (400 MHz, DMSO-d₆) δ=7.37; (d, J=8.8 Hz, 1H), 6.88; (d, J=9.7 Hz, 1H), 6.76; (s, 1H), 5.37; (s, 2H), 5.28; (s, 2H), 4.81; (br s, 1H), 4.30; (t, J=8.5 Hz, 2H), 4.17-4.13; (m, 2H), 2.87; (s, 3H), 1.41; (s, 9H)

(e) Synthesis of tert-butyl (1-(8-bromo-2-oxo-1,2-dihydroimidazo[1,2-a]quinoxalin-4-yl)azetidin-3-yl)(methyl)carbamate

Tert-butyl (1-(8-amino-2-oxo-1,2-dihydroimidazo[1,2-a]quinoxalin-4-yl)azetidin-3-yl)(methyl)carbamate (408 mg, 1.06 mmol) was dissolved in CH₃CN (7.08 mL), and tert-butyl nitrite (0.281 mL, 2.12 mmol) was added thereto. This mixture was stirred at room temperature for 20 minutes. After addition of CuBr₂ (237 mg, 1.06 mmol), the reaction mixture was stirred at room temperature for 1 hour.

The reaction mixture was purified by column chromatography (H₂O containing 0.1% formic acid:CH₃CN =35:65) on reverse-phase silica and column chromatography (n-Hex:EtOAc=7:2) on silica, and the fractions containing the product were collected and evaporated to obtain the solid compound, tert-butyl (1-(8-bromo-2-oxo-1,2-dihydroimidazo[1,2-a]quinoxalin-4-yl)azetidin-3-yl)(methyl)carbamate (61.0 mg, 13%) in white.

LC/MS ESI (+): 448 (M+1)

¹H NMR (400 MHz, CDCl₃) δ=7.85; (m, 1H), 7.54; (m, 2H), 5.13; (s, 2H), 5.09-4.83; (m, 1H), 4.55; (t, J=9.1 Hz, 2H), 4.36; (dd, J=10.1, 6.2 Hz, 2H), 2.97; (s, 3H), 1.48; (s, 9H)

(f) Synthesis of 8-bromo-4-(3-(methylamino)azetidin-1-yl)imidazo[1,2-a]quinoxalin-2(1H)-one

Tert-butyl (1-(8-bromo-2-oxo-1,2-dihydroimidazo[1,2-a]quinoxalin-4-yl)azetidin-3-yl)(methyl)carbamate (61.0 mg, 0.136 mmol) was dissolved in DCM (0.580 mL), and TFA (0.335 mL) was added thereto. The reaction mixture was stirred at room temperature for 21 hours, and DIPEA (0.760 mL) was added at 0° C. and stirred for 30 minutes. The reaction mixture was purified by column chromatography (EtOAc=100) on amine silica, and the fractions containing the product were collected and evaporated to obtain the solid compound, 8-bromo-4-(3-(methylamino)azetidin-1-yl)imidazo[1,2-a]quinoxalin-2(1H)-one (27.0 mg, 54%) in white.

LC/MS ESI (+): 348 (M+1)

¹H NMR (400 MHz, CDCl₃) δ=7.83; (dd, J=1.8, 0.7 Hz, 1H), 7.53-7.52; (m, 2H), 5.12; (s, 2H), 4.52; (dd, J=9.5, 7.7 Hz, 2H), 4.06; (dd, J=10.1, 4.6 Hz, 2H), 3.76-3.70; (m, 1H), 2.46; (s, 3H)

### Example 14: Synthesis of 9-bromo-5-(4-methylpiperazin-1-yl)-1H -[1,2,4]triazino[43-a]quinoxalin-2(3H)-one

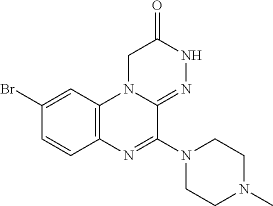 [SMILES: CN1CCN(C2=Nc3ccc(Br)cc3N3CC(=O)NN=C23)CC1]

(a) Synthesis of 6-bromo-1,4-dihydroquinoxalin-2,3-dione

4-Bromobenzene-1,2-diamine (3.68 g, 19.7 mmol) was dissolved in diethyl oxalate (85.0 mL, 620 mmol) and stirred at 120° C. for 3 hours. The reaction mixture was cooled to room temperature. After addition of EtOH, the obtained solid was dried under reduced pressure to obtain the solid compound, 6-bromo-1,4-dihydroquinoxalin-2,3-dione (4.64 g, 98%) in brown.

LC/MS ESI (+): 241 (M+1)

¹H NMR (400 MHz, DMSO-d₆) δ=11.99; (s, 1H), 11.96; (s, 1H), 7.26-7.24; (m, 2H), 7.07-7.05; (m, 1H)

(b) Synthesis of 6-bromo-2,3-dichloroquinoxaline

6-Bromo-1,4-dihydroquinoxalin-2,3-dione (4.64 g, 19.3 mmol) was dissolved in POCl₃ (96.4 mL, 1.03 mol), and N,N-dimethylaniline (3.52 mL, 27.8 mmol) was added thereto. The reaction mixture was stirred at 150° C. for 67 hours and cooled to 0° C. After slow addition of H₂O, the obtained solid was washed with H₂O, and the filtrate was dried under reduced pressure to obtain the solid compound, 6-bromo-2,3-dichloroquinoxaline (3.53 g, 66%) in yellow.

¹H NMR (400 MHz, DMSO-d₆) δ=8.40; (d, J=2.0 Hz, 1H), 8.12-8.04; (m, 2H)

(c) Synthesis of 6-bromo-2-chloro-3-hydrazinylquinoxaline

6-Bromo-2,3-dichloroquinoxaline (724 mg, 2.60 mmol) was dissolved in EtOH (26.0 mL), and hydrazine monohydrate (0.190 mL, 3.91 mmol) was added thereto. The reaction mixture was stirred at room temperature for 4 hours and distilled under reduced pressure. DCM was added to the residue, and the obtained solid was filtrated and washed with DCM. The filtrate was dried under reduced pressure dried. The filtrated solid was purified by column chromatography (n-Hex:EtOAc=3:2) on silica, and the fractions containing the product were collected and evaporated to obtain the solid compound, 6-bromo-2-chloro-3-hydrazinylquinoxaline (179 mg, 25%) in yellow.

LC/MS ESI (+): 273 (M+1)

¹H NMR (400 MHz, DMSO-d₆) δ=9.17; (br s, 1H), 7.83; (s, 1H), 7.69; (d, J=8.7 Hz, 1H), 7.52; (d, J=8.7 Hz, 1H), 4.70; (br s, 2H)

(d) Synthesis of (Z)-N″-(7-bromo-3-chloroquinoxalin-2(1H)-ylidene)-2-chloroacetohydrazide

6-Bromo-2-chloro-3-hydrazinylquinoxaline (179 mg, 0.654 mmol) was dissolved in DMF (6.54 mL), and chloroacetyl chloride (0.0524 mL, 0.654 mmol) was added there to. The reaction mixture was stirred at room temperature for 40 minutes. The reaction mixture was purified by column chromatography (H₂O containing 0.1% formic acid:CH₃CN=50:50) on reverse-phase silica, and the fractions containing the product were collected and evaporated to obtain the solid compound, (Z)-N″-(7-bromo-3-chloroquinoxalin-2(1H)-ylidene)-2-chloroacetohydrazide (215 mg, 94%) in ivory.

LC/MS ESI (+): 349 (M+1)

¹H NMR (400 MHz, DMSO-d₆) δ=10.53; (br s, 1H), 9.82; (br s, 1H), 7.88; (s, 1H), 7.80; (d, J=8.8 Hz, 1H), 7.67; (d, J=8.9 Hz, 1H), 4.29; (s, 2H)

(e) Synthesis of 9-bromo-5-(4-methylpiperazin-1-yl)-1H-[1,2,4]triazino[4,3-a]quinoxalin-2(3H)-one

(Z)-N″-(7-bromo-3-chloroquinoxalin-2(1H)-ylidene)-2-chloroacetohydrazide (87.0 mg, 0.249 mmol) was dissolved in 1,4-dioxane (2.49 mL), and DBU (0.0187 mL, 0.124 mmol) was added thereto. The reaction mixture was stirred at 50° C. for 1.5 hours and cooled to room temperature. After addition of 1-methylpiperazine (0.277 mL, 2.49 mmol), the reaction mixture was at room temperature for 30 minutes. The reaction mixture was concentrated under reduced pressure. The residue was purified by column chromatography (H₂O containing 0.1% formic acid:CH₃CN=60:40) on reverse-phase silica, column chromatography (DCM:MeOH=90:10) on silica, column chromatography (DCM:MeOH=97:3) on amine silica and column chromatography (H₂O containing 0.1% formic acid:CH₃CN=60:40) on reverse-phase silica, and the fractions containing the product were collected and evaporated to obtain the solid compound, 9-bromo-5-(4-methylpiperazin-1-yl)-1H-[1,2,4]triazino[4,3-a]quinoxalin-2(3H)-one (1.10 mg, 1.1%) in white.

LC/MS ESI (+): 377 (M+1)

¹H NMR (400 MHz, DMSO-d₆) δ=10.96; (s, 1H), 7.23; (s, 1H), 7.16-7.08; (m, 2H), 4.39; (s, 2H), 3.68-3.65; (m, 4H), 2.34-2.32; (m, 4H), 2.13; (s, 3H)

### Example 15: Synthesis of 8,9-dibromo-4-(4-methylpiperazin-1-yl)imidazo[1,2-a]quinoxalin-2(1H)-one

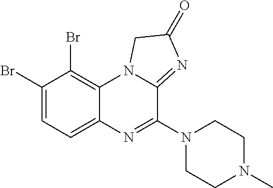 [SMILES: CN1CCN(C2=Nc3ccc(Br)c(Br)c3N3CC(=O)N=C23)CC1]

The title compound was obtained in the purification procedure of Example 12.

LC/MS ESI (+): 440 (M+1)

¹H NMR (400 MHz, CDCl₃) δ=7.70; (d, J=8.8 Hz, 1H), 7.54; (d, J=8.9 Hz, 1H), 5.25; (s, 2H), 3.87-3.74; (m, 4H), 2.58; (t, J=4.7 Hz, 4H), 2.36; (s, 3H)

### Example 16: Synthesis of N-(4-(3-(methylamino)azetidin-1-yl)-2-oxo-1,2-dihydroimidazo[1,2-a]quinoxalin-8-yl)methanesulfonamide

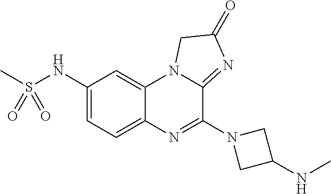

(a) Synthesis of tert-butyl methyl(1-(8-(methylsulfonamido)-2-oxo-1,2-dihydroimidazo[1,2-a]quinoxalin-4-yl)azetidin-3-yl)carbamate

Tert-butyl (1-(8-amino-2-oxo-1,2-dihydroimidazo[1,2-a]quinoxalin-4-yl)azetidin-3-yl)(methyl)carbamate (100 mg, 0.260 mmol) was dissolved in pyridine (867 mL), and methanesulfonyl chloride (26.4 mL, 0.338 mmol) was added thereto. The reaction mixture was stirred at room temperature for 1 hour. After addition of H₂O (50.0 mL), the reaction mixture was extracted with EtOAc (50.0 mL). The organic layer was washed with brine, dried over anhydrous Na₂SO₄, filtered and distilled under reduced pressure. The residue was purified by column chromatography (n-Hex:EtOAc=1:1) on silica, and the fractions containing the product were collected and evaporated to obtain the solid compound, tert-butyl methyl(1-(8-(methylsulfonamido)-2-oxo-1,2-dihydroimidazo[1,2-a]quinoxalin-4-yl)azetidin-3-yl)carbamate (86.0 mg, 71%) in yellow.

LC/MS ESI (+): 463 (M+1)

¹H NMR (400 MHz, CDCl₃) δ=7.66; (d, J=8.8 Hz, 1H), 7.57; (s, 1H), 7.34-7.28; (m, 1H), 6.50; (s, 1H), 5.14; (s, 2H), 5.09-4.71; (m, 1H), 4.55; (m, 2H), 4.35; (dd, J=6.3, 9.7 Hz, 2H), 3.05; (s, 3H), 2.98; (s, 3H), 1.48; (s, 9H)

(b) Synthesis of N-(4-(3-(methylamino)azetidin-1-yl)-2-oxo-1,2-dihydroimidazo[1,2-a]quinoxalin-8-yl)methanesulfonamide

Tert-butyl methyl(1-(8-(methylsulfonamido)-2-oxo-1,2-dihydroimidazo[1,2-a]quinoxalin-4-yl)azetidin-3-yl)carbamate (86.0 mg, 0.186 mmol) was dissolved in DCM (1.86 mL), and TFA (430 mL, 5.58 mmol) was added thereto. The reaction mixture was stirred at room temperature for 1.5 hours. After addition of saturated NaHCO₃ aqueous solution, the reaction mixture was extracted with DCM (50.0 mL). The organic layer was washed with brine, dried over anhydrous Na₂SO₄, filtered and distilled under reduced pressure. The residue was purified by column chromatography (DCM:MeOH=9:1) on silica, and the fractions containing the product were collected and evaporated to obtain the solid compound, N-(4-(3-(methylamino)azetidin-1-yl)-2-oxo-1,2-dihydroimidazo[1,2-a]quinoxalin-8-yl)methanesulfonamide (44.0 mg, 65%) in ivory.

LC/MS ESI (+): 363 (M+1)

¹H NMR (400 MHz, DMSO-d₆) δ=7.58; (d, J=8.8 Hz, 1H), 7.48; (d, J=2.4 Hz, 1H), 7.34; (dd, J=2.5, 8.9 Hz, 1H), 5.36; (s, 2H), 4.36; (m, 2H), 3.93 (m, 2H), 3.57; (s, 1H), 3.00; (s, 3H), 2.25; (s, 3H)

### Example 17: Synthesis of 8-chloro-4-(4-methylpiperazin-1-yl)imidazo[1,2-a]pyrido[2,3-e]pyrazin-2(1H)-one

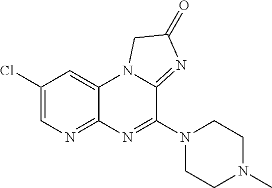 [SMILES: CN1CCN(C2=Nc3ncc(Cl)cc3N3CC(=O)N=C23)CC1]

(a) Synthesis of 2,7-dichloro-3-(4-methylpiperazin-1-yl)pyrido[2,3-b]pyrazine

2,3,7-Trichloropyrido[2,3-b]pyrazine (200 mg, 0.853 mmol) was dissolved in DCM (4.26 mL), and TEA (238 mL, 1.70 mmol) and 1-methylpiperazine (104 mL, 0.938 mmol) were added thereto at room temperature. The reaction mixture was stirred at room temperature for 2 hours. The reaction mixture was purified by column chromatography (n-Hex:EtOAc=1:1) on amine silica, and the fractions containing the product were collected and evaporated to obtain the solid compound, 2,7-dichloro-3-(4-methylpiperazin-1-yl)pyrido[2,3-b]pyrazine (61.0 mg, 44%) in ivory.

LC/MS ESI (+): 298 (M+1)

¹H NMR (400 MHz, CDCl₃) δ=8.88; (s, 1H), 8.19; (s, 1H), 3.82; (brs, 4H), 2.69-2.62; (m, 4H), 2.40; (s, 3H)

(b) Synthesis of N-(7-chloro-3-(4-methylpiperazin-1-yl)pyrido[2,3-b]pyrazin-2-yl)-2-hydroxyacetamide

2,7-Dichloro-3-(4-methylpiperazin-1-yl)pyrido[2,3-b]pyrazine (61.0 mg, 0.250 mmol) was dissolved in DMF (2.05 mL), and glycolamide (23.0 mg, 0.307 mmol) and anhydrous K₂CO₃ (42.4 mg, 0.307 mmol) were added thereto at room temperature. The reaction mixture was stirred at 70° C. for 1.5 hours. The reaction mixture was purified by column chromatography (H₂O containing 0.1% formic acid:CH₃CN=80:20) on reverse-phase silica, and the fractions containing the product were collected and evaporated to obtain the solid compound, N-(7-chloro-3-(4-methylpiperazin-1-yl)pyrido[2,3-b]pyrazin-2-yl)-2-hydroxyacetamide (55.0 mg, 70%) in ivory.

LC/MS ESI (+): 337 (M+1)

¹H NMR (400 MHz, DMSO-d₆) δ=8.63; (d, J=2.6 Hz, 1H), 8.08; (d, J=2.4 Hz, 1H), 7.59; (brs, 1H), 7.29; (brs, 1H), 4.94; (s, 2H), 3.87; (brs, 4H), 3.39-3.33; (m, 4H), 2.24; (s, 3H)

(c) Synthesis of 8-chloro-4-(4-methylpiperazin-1-yl)imidazo[1,2-a]pyrido[2,3-e]pyrazin-2(1H)-one

N-(7-chloro-3-(4-methylpiperazin-1-yl)pyrido[2,3-b]pyrazin-2-yl)-2-hydroxyacetamide (55.0 mg, 0.163 mmol) was dissolved in DMF (1.63 mL), and methanesulfonyl chloride (191 mL, 2.45 mmol) and TEA (341 mL, 2.45 mmol) were added thereto at room temperature. The reaction mixture was stirred at 70° C. for 6.5 hours. After addition of saturated NaHCO₃ aqueous solution, the reaction mixture was extracted with EtOAc (50.0 mL). The organic layer was washed with H₂O and brine, dried over anhydrous Na₂SO₄, filtered and distilled under reduced pressure. The residue was purified by column chromatography (DCM:MeOH=9:1) on silica, and the fractions containing the product were collected and evaporated to obtain the solid compound, 8-chloro-4-(4-methylpiperazin-1-yl)imidazo[1,2-a]pyrido[2,3-e]pyrazin-2(1H)-one (9.30 mg, 13%) in ivory.

LC/MS ESI (+): 319 (M+1)

¹H NMR (400 MHz, CDCl₃) δ=8.73; (d, J=2.4 Hz, 1H), 8.07; (d, J=2.6 Hz, 1H), 5.21; (s, 2H), 4.00-3.92; (m, 4H), 2.64-2.57; (m, 4H), 2.38; (s, 3H)

### Example 18: Synthesis of 8-amino-4-(3-(methylamino)azetidin-1-yl)imidazo[1,2-a]quinoxalin-2(1H)-one

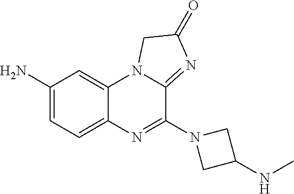 [SMILES: CNC1CN(C2=Nc3ccc(N)cc3N3CC(=O)N=C23)C1]

Tert-butyl (1-(8-amino-2-oxo-1,2-dihydroimidazo[1,2-a]quinoxalin-4-yl)azetidin-3-yl)(methyl)carbamate (100 mg, 0.260 mmol) was dissolved in DCM (2.60 mL), and TFA (100 mL, 1.30 mmol) was added thereto. The reaction mixture was stirred at room temperature for 1 hour. After addition of saturated NaHCO 3 aqueous solution, the reaction mixture was extracted with DCM (50.0 mL). The organic layer was washed with brine, dried over anhydrous Na₂SO₄, filtered and distilled under reduced pressure. The residue was purified by column chromatography (DCM:MeOH=9:1) on silica, and the fractions containing the product were collected and evaporated to obtain the solid compound, 8-amino-4-(3-(methylamino)azetidin-1-yl)imidazo[1,2-a]quinoxalin-2(1H)-one (40.0 mg, 54%) in yellow.

LC/MS ESI (+): 285 (M+1)

¹H NMR (400 MHz, DMSO-d₆) δ=7.34; (d, J=8.8 Hz, 1H), 6.86; (dd, J=2.5, 8.7 Hz, 1H), 6.74; (d, J=2.6 Hz, 1H), 5.33; (s, 2H), 5.29; (s, 2H), 4.27-4.20; (m, 2H), 3.82; (dd, J=5.6, 9.0 Hz, 2H), 3.54; (m, 1H), 2.24; (s, 3H)

### Example 19: Synthesis of 8-chloro-4-(3-(methylamino)azetidin-1-yl)imidazo[1,2-a]quinoxalin-2(1H)-one

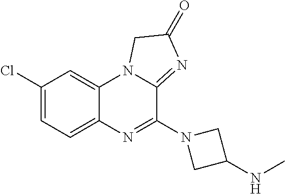 [SMILES: CNC1CN(C2=Nc3ccc(Cl)cc3N3CC(=O)N=C23)C1]

(a) Synthesis of tert-butyl (1-(8-chloro-2-oxo-1,2-dihydroimidazo[1,2-a]quinoxalin-4-yl)azetidin-3-yl)(methyl)carbamate

Tert-butyl (1-(8-amino-2-oxo-1,2-dihydroimidazo[1,2-a]quinoxalin-4-yl)azetidin-3-yl)(methyl)carbamate (300 mg, 0.780 mmol) was dissolved in CH₃CN (7.80 mL), and tert-butyl nitrite (124 mL, 0.936 mmol), p-toluene sulfonic acid (178 mg, 0.936 mmol), CuCl₂(10.5 mg, 0.0780 mmol) and TBAC (260 mg, 0.936 mmol) were added thereto at room temperature. The reaction mixture was stirred at 40° C. for 22 hours. The reaction mixture was purified by column chromatography (H₂O containing 0.1% formic acid:CH₃CN=65:35) on reverse-phase silica, and the fractions containing the product were collected and distilled under reduced pressure. The residue was purified by column chromatography (n-Hex:EtOAc=3:1) on silica, and the fractions containing the product were collected and evaporated to obtain the solid compound, tert-butyl (1-(8-chloro-2-oxo-1,2-dihydroimidazo[1,2-a]quinoxalin-4-yl)azetidin-3-yl)(methyl)carbamate (60.0 mg, 19%) in white.

LC/MS ESI (+): 404 (M+1)

¹H NMR (400 MHz, CDCl₃) δ=7.71; (d, J=2.3 Hz, 1H), 7.62; (d, J=8.8 Hz, 1H), 7.44; (dd, J=2.4, 8.8 Hz, 1H), 5.15; (s, 2H), 5.11 4.81; (m, 1H), 4.57; (m, 2H), 4.38; (dd, J=6.0, 10.1 Hz, 2H), 3.00; (s, 3H), 1.50; (s, 9H)

(b) Synthesis of 8-chloro-4-(3-(methylamino)azetidin-1-yl)imidazo[1,2-a]quinoxalin-2(1H)-one

Tert-butyl (1-(8-chloro-2-oxo-1,2-dihydroimidazo[1,2-a]quinoxalin-4-yl)azetidin-3-yl)(methyl)carbamate (60.0 mg, 0.194 mml) was dissolved in DCM (1.48 mL), and TFA (114 mL, 1.48 mmol) was added thereto. The reaction mixture was stirred at room temperature for 18 hours. After addition of saturated NaHCO₃ aqueous solution, the reaction mixture was extracted with DCM (50.0 mL). The organic layer was washed with brine, dried over anhydrous Na₂SO₄, filtered and distilled under reduced pressure. The residue was purified by column chromatography (DCM:MeOH=9:1) on silica, and the fractions containing the product were collected and evaporated to obtain the solid compound, 8-chloro-4-(3-(methylamino)azetidin-1-yl)imidazo[1,2-a]quinoxalin-2(1H)-one (36.0 mg, 80%) in white.

LC/MS ESI (+): 304 (M+1)

¹H NMR (400 MHz, CDCl₃) δ=7.69; (d, J=2.4 Hz, 1H), 7.60; (d, J=8.8 Hz, 1H), 7.42; (dd, J=2.3, 8.8 Hz, 1H), 5.15; (s, 2H), 4.55; (dd, J=7.5, 9.3 Hz, 2H), 4.08; (dd, J=4.8, 9.8 Hz, 2H), 3.79-3.72; (m, 1H), 2.49; (s, 3H)

### Example 20: Synthesis of 8-chloro-4-(3-(methylamino)azetidin-1-yl)imidazo[1,2-a]pyrido[2,3-e]pyrazin-2(1H)-one

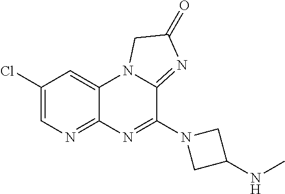 [SMILES: CNC1CN(C2=Nc3ncc(Cl)cc3N3CC(=O)N=C23)C1]

(a) Synthesis of tert-butyl (1-(2,7-dichloropyrido[2,3-b]pyrazin-3-yl)azetidin-3-yl)(methyl)carbamate

2,3,7-Trichloropyrido[2,3-b]pyrazine (267 mg, 1.13 mmol) was dissolved in DCM (11.4 mL), and TEA (0.952 mL, 6.83 mmol) and tert-butyl azetidin-3-yl-(methyl)carbamate hydrochloride (279 mg, 1.25 mmol) were added thereto. The reaction mixture was stirred at room temperature for 1 hour. The reaction mixture was purified by column chromatography (n-Hex:EtOAc=1:1) on silica, and the fractions containing the product were collected and evaporated to obtain the solid compound, tert-butyl (1-(2,7-dichloropyrido[2,3-b]pyrazin-3-yl)azetidin-3-yl)(methyl)carbamate (94.0 mg, 21%) in ivory.

LC/MS ESI (+): 384 (M+1)

¹H NMR (400 MHz, CDCl₃) δ=8.80; (s, 1H), 8.12; (s, 1H), 5.22-4.90; (m, 1H), 4.90-4.70; (m, 2H), 4.65-4.50; (m, 2H), 3.00; (s, 3H), 1.50; (s, 9H)

(b) Synthesis of tert-butyl (1-(7-chloro-2-(2-hydroxyacetamido)pyrido[2,3-b]pyrazin-3-yl)azetidin-3-yl)(methyl)carbamate

Tert-butyl (1-(2,7-dichloropyrido[2,3-b]pyrazin-3-yl)azetidin-3-yl)(methyl)carbamate (94.0 mg, 0.245 mmol) was dissolved in DMF (2.44 mL), and glycolamide (27.5 mg, 0.367 mmol) and anhydrous K₂CO₃ (50.7 mg, 0.367 mmol) were added thereto at room temperature. The reaction mixture was stirred at 70° C. for 1 hour. The reaction mixture was purified by column chromatography (H₂O containing 0.1% formic acid:CH₃CN=45:55) on reverse-phase silica, and the fractions containing the product were collected and evaporated to obtain the solid compound, tert-butyl (1-(7-chloro-2-(2-hydroxyacetamido)pyrido[2,3-b]pyrazin-3-yl)azetidin-3-yl)(methyl)carbamate (66.0 mg, 63%) in ivory.

LC/MS ESI (+): 423 (M+1)

¹H NMR (400 MHz, DMSO-d₆) δ=8.56; (d, J=2.6 Hz, 1H), 8.03; (d, J=2.4 Hz, 1H), 7.55; (s, 1H), 7.35; (s, 1H), 5.09-4.88; (m, 1H), 4.86; (s, 2H), 4.72-4.17; (m, 4H), 2.91; (s, 3H), 1.42; (s, 9H)

(c) Synthesis of tert-butyl (1-(8-chloro-2-oxo-1,2-dihydroimidazo[1,2-a]pyrido[2,3-e]pyrazin-4-yl)azetidin-3-yl)(methyl)carbamate

Tert-butyl (1-(7-chloro-2-(2-hydroxyacetamido)pyrido[2,3-b]pyrazin-3-yl)azetidin-3-yl)(methyl)carbamate (66.0 mg, 0.156 mmol) was dissolved in DMF (1.56 mL), and methanesulfonyl chloride (182 mL, 2.34 mmol) and TEA (326 mL, 2.34 mmol) were added thereto at room temperature. The reaction mixture was stirred at 70° C. for 3.5 hours. After addition of saturated NaHCO₃ aqueous solution, the reaction mixture was extracted with EtOAc (50.0 mL). The organic layer was washed with H₂O and brine, dried over anhydrous Na₂SO₄, filtered and distilled under reduced pressure. The residue was purified by column chromatography (n-Hex:EtOAc=1:1) on silica, and the fractions containing the product were collected and evaporated to obtain the solid compound, tert-butyl (1-(8-chloro-2-oxo-1,2-dihydroimidazo[1,2-a]pyrido[2,3-e]pyrazin-4-yl)azetidin-3-yl)(methyl)carbamate (11.0 mg, 17%) in white.

LC/MS ESI (+): 405 (M+1)

¹H NMR (400 MHz, CDCl₃) δ=8.67; (d, J=2.4 Hz, 1H), 8.01; (d, J=2.4 Hz, 1H), 5.16; (s, 2H), 4.68; (brs, 2H), 4.51; (brs, 2H), 4.17-4.02; (m, 1H), 2.99; (s, 3H), 1.50; (s, 9H)

(d) Synthesis of 8-chloro-4-(3-(methylamino)azetidin-1-yl)imidazo[1,2-a]pyrido[2,3-e]pyrazin-2(1H)-one

Tert-butyl (1-(8-chloro-2-oxo-1,2-dihydroimidazo[1,2-a]pyrido[2,3-e]pyrazin-4-yl)azetidin-3-yl)(methyl)carbamate (11.0 mg, 0.0270 mmol) was dissolved in DCM (272 mL), and TFA (10.4 mL, 0.136 mmol) was added thereto. The reaction mixture was stirred at room temperature for 2.5 hours. After addition of saturated NaHCO₃ aqueous solution, the reaction mixture was extracted with DCM (50.0 mL). The organic layer was washed with brine, dried over anhydrous Na₂SO₄, filtered and distilled under reduced pressure. The residue was purified by column chromatography (DCM:MeOH=9:1) on silica, and the fractions containing the product were collected and evaporated to obtain the solid compound, 8-chloro-4-(3-(methylamino)azetidin-1-yl)imidazo[1,2-a]pyrido[2,3-e]pyrazin-2(1H)-one (5.90 mg, 70%) in white.

LC/MS ESI (+): 305 (M+1)

¹H NMR (400 MHz, CDCl₃) δ=8.65; (d, J=2.6 Hz, 1H), 7.99; (d, J=2.4 Hz, 1H), 5.16; (s, 2H), 4.65; (brs, 2H), 4.20; (brs, 2H), 3.81-3.75; (m, 1H), 2.49; (s, 3H)

### Example 21: Synthesis of 8-bromo-4-(3-(methylamino)azetidin-1-yl)imidazo[1,2-a]quinoxalin-2(1H)-one hydrochloride

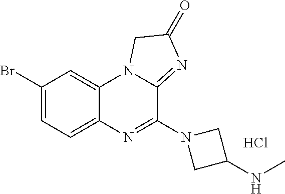 [SMILES: CNC1CN(C2=Nc3ccc(Br)cc3N3CC(=O)N=C23)C1 ; Cl]

8-Bromo-4-(3-(methylamino)azetidin-1-yl)imidazo[1,2-a]quinoxalin-2(1H)-one (10.0 mg, 0.0290 mmol) was dissolved in CH₃CN (0.500 mL) and H₂O (0.500 mL), and 1N HCl (0.0290 mL, 0.0290 mmol) was added thereto. The reaction mixture was stirred at room temperature for 1 hour and freeze-dried to obtain the solid compound, 8-bromo-4-(3-(methylamino)azetidin-1-yl)imidazo[1,2-a]quinoxalin-2(1H)-one hydrochloride (11.0 mg, 100%) in ivory.

LC/MS ESI (+): 348 (M+1)

¹H NMR (400 MHz, DMSO-d₆) δ=9.48; (brs, 2H), 7.85; (d, J=2.2 Hz, 1H), 7.67-7.63; (m, 1H), 7.59-7.56; (m, 1H), 5.39; (s, 2H), 4.59-4.49; (m, 2H), 4.40-4.35; (m, 2H), 4.16-4.09; (m, 1H), 2.60; (brt, J=5.3 Hz, 3H)

### Example 22: Synthesis of 3-chloro-6-(4-methylpiperazin-1-yl)imidazo[1,2-a]pyrido[4,3-e]pyrazin-8(9H)-one

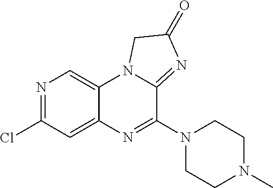 [SMILES: CN1CCN(C2=Nc3cc(Cl)ncc3N3CC(=O)N=C23)CC1]

(a) Synthesis of 7-chloro-1,4-dihydropyrido[3,4-b]pyrazin-2,3-dione

The suspension of 6-chloropyridin-3,4-diamine (1.00 g, 6.97 mmol) and diethyl oxalate (30.0 mL, 219 mmol) was stirred at 120° C. for 18 hours. The reaction mixture was cooled to room temperature, and the obtained solid was filtered, washed with EtOH and n-Hex, and dried under reduced pressure dried to obtain the solid compound, 7-chloro-1,4-dihydropyrido[3,4-b]pyrazin-2,3-dione (1.22 g, 89%) in brown.

LC/MS ESI (+): 198 (M+1)

¹H NMR (400 MHz, DMSO-d₆) δ=12.26; (s, 1H), 12.13; (s, 1H), 8.08; (s, 1H), 7.05; (s, 1H)

(b) Synthesis of 3,7-dichloro-2-(4-methylpiperazin-1-yl)pyrido[3,4-b]pyrazine

To the suspension of 7-chloro-1,4-dihydropyrido[3,4-b]pyrazin-2,3-dione (200 mg, 1.01 mmol) and SOCK (2.95 mL, 40.5 mmol) DMF (7.84 μL, 0.101 mmol) was added, and this mixture was stirred at 100° C. for 5 hours. The reaction mixture was cooled to room temperature and concentrated to 2,3,7-trichloropyrido[3,4-b]pyrazine. Obtained 2,3,7-trichloropyrido[3,4-b]pyrazine was dissolved in DCM (10.1 mL), and 1-methylpiperazine (282 μL, 2.53 mmol) was added thereto. The reaction mixture was stirred at room temperature for 30 minutes. The reaction mixture was purified by column chromatography (DCM:MeOH=20:1) on silica, and the fractions containing the product were collected and evaporated to obtain the solid compound, 3,7-dichloro-2-(4-methylpiperazin-1-yl)pyrido[3,4-b]pyrazine (220 mg, 73%) in light yellow.

LC/MS ESI (+): 298 (M+1)

¹H NMR (400 MHz, DMSO-d₆) δ=8.98; (s, 1H), 7.76; (s, 1H), 3.70; (brd, J=5.0 Hz, 4H), 3.37-3.36; (m, 4H), 2.24; (s, 3H)

(C) Synthesis of N-(7-chloro-2-(4-methylpiperazin-1-yl)pyrido[3,4-b]pyrazin-3-yl)-2-hydroxyacetamide

3,7-Dichloro-2-(4-methylpiperazin-1-yl)pyrido[3,4-b]pyrazine (220 mg, 0.738 mmol) was dissolved in DMF (3.69 mL), and glycolamide (66.5 mg, 0.885 mmol) and K₂CO₃ (153 mg, 1.11 mmol) were added thereto at room temperature. The reaction mixture was stirred at 70° C. for 1 hour. The reaction mixture was cooled to room temperature, and the obtained solid was filtered, washed with H₂O and Et₂O, and dried under reduced pressure dried to obtain the solid compound, N-(7-chloro-2-(4-methylpiperazin-1-yl)pyrido[3,4-b]pyrazin-3-yl)-2-hydroxyacetamide (175 mg, 71%) in brown.

LC/MS ESI (+): 337 (M+1)

¹H NMR (400 MHz, DMSO-d₆) δ=8.59; (s, 1H), 7.61; (brs, 1H), 7.53; (s, 1H), 7.31; (brs, 1H), 4.93; (s, 2H), 4.01-3.90; (m, 4H), 2.49-2.44; (m, 4H), 2.22; (s, 3H)

(d) Synthesis of 3-chloro-6-(4-methylpiperazin-1-yl)imidazo[1,2-a]pyrido[4,3-e]pyrazin-8(9H)-one

N-(7-chloro-2-(4-methylpiperazin-1-yl)pyrido[3,4-b]pyrazin-3-yl)-2-hydroxyacetamide (175 mg, 0.520 mmol) was dissolved in DMF (3.46 mL), and methanesulfonyl chloride (607 μL, 7.79 mmol) and TEA (1.09 mL, 7.79 mmol) were added there to at room temperature. The reaction mixture was stirred at 80° C. for 1 hour. The reaction mixture was cooled to room temperature, and H₂O and EtOAc were added thereto, and this mixture was extracted with EtOAc. The organic layer was washed with brine, dried over anhydrous Na₂SO₄, filtered and distilled under reduced pressure. The residue was purified by column chromatography (DCM:MeOH=30:1) on silica, and the fractions containing the product were collected and evaporated to obtain the solid compound, 3-chloro-6-(4-methylpiperazin-1-yl)imidazo[1,2-a]pyrido[4,3-e]pyrazin-8(9H)-one (115 mg, 69%) in light yellow.

LC/MS ESI (+): 319 (M+1)

¹H NMR (400 MHz, CDCl₃) δ=8.79; (s, 1H), 7.54; (s, 1H), 5.20; (s, 2H), 4.04-3.89; (m, 4H), 2.59; (brs, 4H), 2.38; (s, 3H)

### Example 23: Synthesis of 8-chloro-4-(3-(methylamino)azetidin-1-yl)imidazo[1.2-a]pyrido[3.4-e]pyrazin-2(1H)-one hydrochloride

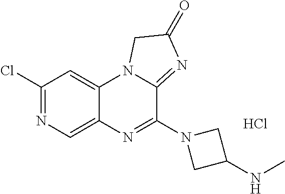 [SMILES: CNC1CN(C2=Nc3cnc(Cl)cc3N3CC(=O)N=C23)C1 ; Cl]

(a) Synthesis of N-(3,7-dichloropyrido[3,4-b]pyrazin-2-yl)-2-hydroxyacetamide DMF (0.0780 mL, 1.01 mmol) was added to the suspension of 7-chloro-1,4-dihydropyrido[3,4-b]pyrazin-2,3-dione (1.00 g, 5.06 mmol) and SOCl₂ (12.9 mL, 177 mmol) at room temperature, and this mixture was stirred at 100° C. for 5 hours. The reaction mixture was cooled to room temperature and concentrated to 2,3,7-trichloropyrido[3,4-b]pyrazine. Obtained 2,3,7-trichloropyrido[3,4-b]pyrazine and glycolamide (760 mg, 10.1 mmol) were dissolved in DMF (16.9 mL), and DIPEA (1.76 mL, 10.1 mmol) was added thereto. The reaction mixture was stirred at 80° C. for 1 hour. The reaction mixture was cooled to room temperature, H₂O and EtOAc were added thereto, and this mixture was extracted with EtOAc. The organic layer was washed with brine, dried over anhydrous Na₂SO₄, filtered and distilled under reduced pressure. The residue was purified by column chromatography (n-Hex:EtOAc=1:1) on silica, and the fractions containing the product were collected and evaporated to obtain the solid compound, N-(3,7-dichloropyrido[3,4-b]pyrazin-2-yl)-2-hydroxyacetamide (140 mg, 10%) in light yellow.

LC/MS ESI (+): 273 (M+1)

¹H NMR (400 MHz, DMSO-d₆) δ=9.16; (s, 1H), 7.94; (s, 1H), 7.60; (brs, 1H), 7.41; (brs, 1H), 5.01; (s, 2H)

(b) Synthesis of tert-butyl (1-(7-chloro-2-(2-hydroxyacetamido)pyrido[3,4-b]pyrazin-3-yl)azetidin-3-yl)(methyl)carbamate

N-(3,7-dichloropyrido[3,4-b]pyrazin-2-yl)-2-hydroxyacetamide (140 mg, 0.513 mmol) and tert-butyl azetidin-3-yl(methyl)carbamate hydrochloride (171 mg, 0.769 mmol) were dissolved in DMF (2.56 mL), and TEA (286 μL, 2.05 mmol) was added thereto at room temperature. The reaction mixture was stirred at room temperature for 30 minutes and purified by column chromatography (H₂O containing 0.1% formic acid:CH₃CN=95:5˜0:100) on reverse-phase silica, and the fractions containing the product were collected and evaporated to obtain the solid compound, tert-butyl (1-(7-chloro-2-(2-hydroxyacetamido)pyrido[3,4-b]pyrazin-3-yl)azetidin-3-yl)(methyl)carbamate (150 mg, 69%) in light brown.

LC/MS ESI (+): 423 (M+1)

¹H NMR (400 MHz, DMSO-d₆) δ=8.65; (s, 1H), 7.57; (s, 1H), 7.55; (s, 1H), 7.37; (s, 1H), 4.89; (m, 3H), 4.67-4.14; (m, 4H), 2.90; (s, 3H), 1.41; (s, 9H)

(c) Synthesis of tert-butyl (1-(8-chloro-2-oxo-1,2-dihydroimidazo[1,2-a]pyrido[3,4-e]pyrazin-4-yl)azetidin-3-yl)(methyl)carbamate

Tert-butyl (1-(7-chloro-2-(2-hydroxyacetamido)pyrido[3,4-b]pyrazin-3-yl)azetidin-3-yl)(methyl)carbamate (140 mg, 0.331 mmol) and methane-sulfonyl chloride (387 μL, 4.97 mmol) were dissolved in DMF (2.21 mL), and pyridine (26.8 μL) was added thereto at room temperature. The reaction mixture was stirred at 80° C. for 1 hour. The reaction mixture was cooled to room temperature and purified by column chromatography (H₂O containing 0.1% formic acid:CH₃CN=95:5˜0:100) on reverse-phase silica, and the fractions containing the product were collected and evaporated to obtain the solid compound, tert-butyl (1-(8-chloro-2-oxo-1,2-dihydroimidazo[1,2-a]pyrido[3,4-e]pyrazin-4-yl)azetidin-3-yl)(methyl)carbamate (72.0 mg, 54%) in white.

LC/MS ESI (+): 405 (M+1)

¹H NMR (400 MHz, CDCl₃) δ=8.81; (s, 1H), 7.58; (s, 1H), 5.17; (s, 2H), 5.12-4.91; (m, 1H), 4.60; (t, J=8.3 Hz, 2H), 4.51-4.36 (m, 2H), 2.99; (s, 3H), 1.49; (s, 9H)

(d) Synthesis of 8-chloro-4-(3-(methylamino)azetidin-1-yl)imidazo[1,2-a]pyrido[3,4-e]pyrazin-2(1H)-one hydrochloride

Tert-butyl (1-(8-chloro-2-oxo-1,2-dihydroimidazo[1,2-a]pyrido[3,4-e]pyrazin-4-yl)azetidin-3-yl)(methyl)carbamate (70.0 mg, 0.173 mmol) was dissolved in DCM (1.73 mL), and TFA (265 μL, 3.46 mmol) was added thereto. The reaction mixture was stirred at room temperature for 40 minutes. After addition of 1N HCl, the reaction mixture was washed with EtOAc, neutralized by 1N NaOH and extracted with EtOAc. The organic layer was washed with brine, dried over anhydrous Na₂SO₄, filtered and distilled under reduced pressure. The residue was purified by column chromatography (DCM:MeOH=20:1) on silica, and the fractions containing the product were collected and evaporated to obtain the solid compound, 8-chloro-4-(3-(methylamino)azetidin-1-yl)imidazo[1,2-a]pyrido[3,4-e]pyrazin-2(1H)-one hydrochloride (13.0 mg, 22%) in white.

LC/MS ESI (+): 305 (M+1)

¹H NMR (400 MHz, DMSO-d₆) δ=9.26; (brs, 2H), 8.76; (s, 1H), 7.73 (s, 1H), 5.44; (s, 2H), 4.71-4.49; (m, 2H), 4.47-4.27; (m, 2H), 4.21-4.08; (m, 1H), 2.65-2.61; (m, 3H)

### Example 24: Synthesis of 8-chloro-4-(3-(methylamino)azetidin-1-yl)imidazo[1,2-a]pyrido[3,4-e]pyrazin-2(1H)-one

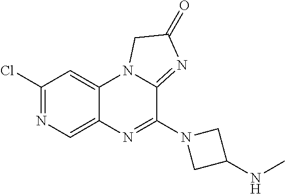 [SMILES: CNC1CN(C2=Nc3cnc(Cl)cc3N3CC(=O)N=C23)C1]

Tert-butyl (1-(8-chloro-2-oxo-1,2-dihydroimidazo[1,2-a]pyrido[3,4-e]pyrazin-4-yl)azetidin-3-yl)(methyl)carbamate (30.0 mg, 0.0740 mmol) was dissolved in DCM (741 μL), and TFA (113 μL, 1.48 mmol) was added thereto. The reaction mixture was stirred at room temperature for 30 minutes. The reaction mixture was purified by column chromatography (DCM:MeOH=20:1) on amine silica, and the fractions containing the product were collected and evaporated to obtain the solid compound, 8-chloro-4-(3-(methylamino)azetidin-1-yl)imidazo[1,2-a]pyrido[3,4-e]pyrazin-2(1H)-one (11.0 mg, 49%) in white.

LC/MS ESI (+): 305 (M+1)

¹H NMR (400 MHz, DMSO-d₆) δ=8.68; (s, 1H), 7.64; (s, 1H), 5.39; (s, 2H), 4.63-4.30; (m, 2H), 4.17-3.87; (m, 2H), 3.58; (m, 1H), 2.25; (s, 3H)

### Example 25: Synthesis of 8-bromo-4-(4-methylpiperazin-1-yl)imidazo[1,2-a]pyrido[3,4-e]pyrazin-2(1H)-one

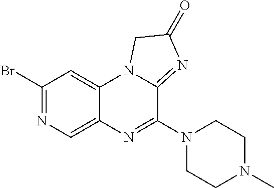 [SMILES: CN1CCN(C2=Nc3cnc(Br)cc3N3CC(=O)N=C23)CC1]

(a) Synthesis of 7-bromo-1,4-dihydropyrido[3,4-b]pyrazin-2,3-dione

The suspension of 6-bromopyridin-3,4-diamine (1.00 g, 5.32 mmol) and diethyl oxalate (22.9 mL, 168 mmol) was stirred at 120° C. for 14 hours. The reaction mixture was cooled to room temperature. The obtained solid was filtered, washed with EtOH and n-Hex, and dried under reduced pressure to obtain the solid compound, 7-bromo-1,4-dihydropyrido[3,4-b]pyrazin-2,3-dione (1.10 g, 85%) in brown.

LC/MS ESI (+): 242 (M+1)

¹H NMR (400 MHz, DMSO-d₆) δ=12.16; (brs, 2H), 8.07; (s, 1H), 7.17; (s, 1H)

(b) Synthesis of N-(7-bromo-3-chloropyrido[3,4-b]pyrazin-2-yl)-2-hydroxyacetamide

DMF (77.0 μL, 0.992 mmol) was added to the suspension of 7-bromo-1,4-dihydropyrido[3,4-b]pyrazin-2,3-dione (600 mg, 2.48 mmol) and SOCl₂ (7.23 mL, 99.0 mmol) at room temperature, and this mixture was stirred at 100° C. for 5 hours. The reaction mixture was cooled to room temperature and concentrated to 7-bromo-2,3-dichloropyrido[3,4-b]pyrazine. Obtained 7-bromo-2,3-dichloropyrido[3,4-b]pyrazine and glycolamide (111 mg, 1.48 mmol) were dissolved in sulfolane (8.23 mL), and DIPEA (473 μL, 2.72 mmol) was added thereto. The reaction mixture was stirred at 60° C. for 2 hours. The reaction mixture was cooled to room temperature, H₂O and EtOAc were added thereto, and this mixture was extracted with EtOAc. The organic layer was washed with brine, dried over anhydrous Na₂SO₄, filtered and distilled under reduced pressure. The residue was purified by column chromatography (n-Hex:EtOAc=1:1) on silica, and the fractions containing the product were collected and evaporated to obtain the solid compound, N-(7-bromo-3-chloropyrido[3,4-b]pyrazin-2-yl)-2-hydroxyacetamide (105 mg, 14%) in light yellow.

LC/MS ESI (+): 317 (M+1)

¹H NMR (400 MHz, DMSO-d₆) δ=9.13; (s, 1H), 8.08; (s, 1H), 7.60; (brs, 1H), 7.41; (brs, 1H), 5.01; (s, 2H)

(C) Synthesis of N-(7-bromo-3-(4-methylpiperazin-1-yl)pyrido[3,4-b]pyrazin-2-yl)-2-hydroxyacetamide

N-(7-bromo-3-chloropyrido[3,4-b]pyrazin-2-yl)-2-hydroxyacetamide (40.0 mg, 0.126 mmol) was dissolved in DMF (630 μL), and 1-methylpiperazine (28.0 μL, 0.252 mmol) was added thereto. The reaction mixture was stirred at 60° C. for 10 minutes. The reaction mixture was cooled to room temperature, H₂O and EtOAc were added thereto, and this mixture was extracted with EtOAc. The organic layer was washed with brine, dried over anhydrous Na₂SO₄, filtered and distilled under reduced pressure to obtain the solid compound, N-(7-bromo-3-(4-methylpiperazin-1-yl)pyrido[3,4-b]pyrazin-2-yl)-2-hydroxyacetamide (42.0 mg, 87%) in light brown.

LC/MS ESI (+): 381 (M+1)

¹H NMR (400 MHz, DMSO-d₆) δ=8.71-8.70; (s, 1H), 7.72-7.71; (s, 1H), 7.61 (brs, 1H), 7.35-7.29; (m, 1H), 4.98-4.96; (m, 2H), 3.81-3.78; (m, 4H), 2.48-2.46;; (m, 4H), 2.23; (s, 3H)

(d) Synthesis of 8-bromo-4-(4-methylpiperazin- 1-yl)imidazo[1,2-a]pyrido[3,4-e]pyrazin-2(1H)-one

N-(7-bromo-3-(4-methylpiperazin-1-yl)pyrido[3,4-b]pyrazin-2-yl)-2-hydroxyacetamide (42.0 mg, 0.110 mmol) was dissolved in DMF (551 μL), and methanesulfonyl chloride (129 μL, 1.65 mmol) and pyridine (267 μL, 3.31 mmol) were added thereto at room temperature. The reaction mixture was stirred at 80° C. for 1 hour. The reaction mixture was cooled to room temperature and purified by column chromatography (H₂O containing 0.1% formic acid:CH₃CN=95:5-0:100) on reverse-phase silica, and the fractions containing the product were collected and evaporated to obtain the solid compound, 8-bromo-4-(4-methylpiperazin-1-yl)imidazo[1,2-a]pyrido[3,4-e]pyrazin-2(1H)-one (2.00 mg, 5%) in brown.

LC/MS ESI (+): 363 (M+1)

¹H NMR (400 MHz, DMSO-d₆) δ=8.70; (s, 1H), 7.79; (s, 1H), 5.35; (s, 2H), 3.69-3.65; (m, 4H), 2.51-2.46; (m, 4H), 2.21 2.19; (m, 3H)

### Example 26: Synthesis of 8-bromo-4-(3-(methylamino)azetidin-1-yl)imidazo[1,2-a]pyrido[3,4-e]pyrazin-2(1H)-one

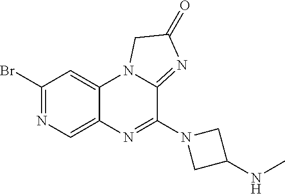 [SMILES: CNC1CN(C2=Nc3cnc(Br)cc3N3CC(=O)N=C23)C1]

(a) Synthesis of tert-butyl (1-(7-bromo-2-(2-hydroxyacetamido)pyrido[3,4-b]pyrazin-3-yl)azetidin-3-yl)(methyl)carbamate

N-(7-bromo-3-chloropyrido[3,4-b]pyrazin-2-yl)-2-hydroxyacetamide (66.0 mg, 0.208 mmol) and tert-butyl azetidin-3-yl(methyl)carbamate hydrochloride (69.4 mg, 0.312 mmol) were dissolved in DMF (1.04 mL), and TEA (116 μL, 0.831 mmol) was added thereto at room temperature. The reaction mixture was stirred at room temperature for 30 minutes and purified by column chromatography (H₂O containing 0.1% formic acid:CH₃CN=95:5-0:100) on reverse-phase silica, and the fractions containing the product were collected and evaporated to obtain the solid compound, tert-butyl (1-(7-bromo-2-(2-hydroxyacetamido)pyrido[3,4-b]pyrazin-3-yl)azetidin-3-yl)(methyl) carbamate (82.0 mg, 84%) in white.

LC/MS ESI (+): 467 (M+1)

¹H NMR (400 MHz, DMSO-d₆) δ=8.63; (s, 1H), 7.67; (s, 1H), 7.57; (brs, 1H), 7.36; (brs, 1H), 4.89; (m, 3H), 4.71-4.21; (m, 4H), 2.90; (s, 3H), 1.41; (s, 9H)

(b) Synthesis of tert-butyl (1-(8-bromo-2-oxo-1,2-dihydroimidazo[1,2-a]pyrido[3,4-e]pyrazin-4-yl)azetidin-3-yl)(methyl)carbamate

Tert-butyl (1-(7-bromo-2-(2-hydroxyacetamido)pyrido[3,4-b]pyrazin-3-yl)azetidin-3-yl)(methyl)carbamate (80.0 mg, 0.171 mmol) was dissolved in DMF (856 μL), and methanesulfonyl chloride (200 μL, 2.57 mmol) and pyridine (415 μL, 5.14 mmol) were added thereto at room temperature. The reaction mixture was stirred at 80° C. for 1 hour. The reaction mixture was cooled to room temperature and purified by column chromatography (H₂O containing 0.1% formic acid:CH₃CN=95:5-0:100) on reverse-phase silica, and the fractions containing the product were collected and evaporated to obtain the solid compound, tert-butyl (1-(8-bromo-2-oxo-1,2-dihydroimidazo[1,2-a]pyrido[3,4-e]pyrazin-4-yl)azetidin-3-yl)(methyl)carbamate (64.0 mg, 83%) in white.

LC/MS ESI (+): 449 (M+1)

¹H NMR (400 MHz, DMSO-d₆) δ=8.69; (s, 1H), 7.78; (s, 1H), 5.38; (s, 2H), 5.03-4.73; (m, 1H), 4.68-4.14; (m, 4H), 2.89; (s, 3H), 1.41; (s, 9H)

(c) Synthesis of 8-bromo-4-(3-(methylamino)azetidin-1-yl)imidazo[1,2-a]pyrido[3,4-e]pyrazin-2(1H)-one

Tert-butyl (1-(8-bromo-2-oxo-1,2-dihydroimidazo[1,2-a]pyrido[3,4-e]pyrazin-4-yl)azetidin-3-yl)(methyl)carbamate (62.0 mg, 0.138 mmol) was dissolved in DCM (920 μL), and TFA (211 μL, 2.76 mmol) was added thereto. The reaction mixture was stirred at room temperature for 30 minutes. The reaction mixture was purified by column chromatography (DCM:MeOH=20:1) on amine silica and column chromatography (DCM:MeOH=20:1) on silica, and the fractions containing the product were collected and evaporated to obtain the solid compound, 8-bromo-4-(3-(methylamino)azetidin-1-yl)imidazo[1,2-a]pyrido[3,4-e]pyrazin-2(1H)-one (32.0 mg, 66%) in white.

LC/MS ESI (+): 349 (M+1)

¹H NMR (400 MHz, DMSO-d₆) δ=8.66; (s, 1H), 7.77; (s, 1H), 5.39; (s, 2H), 4.60-4.27; (m, 2H), 4.23-3.84; (m, 2H), 3.63-3.54; (m, 1H), 2.26; (s, 3H)

### Example 27: Synthesis of 8-bromo-4-(4-methylpiperazin-1-yl)-1H-pyrido[2,3-e][1,2,4]thiadiazolo[4,3-a]pyrazin 2,2-dioxide

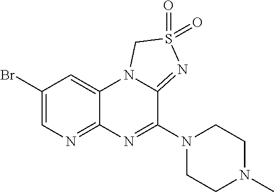 [SMILES: CN1CCN(C2=Nc3ncc(Br)cc3N3CS(=O)(=O)N=C23)CC1]

(a) Synthesis of 7-bromo-3-(4-methylpiperazin-1-yl)pyrido[2,3-b]pyrazin-2-amine

The suspension of 7-bromo-2-chloro-3-(4-methylpiperazin-1-yl)pyrido[2,3-b]pyrazine (420 mg, 1.22 mmol) and 2M NH₃ IPA solution (6.13 mL, 12.2 mmol) was stirred at 100° C. for 21 hours. n-Hex was added to the reaction mixture, and the obtained solid was filtered and dried under reduced pressure to obtain the solid compound, 7-bromo-3-(4-methylpiperazin-1-yl)pyrido[2,3-b]pyrazin-2-amine (372 mg, 94%) in white.

LC/MS ESI (+): 323 (M+1)

¹H NMR (400 MHz, CDCl₃) δ=8.70; (d, J=2.4 Hz, 1H), 8.08; (d, J=2.3 Hz, 1H), 5.13; (brs, 2H), 3.57; (brs, 4H), 2.67; (brs, 4H), 2.41; (s, 3H)

(b) Synthesis of 8-bromo-4-(4-methylpiperazin-1-yl)-1H-pyrido[2,3-e][1,2,4]thiadiazolo[4,3-a]pyrazin 2,2-dioxide

7-Bromo-3-(4-methylpiperazin-1-yl)pyrido[2,3-b]pyrazin-2-amine (270 mg, 0.835 mmol) was dissolved in pyridine (2.78 mL), and chloromethanesulfonyl chloride (1.51 mL, 16.7 mmol) was added thereto at 0° C. The reaction mixture was stirred at room temperature for 17 hours. After addition of saturated NaHCO₃ aqueous solution, the reaction mixture was extracted with EtOAc (100 mL). The organic layer was washed with brine, dried over anhydrous Na₂SO₄, filtered and distilled under reduced pressure. The residue was purified by column chromatography (DCM:MeOH=9:1) on silica, and the fractions containing the product were collected and evaporated. The residue was purified by plate TLC (EtOAc:MeOH=9:1), and the fractions containing the product were collected and evaporated. The residue was stirred in Et₂O for 30 minutes and filtered to obtain the solid compound, 8-bromo-4-(4-methylpiperazin-1-yl)-1H -pyrido[2,3-e][1,2,4]thiadiazolo[4,3-a]pyrazin 2,2-dioxide (6.50 mg, 2%) in yellow.

LC/MS ESI (+): 399 (M+1)

¹H NMR (400 MHz, CDCl₃) δ=8.56; (d, J=2.0 Hz, 1H), 7.39; (d, J=2.1 Hz, 1H), 4.86 ;(s, 2H), 4.30; (brs, 4H), 2.59; (brt, J=4.8 Hz, 4H), 2.36; (s, 3H)

### Example 28: Synthesis of 8-chloro-4-(4-methylpiperazin-1-yl)imidazo[1,2-a]pyrido[3,4-e]pyrazin-2(1H)-one hydrochloride

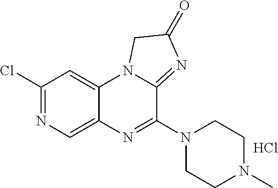 [SMILES: CN1CCN(C2=Nc3cnc(Cl)cc3N3CC(=O)N=C23)CC1 ; Cl]

(a) Synthesis of N-(7-chloro-3-(4-methylpiperazin-1-yl)pyrido[3,4-b]pyrazin-2-yl)-2-hydroxyacetamide

N-(3,7-dichloropyrido[3,4-b]pyrazin-2-yl)-2-hydroxyacetamide (190 mg, 0.696 mmol) was dissolved in DMF (2.32 mL), and 1-methylpiperazine (155 μL, 1.39 mmol was added thereto. The reaction mixture was stirred at 60° C. for 30 minutes. The reaction mixture was cooled to room temperature, H₂O and EtOAc were added, and this mixture was extracted with EtOAc. The organic layer was washed with brine, dried over anhydrous Na₂SO₄, filtered and distilled under reduced pressure to obtain the solid compound, N-(7-chloro-3-(4-methylpiperazin-1-yl)pyrido[3,4-b]pyrazin-2-yl)-2-hydroxyacetamide (220 mg, 94%) in light brown.

LC/MS ESI (+): 337 (M+1)

¹H NMR (400 MHz, DMSO-d₆) δ=8.73; (s, 1H), 7.62; (s, 1H), 7.59; (s, 1H), 7.32; (s, 1H), 4.97; (s, 2H), 3.82-3.75; (m, 4H), 2.49-2.45; (m, 4H), 2.23; (s, 3H)

(b) Synthesis of 8-chloro-4-(4-methylpiperazin-1-yl)imidazo[1,2-a]pyrido[3,4-e]pyrazin-2(1H)-one hydrochloride

N-(7-chloro-3-(4-methylpiperazin-1-yl)pyrido[3,4-b]pyrazin-2-yl)-2-hydroxyacetamide (220 mg, 0.653 mmol) was dissolved in DMF (3.27 mL), and methanesulfonyl chloride (764 μL, 9.80 mmol) and pyridine (1.59 mL, 19.6 mmol) were added thereto at room temperature. The reaction mixture was stirred at 80° C. for 1 hour. The reaction mixture was cooled to room temperature, H₂O and EtOAc were added thereto, and this mixture was extracted with EtOAc. The organic layer was washed with brine, dried over anhydrous Na₂SO₄, filtered and distilled under reduced pressure. The residue was purified by column chromatography (DCM:MeOH=20:1) on amine silica and column chromatography (DCM:MeOH=20:1) on silica, and the fractions containing the product were collected and evaporated to obtain the solid compound, 8-chloro-4-(4-methylpiperazin-1-yl)imidazo[1,2-a]pyrido[3,4-e]pyrazin-2(1H)-one hydrochloride (26.0 mg, 11%) in white.

LC/MS ESI (+): 319 (M+1)

¹H NMR (400 MHz, DMSO-d₆) δ=10.87; (brs, 1H), 8.87; (s, 1H), 7.81; (s, 1H), 5.44; (s, 2H), 4.51; (m, 2H), 3.59-3.38; (m, 4H), 3.33-3.13; (m, 2H), 2.81; (s, 3H)

### Example 29: Synthesis of 2-chloro-6-(4-methylpiperazin-1-yl)imidazo[1,2-a]pyrido[3,2-e]pyrazin-8(9H)-one

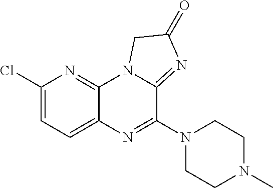 [SMILES: CN1CCN(C2=Nc3ccc(Cl)nc3N3CC(=O)N=C23)CC1]

(a) Synthesis of 6-chloro-1,4-dihydropyrido[2,3-b]pyrazin-2,3-dione

6-Chloropyridin-2,3-diamine (2.00 g, 13.9 mmol) was dissolved in diethyl oxalate (27.9 mL), and this mixture was stirred at 130° C. for 15 hours. The reaction mixture was cooled to room temperature, and the obtained solid was filtered, washed with Et₂O and dried under reduced pressure to obtain the solid compound, 6-chloro-1,4-dihydropyrido[2,3-b]pyrazin-2,3-dione (2.70 g, 96%) in brown.

LC/MS ESI (+): 198 (M+1)

¹H NMR (400 MHz, DMSO-d₆) δ=12.51; (brs, 1H), 12.05; (brs, 1H), 7.46; (d, J=8.2 Hz, 1H), 7.20; (d, J=8.2 Hz, 1H)

(b) Synthesis of 2,3,6-trichloropyrido[2,3-b]pyrazine

POCl₃ (16.9 mL) was added to 6-chloro-1,4-dihydropyrido[2,3-b]pyrazin-2,3-dione (1.00 g, 5.10 mmol), and this mixture was stirred at 130° C. for 24 hours. The reaction mixture was cooled to 0° C. After slow addition of ice water (50.0 mL), the obtained solid was filtered, washed with water and under reduced pressure to obtain the solid compound, 2,3,6-trichloropyrido[2,3-b]pyrazine (945 mg, 80%) in brown.

LC/MS ESI (+): 234 (M+1)

¹H NMR (400 MHz, DMSO-d₆) δ=8.64; (d, J=8.7 Hz, 1H), 8.06; (d, J=8.7 Hz, 1H)

(c) N-(2,6-dichloropyrido[2,3-b]pyrazin-3-yl)-2-hydroxyacetamide

2,3,6-Trichloropyrido[2,3-b]pyrazine (945 mg, 4.00 mmol) and glycolamide (605 mg, 8.10 mmol) were dissolved in DMF (13.4 mL), and DIPEA (1.40 mL, 8.10 mmol) was added thereto at room temperature. The reaction mixture was stirred at 80° C. for 2 hours and distilled under reduced pressure. The residue was purified by column chromatography (H₂O containing 0.1% formic acid:CH₃CN=70:30) on reverse-phase silica, and the fractions containing the product were collected and evaporated to obtain the solid compound, N-(2,6-dichloropyrido[2,3-b]pyrazin-3-yl)-2-hydroxyacetamide (373 mg, 34%) in yellow.

LC/MS ESI (+): 273 (M+1)

¹H NMR (400 MHz, DMSO-d₆) δ=8.49; (d, J=8.5 Hz, 1H), 7.82; (d, J=8.5 Hz, 1H), 7.61; (brs, 1H), 7.37; (brs, 1H), 4.99; (s, 2H)

(d) Synthesis of N-(6-chloro-2-(4-methylpiperazin-1-yl)pyrido[2,3-b]pyrazin-3-yl)-2-hydroxyacetamide

N-(2,6-dichloropyrido[2,3-b]pyrazin-3-yl)-2-hydroxyacetamide (200 mg, 0.700 mmol) was dissolved in DMF (3.70 mL), and 1-methylpiperazine (162 μL, 1.50 mmol) was slowly added thereto at room temperature. The reaction mixture was stirred at 60° C. for 10 minutes and distilled under reduced pressure. The residue was purified by column chromatography (H₂O containing 0.1% formic acid:CH₃CN=70:30) on reverse-phase silica, and the fractions containing the product were collected and evaporated to obtain the solid compound, N-(6-chloro-2-(4-methylpiperazin-1-yl)pyrido[2,3-b]pyrazin-3-yl)-2-hydroxyacetamide (215 mg, 87%) in yellow.

LC/MS ESI (+): 337 (M+1)

¹H NMR (400 MHz, DMSO-d₆) δ=8.05; (d, J=8.5 Hz, 1H), 7.61; (brs, 1H), 7.53; (d, J=8.5 Hz, 1H), 7.29; (brs, 1H), 4.95; (s, 2H), 3.78; (s, 4H), 2.50; (s, 4H), 2.24; (s, 3H)

(e) Synthesis of 2-chloro-6-(4-methylpiperazin-1-yl)imidazo[1,2-a]pyrido[3,2-e]pyrazin-8(9H)-one

N-(6-chloro-2-(4-methylpiperazin-1-yl)pyrido[2,3-b]pyrazin-3-yl)-2-hydroxyacetamide (210 mg, 0.600 mmol) was dissolved in DMF (6.20 mL), and methanesulfonyl chloride (1.00 mL, 12.5 mmol) and TEA (1.70 mL, 12.5 mmol) were added thereto at room temperature. The reaction mixture was stirred at 80° C. for 1 hour and distilled under reduced pressure. The residue was purified by column chromatography (H₂O containing 0.1% formic acid:CH₃CN=70:30) on reverse-phase silica, and the fractions containing the product were collected and evaporated to obtain the solid compound, 2-chloro-6-(4-methylpiperazin-1-yl)imidaz[1,2-a]pyrido[3,2-e]pyrazin-8(9H)-one (142 mg, 71%) in yellow.

LC/MS ESI (+): 319 (M+1)

¹H NMR (400 MHz, DMSO-d₆) δ=8.12; (d, J=8.5 Hz, 1H), 7.62; (d, J=8.5 Hz, 1H), 5.42; (s, 2H), 3.72-3.69; (m, 4H), 2.50-2.46; (m, 4H), 2.22; (s, 3H)

### Example 30: Synthesis of 2-bromo-6-(4-methylpiperazin-1-yl)imidazo[1,2-a]pyrido[3,2-e]pyrazin-8(9H)-one

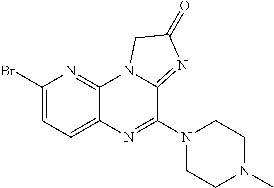 [SMILES: CN1CCN(C2=Nc3ccc(Br)nc3N3CC(=O)N=C23)CC1]

2-Chloro-6-(4-methylpiperazin-1-yl)imidazo[1,2-a]pyrido[3,2-e]pyrazin-8(9H)-one (95.0 mg, 0.300 mmol) was dissolved in CH₃CN (3.00 mL), and bromotrimethylsilane (641 μL, 6.00 mmol) was added thereto at room temperature. The reaction mixture was stirred at 80° C. for 3 days and distilled under reduced pressure. The residue was purified by column chromatography (H₂O containing 0.1% formic acid:CH₃CN=70:30) on reverse-phase silica, and the fractions containing the product were collected and evaporated to obtain the solid compound, 2-bromo-6-(4-methylpiperazin-1-yl)imidazo[1,2-a]pyrido[3,2-e]pyrazin-8(9H)-one (24.0 mg, 22%) in yellow.

LC/MS ESI (+): 363 (M+1)

¹H NMR (400 MHz, DMSO-d₆) δ=8.00; (d, J=8.5 Hz, 1H), 7.72; (d, J=8.5 Hz, 1H), 5.42; (s, 2H), 3.72-3.70; (m, 4H), 2.50-2.46; (m, 4H), 2.22; (s, 3H)

### Example 31: Synthesis of 2-chloro-6-(3-(methylamino)azetidin-1-yl)imidazo[1,2-a]pyrido[3,2-e]pyrazin-8(9H)-one

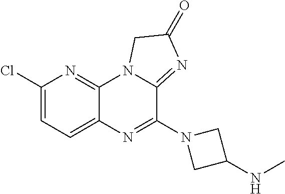 [SMILES: CNC1CN(C2=Nc3ccc(Cl)nc3N3CC(=O)N=C23)C1]

(a) Synthesis of tert-butyl (1-(6-chloro-3-(2-hydroxyacetamido)pyrido[2,3-b]pyrazin-2-yl)azetidin-3-yl)(methyl)carbamate

N-(2,6-dichloropyrido[2,3-b]pyrazin-3-yl)-2-hydroxyacetamide (164 mg, 0.600 mmol) was dissolved in DMF (3.00 mL), and tert-butyl azetidin-3-yl(methyl)carbamate hydrochloride (201 mg, 0.900 mmol) and TEA (335 μL, 2.40 mmol) were slowly added thereto at room temperature. The reaction mixture was stirred at 25° C. for 30 minutes and distilled under reduced pressure. The residue was purified by column chromatography (H₂O containing 0.1% formic acid:CH₃CN=50:50) on reverse-phase silica, and the fractions containing the product were collected and evaporated to obtain the solid compound, tert-butyl (1-(6-chloro-3-(2-hydroxyacetamido)pyrido[2,3-b]pyrazin-2-yl)azetidin-3-yl)(methyl)carbamate (241 mg, 95%) in yellow.

LC/MS ESI (+): 423 (M+1)

¹H NMR (400 MHz, CDCl₃) δ=7.90; (d, J=8.5 Hz, 1H), 7.36; (d, J=8.5 Hz, 1H), 6.31; (brs, 1H), 5.81; (brs, 1H), 5.10; (s, 2H), 4.99; (s, 1H), 4.64-4.60; (m, 2H), 4.46-4.42; (m, 2H), 2.97; (s, 3H), 1.47; (s, 9H)

(b) Synthesis of tert-butyl (1-(2-chloro-8-oxo-8,9-dihydroimidazo[1,2-a]pyrido[3,2-e]pyrazin-6-yl)azetidin-3-yl)(methyl)carbamate

Tert-butyl (1-(6-chloro-3-(2-hydroxyacetamido)pyrido[2,3-b]pyrazin-2-yl)azetidin-3-yl)(methyl)carbamate (600 mg, 1.40 mmol) was dissolved in DMF (14.2 mL), and methanesulfonyl chloride (1.10 mL, 14.2 mmol) and TEA (2.40 mL, 17.0 mmol) was added thereto at room temperature. The reaction mixture was stirred at 80° C. for 2 hours and distilled under reduced pressure. The residue was purified by column chromatography (H₂O containing 0.1% formic acid:CH₃CN=40:60) on reverse-phase silica, and the fractions containing the product were collected and evaporated to obtain the solid compound, tert-butyl (1-(2-chloro-8-oxo-8,9-dihydroimidazo[1,2-a]pyrido[3,2-e]pyrazin-6-yl)azetidin-3-yl)(methyl)carbamate (138 mg, 24%) in brown.

LC/MS ESI (+): 405 (M+1)

¹H NMR (400 MHz, CDCl₃) δ=7.94; (d, J=8.5 Hz, 1H), 7.42; (d, J=8.5 Hz, 1H), 5.23; (s, 2H), 5.08; (s, 1H), 4.64-4.58; (m, 2H), 4.44-4.40; (m, 2H), 2.98; (s, 3H), 1.48; (s, 9H)

(c) Synthesis of 2-chloro-6-(3-(methylamino)azetidin-1-yl)imidazo[1,2-a]pyrido[3,2-e]pyrazin-8(9H)-one

Tert-butyl (1-(2-chloro-8-oxo-8,9-dihydroimidazo[1,2-a]pyrido[3,2-e]pyrazin-6-yl)azetidin-3-yl)(methyl)carbamate (100 mg, 0.300 mmol) was dissolved in DCM (1.20 mL), and TFA (567 μL, 7.40 mmol) was added thereto at 0° C. The reaction mixture was stirred at room temperature for 10 minutes and distilled under reduced pressure. The residue was purified by column chromatography (H₂O containing 0.1% formic acid:CH₃CN=70:30) on reverse-phase silica, and the fractions containing the product were collected and evaporated to obtain the solid compound, 2-chloro-6-(3-(methylamino)azetidin-1-yl)imidazo[1,2-a]pyrido[3,2-e]pyrazin-8(9H)-one (65.0 mg, 86%) in white.

LC/MS ESI (+): 305 (M+1)

¹H NMR (400 MHz, DMSO-d₆) δ=8.01; (d, J=8.5 Hz, 1H), 7.54; (d, J=8.5 Hz, 1H), 5.38; (s, 2H), 4.56-4.33; (m, 2H), 4.11-3.86; (m, 2H), 4.61-3.55; (m, 1H), 2.36; (brs, 1H), 2.25; (s, 3H)

### Example 32: Synthesis of 2-bromo-6-(3-(methylamino)azetidin-1-yl)imidazo[1,2-a]pyrido[3,2-e]pyrazin-8(9H)-one

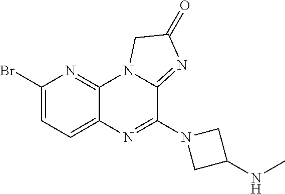 [SMILES: CNC1CN(C2=Nc3ccc(Br)nc3N3CC(=O)N=C23)C1]

(a) Synthesis of 6-bromo-1,4-dihydropyrido[2,3-b]pyrazin-2,3-dione

6-Bromopyridin-2,3-diamine (1.00 g, 5.20 mmol) was dissolved in diethyl oxalate (10.4 mL), and this mixture was stirred at 130° C. for 15 hours. The reaction mixture was cooled to room temperature, and the obtained solid was filtered, washed with Et₂O and dried under reduced pressure to obtain the solid compound, 6-bromo-1,4-dihydropyrido[2,3-b]pyrazin-2,3-dione (1.20 g, 98%) in brown.

LC/MS ESI (+): 242 (M+1)

¹H NMR (400 MHz, DMSO-d₆) δ=12.51; (brs, 1H), 12.05; (brs, 1H), 7.37; (d, J=8.2 Hz, 1H), 7.32; (d, J=8.2 Hz, 1H)

(b) Synthesis of 2,3,6-tribromopyrido[2,3-b]pyrazine

6-Bromo-1,4-dihydropyrido[2,3-b]pyrazin-2,3-dione (1.10 g, 4.50 mmol) and DMF (18.0 μL, 0.200 mmol) were dissolved in DCE (11.4 mL), and POBr₃ (3.90 g, 13.6 mmol) was added thereto at room temperature. The reaction mixture was stirred at 100° C. for 15 hours and distilled under reduced pressure. The residue was purified by column chromatography (H₂O containing 0.1% formic acid:CH₃CN=40:60) on reverse-phase silica, and the fractions containing the product were collected and evaporated to obtain the solid compound, 2,3,6-tribromopyrido[2,3-b]pyrazine (795 mg, 48%) in yellow.

LC/MS ESI (+): 366 (M+1)

¹H NMR (400 MHz, DMSO-d₆) δ=8.51; (d, J=8.7 Hz, 1H), 8.16; (d, J=8.7 Hz, 1H)

(c) Synthesis of N-(2,6-dibromopyrido[2,3-b]pyrazin-3-yl)-2-hydroxyacetamide

2,3,6-Tribromopyrido[2,3-b]pyrazine (838 mg, 2.30 mmol) and glycolamide (342 mg, 4.60 mmol) were dissolved in DMF (9.10 mL), and DIPEA (0.800 mL, 4.60 mmol) was added thereto at room temperature. The reaction mixture was stirred at 80° C. for 2 hours and distilled under reduced pressure. The residue was purified by column chromatography (H₂O containing 0.1% formic acid:CH₃CN=70:30) on reverse-phase silica, and the fractions containing the product were collected and evaporated to obtain the solid compound, N-(2,6-dibromopyrido[2,3-b]pyrazin-3-yl)-2-hydroxyacetamide (230 mg, 28%) in yellow.

LC/MS ESI (+): 361 (M+1)

¹H NMR (400 MHz, DMSO-d₆) δ=8.39; (d, J=8.5 Hz, 1H), 7.92; (d, J=8.5 Hz, 1H), 7.60; (brs, 1H), 7.37; (brs, 1H), 4.98; (s, 2H)

(d) Synthesis of tert-butyl (1-(6-bromo-3-(2-hydroxyacetamido)pyrido[2,3-b]pyrazin-2-yl)azetidin-3-yl)(methyl)carbamate

N-(2,6-dibromopyrido[2,3-b]pyrazin-3-yl)-2-hydroxyacetamide (225 mg, 0.600 mmol) was dissolved in DMF (3.10 mL), and tert-butyl azetidin-3-yl(methyl)carbamate hydrochloride (208 mg, 0.900 mmol) and TEA (347 μL, 2.50 mmol) were slowly added thereto at room temperature. The reaction mixture was stirred at 25° C. for 10 minutes and distilled under reduced pressure. The residue was purified by column chromatography (H₂O containing 0.1% formic acid:CH₃CN=50:50) on reverse-phase silica, and the fractions containing the product were collected and evaporated to obtain the solid compound, tert-butyl (1-(6-bromo-3-(2-hydroxyacetamido)pyrido[2,3-b]pyrazin-2-yl)azetidin-3-yl)(methyl) carbamate (255 mg, 88%) in yellow.

LC/MS ESI (+): 467 (M+1)

¹H NMR (400 MHz, DMSO-d₆) δ=7.87; (d, J=8.4 Hz, 1H), 7.59; (d, J=8.4 Hz, 1H), 7.56; (brs, 1H), 7.34; (brs, 1H), 5.03-4.76; (m, 1H), 4.87; (s, 2H), 4.61-4.28; (m, 4H), 2.89; (s, 3H), 1.40; (s, 9H)

(e) Synthesis of tert-butyl (1-(2-bromo-8-oxo-8,9-dihydroimidazo[1,2-a]pyrido[3,2-e]pyrazin-6-yl)azetidin-3-yl)(methyl)carbamate

Tert-butyl (1-(6-bromo-3-(2-hydroxyacetamido)pyrido[2,3-b]pyrazin-2-yl)azetidin-3-yl)(methyl)carbamate (250 mg, 0.500 mmol) was dissolved in DMF (5.40 mL), and methanesulfonyl chloride (0.400 mL, 5.40 mmol) and TEA (0.900 mL, 6.40 mmol) were added thereto at room temperature. The reaction mixture was stirred at 80° C. for 1 hour and distilled under reduced pressure. The residue was purified by column chromatography (H₂O containing 0.1% formic acid:CH₃CN=40:60) on reverse-phase silica, and the fractions containing the product were collected and evaporated to obtain the solid compound, tert-butyl (1-(2-bromo-8-oxo-8,9-dihydroimidazo[1,2-a]pyrido[3,2-e]pyrazin-6-yl)azetidin-3-yl)(methyl)carbamate (67.0 mg, 28%) in yellow.

LC/MS ESI (+): 449 (M+1)

¹H NMR (400 MHz, DMSO-d₆) δ=7.93; (d, J=8.4 Hz, 1H), 7.66; (d, J=8.4 Hz, 1H), 5.37; (s, 2H), 5.01-4.69; (m, 1H), 4.59-4.26; (m, 4H), 2.88; (s, 3H), 1.40; (s, 9H)

(f) Synthesis of 2-bromo-6-(3-(methylamino)azetidin-1-yl)imidazo[1,2-a]pyrido[3,2-e]pyrazin-8(9H)-one

Tert-butyl (1-(2-bromo-8-oxo-8,9-dihydroimidazo[1,2-a]pyrido[3,2-e]pyrazin-6-yl)azetidin-3-yl)(methyl)carbamate (60.0 mg, 0.130 mmol) was dissolved in DCM (0.700 mL), and TFA (307 μL, 4.00 mmol) was added thereto at 0° C. The reaction mixture was stirred at room temperature for 1 hour and distilled under reduced pressure. The residue was purified by column chromatography (H₂O containing 0.1% formic acid:CH₃CN=70:30) on reverse-phase silica, and the fractions containing the product were collected and evaporated to obtain the solid compound, 2-bromo-6-(3-(methylamino)azetidin-1-yl)imidazo[1,2-a]pyrido[3,2-e]pyrazin-8(9H)-one (43.0 mg, 92%) in white.

LC/MS ESI (+): 349 (M+1)

¹H NMR (400 MHz, DMSO-d₆) δ=7.90; (d, J=8.4 Hz, 1H), 7.64; (d, J=8.4 Hz, 1H), 5.38; (s, 2H), 4.56-4.33; (m, 2H), 4.11-3.86; (m, 2H), 4.61-3.55; (m, 1H), 2.41; (brs, 1H), 2.25; (s, 3H)

### Experimental Example 1: Analysis for Binding Affinity of Human Histamine 4 Receptor (hH4R)

The compounds prepared in the above Examples, the compound of Example 55 of International Publication No. WO 2010/030785 and the compound of Example 4 of International Publication No. WO 2013/048214 were diluted by 1,000 fold (v/w) with DMSO, and then 1 mL of the diluted compound solution was mixed with 99 mL of the analysis buffer solution (50 mM tris-HCl pH 7.4, 5 mM EDTA) to obtain concentration of 1 μM. 20 mL of the prepared compound solution was transferred to each well of a 96-well plate, and then 20 mL of 100 μM histamine diluted with analysis buffer solution and 1% DMSO were transferred to each well to calculate non-specific binding and total binding degree. 15 μg of human histamine 4 receptor-overexpressed cell membrane (Multispan) was diluted into 160 mL of analysis buffer solution and that was transferred to each well. [³H] labeled histamine (PerkinElmer) was diluted into 10 nM concentration, 20 mL was allocated in each well, and then it was kept in a 27° C. incubator for 30 minutes. After the reaction, 100 mL of the mixture was transferred to a glass fiber plate in which 0.5% polyethylene amine was pre-soaked, and then non-binding [³H] labeled histamine was removed in vacuum. After 6 times washing with 200 mL of washing buffer solution (50 mM tris-HCl, pH 7.4), the plate was dried in a 37° C. oven for 18 hours. 100 mL of betaScint cocktail solution was added to each well, and after 10 minutes CPM (count per minute) value of [³H] labeled histamine was measured by the use of Micro beta2™. The binding affinity (% inhibition) of the human histamine 4 receptor for the compounds of the present invention was analyzed by an Excel program, and the results of analysis are represented in Table 1.

TABLE 1

| | Human histamine | | Human histamine |
|---|---|---|---|
| | 4 receptor (hH4R) | | 4 receptor (hH4R) |
| | binding affinity | | binding affinity |
| | (% inhibition | | (% inhibition |
| Example | at 1 μM) | Example | at 1 μM) |
| 1 | 79.7 | 17 | 87.1 |
| 2 | 39.5 | 18 | 34.1 |
| 3 | 9.80 | 19 | 96.0 |
| 4 | −4.38 | 20 | 20.5 |
| 5 | 55.9 | 21 | 95.9 |
| 6 | 42.6 | 22 | 86.3 |
| 7 | −1.12 | 23 | 89.4 |
| 8 | 88.9 | 24 | 91.9 |
| 9 | 94.4 | 25 | 96.9 |
| 10 | 98.1 | 26 | 93.4 |
| 11 | 44.8 | 27 | 22.6 |
| 12 | 101 | 28 | 96.8 |
| 13 | 95.8 | 29 | 97.3 |
| 14 | 52.6 | 30 | 93.2 |
| 15 | 99.2 | 31 | 97.7 |
| 16 | −5.98 | 32 | 97.4 |
| Example 55 | 92.4 | Example 4 | 86.3 |
| WO 2010/ | | WO 2013/ | |
| 030785 | | 048214 | |

### Experimental Example 2: Test for Pharmacokinetics

### Preparation of Drug

As the administration solution for mice of the compounds prepared in the above

Examples, the compound of Example 55 of International Publication No. WO 2010/030785 and the compound of Example 4 of International Publication No. WO 2013/048214, 20% hydroxypropyl-β-cyclodextrin solution (99.75%) and 2N HCl (0.25%) were added sequentially to make 5 mg/mL. Provided that in the case of the compound of Example 10, 99.5% of 20% hydroxypropyl-β-cyclodextrin solution and 0.5% of 2N HCl (0.25%) were added.

### Experiment

The weight of each ICR mouse was properly 20-30 g. Dosage of the compound of the present invention was 10 mL/kg, and administered orally using Zonde. Blood collection was performed at 0.5, 1, 2, 4, 7 and 24 hours by orbital venous blood collection using a capillary tube coated with an anticoagulant, and then plasma was isolated using a centrifuge and kept in a freezer.

### Analysis

Plasma collected from animals and standard concentration material were pre-treated using solid-phase extraction, and concentration of the compound of the invention was determined using a liquid chromatography mass spectrometer (Agilent HPLC, API-4000 Qtrap). According to the resulting concentration value, the pharmacokinetics parameter was found using WinNonlin (Version 7.0) and half-life (t_{1/2}), maximum blood concentration (Cₘₐₓ) and area under the curve (AUCₐₗₗ) are represented in Table 2.

TABLE 2

| | Mouse pharmacokinetics | | |
|---|---|---|---|
| | t_{1/2} | Cₘₐₓ | AUCₐₗₗ |
| Example | (h) | (μg/mL) | (μg · hr/mL) |
| 1 | 3.81 | 2.28 | 8.65 |
| 10 | 2.04 | 0.360 | 0.920 |
| 12 | 1.73 | 0.570 | 1.39 |
| 13 | NC* | 2.50 | 30.5 |
| Example 55 | 3.07 | 0.676 | 1.68 |
| WO 2010/030785 | | | |
| Example 4 | 7.61 | 4.63 | 23.6 |
| WO 2013/048214 | | | |

*NC: not calculated

### Experimental Example 3: Histamine-Induced Itching Model in ICR Mice

### Animal

Female, ICR mice (8 weeks old) were purchased from OrientBio Co., Ltd. The animals were housed under conditions of controlled temperature (23±3° C.), humidity (50±5%) and lighting with food and water available ad libitum. Water and food were stopped 1 hour before the experiment.

### Experiment: Itch-Inducement and Measurement

For easy intradermal injection of pruritogen (histamine, dissolved in saline injection solution to make the concentration of 300 nmol per 40 μL) the hair was clipped using clippers (8000AD, THRIVE) over the rostral part of the back 24 hours before the experiment under inhalation anesthesia with isoflurane. Mice (n=10 per group) were divided into 5 groups (normal group, control group and 3 experimental groups). The animals were randomized for similar body weight distribution. 30 minutes before the administration of histamine, a vehicle (20% cyclodextrin in second distilled water) was administered orally to the normal group and the control group, and the compound prepared in Example 13, the compound of Example 55 of International Publication No. WO 2010/030785 and the compound of Example 4 of International Publication No. WO 2013/048214 were administered orally to the experimental groups (dissolved in excipients at the dose of 50 mg/kg). Because blood concentration is maintained for 24 hours, histamine was administered 7 hours after oral administration. Immediately after histamine administration, the animals were place in observation cages with an independent space between individuals, and then videoed for 20 minutes using a camera (PowerShot N2, Canon). At the end of the filming, the number of scratches of the test animals was counted during 20 minutes after histamine administration using the recorded video. The number of scratches was counted a series of actions from scratching with the rear foot of the animal to the time of taking the rear foot to the mouth as one time (J. Allergy Clin. Immunol. 2007, 19(1), 176-183). All data were analyzed by using Excel and Prism, and the number of scratching of each group is expressed as the mean±S.E.M. The inhibitory effect of compounds is shown as a percentage of the maximal response to histamine (100% control group). Statistical analysis was analyzed using one-way ANOVA with Dunnett test. Values of p<0.05 were considered statistically significant. The inhibitory effects of the compound are represented in Table 3.

$$\begin{matrix}Inhibitory effect of \\ the compound ( \% )\end{matrix} = \frac{\begin{matrix}Number of scratching \\ of control group\end{matrix} - \begin{matrix}Number of scratching \\ of experimental group\end{matrix}}{\begin{matrix}Number of scratching \\ of control group\end{matrix}} \times 100$$

TABLE 3

Inhibitory rate (%)

| Example | After 30 minutes | After 7 hours |
|---|---|---|
| 13 | 131 | 99.0 |
| Example 55 | 89.3 | 66.7 |
| WO 2010/030785 | | |
| Example 4 | 110 | 73.7 |
| WO 2013/048214 | | |

1. A heterocyclic compound of the following Formula 1, or a pharmaceutically acceptable salt or isomer thereof: 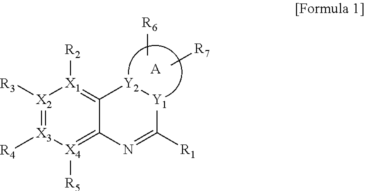
wherein
each of X₁, X₂, X₃ and X₄ is independently C or N;
R₁ is a saturated or unsaturated 3-12-membered mono- or poly-heterocyclyl containing 1-3 heteroatoms (preferably the heteroatoms selected from N, O and S), wherein R₁ is unsubstituted or substituted with 1-3 substituents selected from —C₁-C₆ alkyl and -amino-C₁-C₆ alkyl;
R2, R3, R4 and R5 may be the same or different; and each of them is independently selected from —H, —C₁-C₆ alkyl, —C₁-C₆ haloalkyl, —C₁-C₆ perhaloalkyl, -amino-C₁-C₆ alkyl, —C₃-C₈cycloalkyl, -halogen (—F, —Cl, —Br, —I), —CN, —C₁-C₆ alkoxy, —C₁-C₆ haloalkoxy, —C₁-C₆ perhaloalkoxy, —C₂-C₇ alkenyl, —C₂-C₈ alkynyl, -amino, -aceto, -amido, -sulfonamide, -sulfonyl, -aminosulfonyl-C₁-C₆ alkyl, —C₁-C₆ alkylcarboxyl, -carboxyl (—COOH), —C₁-C₆ acyl, —OH, -nitro (—NO₂), —C₆-C₁₀ aryl, -heterocyclyl, and —O—C₁-C₆ alkyl-heterocyclyl, wherein the heterocyclyl is a saturated or unsaturated 3-6-membered heterocyclyl containing 1-3 heteroatoms (preferably heteroatoms selected from N, O and S);
provided that when X₁ is N, R₂ does not exist; when X₂ is N, R₃ does not exist;
when X₃ is N, R₄ does not exist; and when X₄ is N, R₅ does not exist; and when all of X₁, X₂, X₃ and X₄ are C, R₃ is not hydrogen or fluorine (F);
each of Y₁ and Y₂ is independently C or N;
A ring is a saturated or unsaturated 5- or 6-membered heterocycle containing at least 2 heteroatoms (preferably the heteroatoms selected from N, O and S); and
each of R₆ and R₇ is independently oxo (═O) or ═NH, and one of R₆ and R₇ may not exist;
wherein each of the alkyl, cycloalkyl, heterocyclyl, alkoxy, alkenyl, alkynyl, acyl and aryl groups may be independently unsubstituted or substituted with one or more substituents (for example, 1-3 substituents) selected from the group consisting of —C₁-C₄ alkyl, -halogen (—F, —Cl, —Br, —I), —CN, —C₁-C₄alkoxy, -amino, -amido, -carboxyl (—COOH), —C₁-C₆ acyl, —OH, -nitro (—NO₂), heterocyclyl and phenyl, wherein the heterocyclyl is a saturated or unsaturated 3-6-membered heterocyclyl containing 1-3 heteroatoms (preferably, the heteroatoms selected from N, O and S).

2. The heterocyclic compound, or a pharmaceutically acceptable salt or isomer thereof according to claim 1, wherein
each of X₁, X₂, X₃ and X₄ is independently C or N;
R₁ is a saturated or unsaturated 3-10-membered mono- or poly-heterocyclyl containing 1-3 heteroatoms selected from N, O and S, wherein the heterocyclyl is unsubstituted or substituted with 1 or 2 substituents selected from —C₁-C₆ alkyl and -amino-C₁-C₆ alkyl;
R₂, R₃, R₄ and R₅ may be the same or different; and each of them is independently selected from —H, —C₁-C₆ alkyl, —C₁-C₆ haloalkyl, —C₁-C₆ perhaloalkyl, -amino-C₁-C₆ alkyl, —C₃-C₈ cycloalkyl, -halogen, —CN, —C₁-C₆ alkoxy, —C₁-C₆ haloalkoxy, —C₁-C₆ perhaloalkoxy, -amino, -aceto, -sulfonamino, -sulfonyl, -aminosulfonyl-C₁-C₆ alkyl, —C₁-C₆ alkylcarboxyl, -carboxyl, —OH, -nitro, —C₆-C₁₀ aryl, -heterocyclyl, and —O—C₁-C₆ alkyl-heterocyclyl, wherein the heterocyclyl is a saturated or unsaturated 3-6-membered heterocyclyl containing 1-3 heteroatoms selected from N, O and S;
each of Y₁ and Y₂ is independently C or N;
A ring is a saturated or unsaturated 5- or 6-membered heterocycle containing 2-4 heteroatoms selected from N, O and S; and
each of R₆ and R₇ is independently oxo or ═NH, and one of R₆ and R₇ may not exist.

3. The heterocyclic compound, or a pharmaceutically acceptable salt or isomer thereof according to claim 1, wherein X₁ and X₂ are C, and each of X₃ and X₄ is independently C or N.

4. The heterocyclic compound, or a pharmaceutically acceptable salt or isomer thereof according to claim 1, wherein each of X₁ and X₂ is independently C or N, and X₃ and X₄ are C.

5. The heterocyclic compound, or a pharmaceutically acceptable salt or isomer thereof according to claim 1, wherein R₁ is a saturated or unsaturated 3-10-membered mono- or poly-heterocyclyl containing 1-3 heteroatoms selected from N and O, wherein the heterocyclyl is unsubstituted or substituted with 1 or 2 substituents selected from —C₁-C₄ alkyl and -amino-C₁-C₄ alkyl.

6. The heterocyclic compound, or a pharmaceutically acceptable salt or isomer thereof according to claim 1, wherein R₂, R₃, R₄ and R₅ may be the same or different; and each of them is independently selected from —H, —C₁-C₆ alkyl, —C₁-C₆ haloalkyl, —C₁-C₆ perhaloalkyl, -amino-C₁-C₆ alkyl, -halogen, —CN, —C₁-C₆ alkoxy, —C₁-C₆ haloalkoxy, —C₁-C₆ perhaloalkoxy, -amino, -aceto, -sulfonamino, -sulfonyl, -aminosulfonyl-C₁-C₆ alkyl, —C₁-C₆ alkylcarboxyl, -carboxyl, —OH, -nitro, and -heterocyclyl, wherein the heterocyclyl is a saturated or unsaturated 5 or 6-membered heterocyclyl containing 1-3 heteroatoms selected from N, O and S.

7. The heterocyclic compound, or a pharmaceutically acceptable salt or

1. thereof according to claim 1, wherein A ring is a saturated or unsaturated 5- or 6-membered heterocycle containing 2 or 3 heteroatoms selected from N and S.

8. The heterocyclic compound, or a pharmaceutically acceptable salt or isomer thereof according to claim 1, wherein R6 is oxo or ═NH, and R7 does not exist.

9. The heterocyclic compound, or a pharmaceutically acceptable salt or isomer thereof according to claim 1, wherein R6 and R7 are oxo.

10. The heterocyclic compound, or a pharmaceutically acceptable salt or isomer thereof according to claim 1, wherein the heterocyclic compound is selected from the group consisting of:
8-bromo-4-(4-methylpiperazin-1-yl)imidazo[1,2-a]pyrido[2,3-e]pyrazin-2(1H)-one;
8-bromo-4-(3-(methylamino)azetidin-1-yl)imidazo[1,2-a]pyrido[2,3-e]pyrazin-2(1H)-one;
(R)-8-bromo-4-(3-(methylamino)pyrrolidin-1-yl)imidazo[1,2-a]pyrido[2,3-e]pyrazin-2(1H)-one;
(S)-8-bromo-4-(3-(methylamino)pyrrolidin-1-yl)imidazo[1,2-a]pyrido[2,3-e]pyrazin-2(1H)-one;
8-bromo-4-(hexahydropyrrolo[1,2-a]pyrazin-2(1H)-yl)imidazo[1,2-a]pyrido[2,3-e]pyrazin-2(1H)-one;
8-bromo-4-(4-methylpiperazin-1-yl)pyrido[2,3-e][1,2,4]triazolo[4,3-a]pyrazin-1(2H)-one;
8-bromo-4-(4-methylpiperazin-1-yl)pyrido[2,3-e][1,2,4]triazolo[1,5-a]pyrazin-2(1H)-one;
8-bromo-7-chloro-4-(4-methylpiperazin-1-yl)imidazo[1,2-a]pyrido[2,3-e]pyrazin-2(1H)-one;
8-bromo-7-fluoro-4-(4-methylpiperazin-1-yl)imidazo[1,2-a]pyrido[2,3-e]pyrazin-2(1H)-one;
4-(4-methylpiperazin-1-yl)-8-nitroimidazo[1,2-a]quinoxalin-2(1H)-one;
8-amino-4-(4-methylpiperazin-1-yl)imidazo[1,2-a]quinoxalin-2(1H)-one;
8-bromo-4-(4-methylpiperazin-1-yl)imidazo[1,2-a]quinoxalin-2(1H)-one;
8-bromo-4-(3-(methylamino)azetidin-1-yl)imidazo[1,2-a]quinoxalin-2(1H)-one;
9-bromo-5-(4-methylpiperazin-1-yl)-1H-[1,2,4]triazino[4,3-a]quinoxalin-2(3H)-one;
8,9-dibromo-4-(4-methylpiperazin-1-yl)imidazo[1,2-a]quinoxalin-2(1H)-one;
N-(4-(3-(methylamino)azetidin-1-yl)-2-oxo-1,2-dihydroimidazo[1,2-a]quinoxalin-8-yl)methanesulfonamide;
8-chloro-4-(4-methylpiperazin-1-yl)imidazo[1,2-a]pyrido[2,3-e]pyrazin-2(1H)-one;
8-amino-4-(3-(methylamino)azetidin-1-yl)imidazo[1,2-a]quinoxalin-2(1H)-one;
8-chloro-4-(3-(methylamino)azetidin-1-yl)imidazo[1,2-a]quinoxalin-2(1H)-one;
8-chloro-4-(3-(methylamino)azetidin-1-yl)imidazo[1,2-a]pyrido[2,3-e]pyrazin-2(1H)-one;
8-bromo-4-(3-(methylamino)azetidin-1-yl)imidazo[1,2-a]quinoxalin-2(1H)-one hydrochloride;
3-chloro-6-(4-methylpiperazin-1-yl)imidazo[1 ,2-a]pyrido[4,3-e]pyrazin-8(9H)-one;
8-chloro-4-(3-(methylamino)azetidin-1-yl)imidazo[1,2-a]pyrido[3,4-e]pyrazin-2(1H)-one hydrochloride;
8-chloro-4-(3-(methylamino)azetidin-1-yl)imidazo[1,2-a]pyrido[3,4-e]pyrazin-2(1H)-one;
8-bromo-4-(4-methylpiperazin-1-yl)imidazo[1,2-a]pyrido[3,4-e]pyrazin-2(1H)-one;
8-bromo-4-(3-(methylamino)azetidin-1-yl)imidazo[1,2-a]pyrido[3,4-e]pyrazin-2(1H)-one;
8-bromo-4-(4-methylpiperazin-1-yl)-1H-pyrido[2,3-e][1,2,4]thiadiazolo[4,3-a]pyrazine 2,2-dioxide;
8-chloro-4-(4-methylpiperazin-1-yl)imidazo[1,2-a]pyrido[3,4-e]pyrazin-2(1H)-one hydrochloride;
2-chloro-6-(4-methylpiperazin-1-yl)imidazo[1,2-a]pyrido[3,2-e]pyrazin-8(9H)-one;
2-bromo-6-(4-methylpiperazin-1-yl)imidazo[1,2-a]pyrido[3,2-e]pyrazin-8(9H)-one;
2-chloro-6-(3-(methylamino)azetidin-1-yl)imidazo[1,2-a]pyrido[3,2-e]pyrazin-8(9H)-one; and
2-bromo-6-(3-(methylamino)azetidin-1-yl)imidazo[1,2-a]pyrido[3,2-e]pyrazin-8(9H)-one.

11. A pharmaceutical composition comprising a heterocyclic compound, or a pharmaceutically acceptable salt or isomer thereof as defined in claim 1 as an active ingredient, and a pharmaceutically acceptable carrier.

12. The pharmaceutical composition according to claim 11, wherein the composition exhibits a human histamine 4 receptor (hH4R) inhibition activity.

13. The pharmaceutical composition according to claim 11, wherein the composition is for the prevention or treatment of a disease selected from the group consisting of inflammatory diseases, autoimmune diseases, allergic diseases, ocular diseases, skin diseases, respiratory diseases, pain diseases, cardiac diseases, and human histamine 4 receptor (hH4R)-related diseases.

14. The pharmaceutical composition according to claim 13, wherein the composition is for the prevention or treatment of a disease selected from the group consisting of inflammatory disorder, allergy, pain, nasal polyps, rhinitis, chronic sinusitis, nasal congestion, nasal itch, asthma, chronic obstructive pulmonary disease, rheumatoid arthritis, atopic dermatitis, psoriasis, eczema, pruritus, itchy skin, urticaria, idiopathic chronic urticaria, scleroderma, conjunctivitis, keratoconjunctivitis, ocular inflammation, dry eye, cardiac dysfunction, age-related macular degeneration, arrhythmia, atherosclerosis, multiple sclerosis, inflammatory bowel disease (colitis, Crohn's disease, ulcerative colitis), inflammatory pain, neuropathic pain, osteoarthritic pain, autoimmune thyroid disease, immune-mediated diabetes, lupus, post-operative adhesions, vestibular disorders and cancer.
